SEQUENCE LISTING (585 sequences)

SEQ ID NO: 1 - protein, 15 aa, synthetic construct
  REGION 1..15: note = Description of Artificial Sequence: Synthetic peptide
GYSTPPKKKRKVEDP

SEQ ID NO: 2 - protein, 12 aa, synthetic construct
  REGION 1..12: note = Description of Artificial Sequence: Synthetic peptide
GYSTPPKTRRRP

SEQ ID NO: 3 - protein, 10 aa, synthetic construct
  REGION 1..10: note = Description of Artificial Sequence: Synthetic peptide
GYSTPGRKKR

SEQ ID NO: 4 - protein, 13 aa, synthetic construct
  REGION 1..13: note = Description of Artificial Sequence: Synthetic peptide
GYSTPRRNRRRRW

SEQ ID NO: 5 - protein, 15 aa, synthetic construct
  REGION 1..15: note = Description of Artificial Sequence: Synthetic peptide
PDEVKRKKKPPTSYG

SEQ ID NO: 6 - protein, 12 aa, synthetic construct
  REGION 1..12: note = Description of Artificial Sequence: Synthetic peptide
PRRRTKPPTSYG

SEQ ID NO: 7 - protein, 10 aa, synthetic construct
  REGION 1..10: note = Description of Artificial Sequence: Synthetic peptide
RKKRGPTSYG

SEQ ID NO: 8 - protein, 13 aa, synthetic construct
  REGION 1..13: note = Description of Artificial Sequence: Synthetic peptide
WRRRRNRRPTSYG

SEQ ID NO: 9 - protein, 17 aa, synthetic construct
  REGION 1..17: note = Description of Artificial Sequence: Synthetic peptide
GYGPPKKKRKVEAPYKA

SEQ ID NO: 10 - protein, 9 aa, synthetic construct
  REGION 1..9: note = Description of Artificial Sequence: Synthetic peptide
PAAKRVKLD

SEQ ID NO: 11 - protein, 11 aa, synthetic construct
  REGION 1..11: note = Description of Artificial Sequence: Synthetic peptide
RQRRNELKRSP

SEQ ID NO: 12 - protein, 16 aa, synthetic construct
  REGION 1..16: note = Description of Artificial Sequence: Synthetic peptide
KRPAATKKAGQAKKKK

SEQ ID NO: 13 - protein, 24 aa, synthetic construct
  REGION 1..24: note = Description of Artificial Sequence: Synthetic peptide
VRKKRKTEEESPLKDKDAKKSKQE

SEQ ID NO: 14 - protein, 24 aa, synthetic construct
  REGION 1..24: note = Description of Artificial Sequence: Synthetic peptide
RLRRDAGGRGGVYEHLGGAPRRRK

SEQ ID NO: 15 - protein, 19 aa, synthetic construct
  REGION 1..19: note = Description of Artificial Sequence: Synthetic peptide
KRKGDEVDGVDECAKKSKK

SEQ ID NO: 16 - protein, 38 aa, synthetic construct
  REGION 1..38: note = Description of Artificial Sequence: Synthetic polypeptide
NQSSNFGPMKGGNFGGRSSGPYGGGGQYFAKPRNQGGY

SEQ ID NO: 17 - protein, 27 aa, synthetic construct
  REGION 1..27: note = Description of Artificial Sequence: Synthetic peptide
GGKRTADGSEFESPKKARKVEAYPKAW

SEQ ID NO: 18 - protein, 27 aa, synthetic construct
  REGION 1..27: note = Description of Artificial Sequence: Synthetic peptide
GGKRTADGSEFESPKKKRAVEAYPKAW

SEQ ID NO: 19 - protein, 27 aa, synthetic construct
  REGION 1..27: note = Description of Artificial Sequence: Synthetic peptide
GGKRTADGSEFESPKKKAKVEAYPKAW

SEQ ID NO: 20 - protein, 28 aa, synthetic construct
  REGION 1..28: note = Description of Artificial Sequence: Synthetic peptide
GGKRTADGSEFESPKKKRKVEAPYKAWK

SEQ ID NO: 21 - protein, 26 aa, synthetic construct
  REGION 1..26: note = Description of Artificial Sequence: Synthetic peptide
GGKRTADGSEFESPKKKRKVEYKAWK

SEQ ID NO: 22 - protein, 20 aa, synthetic construct
  REGION 1..20: note = Description of Artificial Sequence: Synthetic peptide
GYGPAAKRVKLDEAYPKAWK

SEQ ID NO: 23 - protein, 29 aa, synthetic construct
  REGION 1..29: note = Description of Artificial Sequence: Synthetic peptide
GGKRTADGSEFEPAAKRVKLDEAYPKAWK

SEQ ID NO: 24 - protein, 24 aa, synthetic construct
  REGION 1..24: note = Description of Artificial Sequence: Synthetic peptide
GTGPKKKRKVGGGGYGPKKKRLVG

SEQ ID NO: 25 - protein, 25 aa, synthetic construct
  REGION 1..25: note = Description of Artificial Sequence: Synthetic peptide
KRPAATKKAGQAKKKKLEAYPKAWK

SEQ ID NO: 26 - protein, 16 aa, synthetic construct
  REGION 1..16: note = Description of Artificial Sequence: Synthetic peptide
ATKGTKRSYEQMETGE

SEQ ID NO: 27 - protein, 12 aa, synthetic construct
  REGION 1..12: note = Description of Artificial Sequence: Synthetic peptide
GKWERKPIRCAS

SEQ ID NO: 28 - protein, 29 aa, synthetic construct
  REGION 1..29: note = Description of Artificial Sequence: Synthetic peptide
GYGKRTADSQHSTPPKKKRKVEAPYKAWK

SEQ ID NO: 29 - protein, 26 aa, synthetic construct
  REGION 1..26: note = Description of Artificial Sequence: Synthetic peptide
KRTADSQHSTPPKKKRKVEAPYKAWK

SEQ ID NO: 30 - protein, 46 aa, synthetic construct
  REGION 1..46: note = Description of Artificial Sequence: Synthetic polypeptide
GYGPPKKKRKVEAPYKAWKWAKYPAMRRAHHRRRRASHRRRTTTGT

SEQ ID NO: 31 - protein, 43 aa, synthetic construct
  REGION 1..43: note = Description of Artificial Sequence: Synthetic polypeptide
GYGPPKKKRKVEAPYKAWKRGARRYSKMKRRRRRVARRHRRRP

SEQ ID NO: 32 - protein, 23 aa, synthetic construct
  REGION 1..23: note = Description of Artificial Sequence: Synthetic peptide
FWGYGYGPPKKKRKVEAPYKAWK

SEQ ID NO: 33 - protein, 28 aa, synthetic construct
  REGION 1..28: note = Description of Artificial Sequence: Synthetic peptide
GKPSSDDEATADSQHSTPPKKKERKVED

SEQ ID NO: 34 - protein, 21 aa, synthetic construct
  REGION 1..21: note = Description of Artificial Sequence: Synthetic peptide
GKPTADDQHSTPPKKKRKVED

SEQ ID NO: 35 - protein, 27 aa, synthetic construct
  REGION 1..27: note = Description of Artificial Sequence: Synthetic peptide
GGKRTADGSEFESPKKARKVEAYPKAK

SEQ ID NO: 36 - protein, 18 aa, synthetic construct
  REGION 1..18: note = Description of Artificial Sequence: Synthetic peptide
EKIRLRPGRKKRYRLKHL

SEQ ID NO: 37 - protein, 18 aa, synthetic construct
  REGION 1..18: note = Description of Artificial Sequence: Synthetic peptide
PEGTRQARRNRRRRWRKR

SEQ ID NO: 38 - protein, 18 aa, synthetic construct
  REGION 1..18: note = Description of Artificial Sequence: Synthetic peptide
PEGTRQPRRNRRRRWRKR

SEQ ID NO: 39 - protein, 26 aa, synthetic construct
  REGION 1..26: note = Description of Artificial Sequence: Synthetic peptide
GVKRSYGAARGDDRRRPNVVAPYKAW

SEQ ID NO: 40 - protein, 26 aa, synthetic construct
  REGION 1..26: note = Description of Artificial Sequence: Synthetic peptide
KSVPNRTRTYIKLKRLRFKGAPYKAW

SEQ ID NO: 41 - protein, 27 aa, synthetic construct
  REGION 1..27: note = Description of Artificial Sequence: Synthetic peptide
EMRRRREEEGLQLRKQKREEQLFKRRN

SEQ ID NO: 42 - protein, 14 aa, synthetic construct
  REGION 1..14: note = Description of Artificial Sequence: Synthetic peptide
FEAALAEALAEALA

SEQ ID NO: 43 - protein, 12 aa, synthetic construct
  REGION 1..12: note = Description of Artificial Sequence: Synthetic peptide
  REGION 1..12: note = N-term Ac
  REGION 1..12: note = C-term NHCH3
LARLLPRLLARL

SEQ ID NO: 44 - protein, 20 aa, synthetic construct
  REGION 1..20: note = Description of Artificial Sequence: Synthetic peptide
GLLEELLELLEELWEELLEG

SEQ ID NO: 45 - protein, 19 aa, synthetic construct
  REGION 1..19: note = Description of Artificial Sequence: Synthetic peptide
GWEGLIEGIEGGWEGLIEG

SEQ ID NO: 46 - protein, 20 aa, synthetic construct
  REGION 1..20: note = Description of Artificial Sequence: Synthetic peptide
GLFEALAEFIEGGWEGLIEG

SEQ ID NO: 47 - protein, 20 aa, synthetic construct
  REGION 1..20: note = Description of Artificial Sequence: Synthetic peptide
GLFEALLELLESLWELLLEA

SEQ ID NO: 48 - protein, 22 aa, synthetic construct
  REGION 1..22: note = Description of Artificial Sequence: Synthetic peptide
GGYCLEKWMIVASELKCFGNTA

SEQ ID NO: 49 - protein, 23 aa, synthetic construct
  REGION 1..23: note = Description of Artificial Sequence: Synthetic peptide
GGYCLTRWMLIEAELKCFGNTAV

SEQ ID NO: 50 - protein, 30 aa, synthetic construct
  REGION 1..30: note = Description of Artificial Sequence: Synthetic polypeptide
WEAALAEALAEALAEHLAEALAEALEALAA

SEQ ID NO: 51 - protein, 23 aa, synthetic construct
  REGION 1..23: note = Description of Artificial Sequence: Synthetic peptide
GLFGAIAGFIENGWEGMIDGWYG

SEQ ID NO: 52 - protein, 26 aa, synthetic construct
  REGION 1..26: note = Description of Artificial Sequence: Synthetic peptide
GIGAVLKVLTTGLPALISWIKRKRQQ

SEQ ID NO: 53 - protein, 13 aa, synthetic construct
  REGION 1..13: note = Description of Artificial Sequence: Synthetic peptide
GRKKRRQRRRPPQ

SEQ ID NO: 54 - protein, 16 aa, synthetic construct
  REGION 1..16: note = Description of Artificial Sequence: Synthetic peptide
RQIKIWFQNRRMKWKK

SEQ ID NO: 55 - protein, 27 aa, synthetic construct
  REGION 1..27: note = Description of Artificial Sequence: Synthetic peptide
GWTLNSAGYLLGKINLKALAALAKKIL

SEQ ID NO: 56 - protein, 30 aa, synthetic construct
  REGION 1..30: note = Description of Artificial Sequence: Synthetic polypeptide
WEAKLAKALAKALAKHLAKALAKALKACEA

SEQ ID NO: 57 - protein, 20 aa, synthetic construct
  REGION 1..20: note = Description of Artificial Sequence: Synthetic peptide
GLFKALLKLLKSLWKLLLKA

SEQ ID NO: 58 - protein, 20 aa, synthetic construct
  REGION 1..20: note = Description of Artificial Sequence: Synthetic peptide
GLFRALLRLLRSLWRLLLRA

SEQ ID NO: 59 - protein, 20 aa, synthetic construct
  REGION 1..20: note = Description of Artificial Sequence: Synthetic peptide
GLFEALLELLESLYELLLEA

SEQ ID NO: 60 - protein, 12 aa, synthetic construct
  REGION 1..12: note = Description of Artificial Sequence: Synthetic peptide
GLFEALEELWEA

SEQ ID NO: 61 - protein, 10 aa, synthetic construct
  REGION 1..10: note = Description of Artificial Sequence: Synthetic peptide
GLFLLEEWLE

SEQ ID NO: 62 - protein, 11 aa, synthetic construct
  REGION 1..11: note = Description of Artificial Sequence: Synthetic peptide
GLFLLEEWLEK

SEQ ID NO: 63 - protein, 21 aa, synthetic construct
  REGION 1..21: note = Description of Artificial Sequence: Synthetic peptide
GLFEALLELLESLWELLLEAK

SEQ ID NO: 64 - protein, 11 aa, synthetic construct
  REGION 1..11: note = Description of Artificial Sequence: Synthetic peptide
  REGION 1..11: note = N-term succinyl
GLFKLLEEWLE

SEQ ID NO: 65 - protein, 12 aa, synthetic construct
  REGION 1..12: note = Description of Artificial Sequence: Synthetic peptide
  REGION 1..12: note = N-term succinyl
GLFKLLEEWLEK

SEQ ID NO: 66 - protein, 20 aa, synthetic construct
  REGION 1..20: note = Description of Artificial Sequence: Synthetic peptide
GLFEAIAEFIEGGWEGLIEG

SEQ ID NO: 67 - protein, 20 aa, synthetic construct
  REGION 1..20: note = Description of Artificial Sequence: Synthetic peptide
GLFKAIAKFIKGGWKGLIKG

SEQ ID NO: 68 - protein, 17 aa, synthetic construct
  REGION 1..17: note = Description of Artificial Sequence: Synthetic peptide
IRFKKTKLIASIAMALC

SEQ ID NO: 69 - protein, 30 aa, synthetic construct
  REGION 1..30: note = Description of Artificial Sequence: Synthetic polypeptide
ALAGTIIAGASLTFQVLDKVLEELGKVSRK

SEQ ID NO: 70 - protein, 23 aa, synthetic construct
  REGION 1..23: note = Description of Artificial Sequence: Synthetic peptide
GLFEAIEGFIENGWEGMIDGWYG

SEQ ID NO: 71 - protein, 17 aa, synthetic construct
  REGION 1..17: note = Description of Artificial Sequence: Synthetic peptide
GYICRRARGDNPDDRCT

SEQ ID NO: 72 - protein, 22 aa, synthetic construct
  REGION 1..22: note = Description of Artificial Sequence: Synthetic peptide
GLFEAIAEFIEGGWEGLIEGCA

SEQ ID NO: 73 - protein, 24 aa, synthetic construct
  REGION 1..24: note = Description of Artificial Sequence: Synthetic peptide
GLFHAIAHFIHGGWHGLIHGWWYG

SEQ ID NO: 74 - protein, 30 aa, synthetic construct
  REGION 1..30: note = Description of Artificial Sequence: Synthetic polypeptide
RRRQRRKKRGGDIMGEWGNEIFGAIAGFLG

SEQ ID NO: 75 - protein, 22 aa, synthetic construct
  REGION 1..22: note = Description of Artificial Sequence: Synthetic peptide
GLFEAIADFIENGWEGMIDGGG

SEQ ID NO: 76 - protein, 31 aa, synthetic construct
  REGION 1..31: note = Description of Artificial Sequence: Synthetic polypeptide
ALAGTIIAGASLTFQVLDKVLEELGKVSRKK

SEQ ID NO: 77 - protein, 15 aa, synthetic construct
  REGION 1..15: note = Description of Artificial Sequence: Synthetic peptide
IRFKKTKLIASIAMA

SEQ ID NO: 78 - protein, 18 aa, synthetic construct
  REGION 1..18: note = Description of Artificial Sequence: Synthetic peptide
GLWHLLLHLWRRLLRLLR

SEQ ID NO: 79 - protein, 21 aa, synthetic construct
  REGION 1..21: note = Description of Artificial Sequence: Synthetic peptide
KKIMLLLMTLLLVSLPLAQEQ

SEQ ID NO: 80 - protein, 23 aa, synthetic construct
  REGION 1..23: note = Description of Artificial Sequence: Synthetic peptide
GLFEALLELLESLWELLLEAWYG

SEQ ID NO: 81 - protein, 18 aa, synthetic construct
  REGION 1..18: note = Description of Artificial Sequence: Synthetic peptide
RLLRLLLRLWRRLLRLLR

SEQ ID NO: 82 - protein, 26 aa, synthetic construct
  REGION 1..26: note = Description of Artificial Sequence: Synthetic peptide
LLELELLELELLLELELLELELLLEL

SEQ ID NO: 83 - protein, 28 aa, synthetic construct
  REGION 1..28: note = Description of Artificial Sequence: Synthetic peptide
GLFEALLELLESLWELLLEARRRRRRRR

SEQ ID NO: 84 - protein, 26 aa, synthetic construct
  REGION 1..26: note = Description of Artificial Sequence: Synthetic peptide
GLFEALLELLESLWELLLEARRRRRR

SEQ ID NO: 85 - protein, 28 aa, synthetic construct
  REGION 1..28: note = Description of Artificial Sequence: Synthetic peptide
GLFEALLELLESLWELLLEAKKKKKKKK

SEQ ID NO: 86 - protein, 26 aa, synthetic construct
  REGION 1..26: note = Description of Artificial Sequence: Synthetic peptide
GLFEALLELLESLWELLLEAKKKKKK

SEQ ID NO: 87 - protein, 22 aa, synthetic construct
  REGION 1..22: note = Description of Artificial Sequence: Synthetic peptide
GLFEALLELLESLWELLLEAKK

SEQ ID NO: 88 - protein, 24 aa, synthetic construct
  REGION 1..24: note = Description of Artificial Sequence: Synthetic peptide
GLFEALLELLESLWELLLEAKKKK

SEQ ID NO: 89 - protein, 22 aa, synthetic construct
  REGION 1..22: note = Description of Artificial Sequence: Synthetic peptide
GLFEALLELLESLWELLLEAEE

SEQ ID NO: 90 - protein, 24 aa, synthetic construct
  REGION 1..24: note = Description of Artificial Sequence: Synthetic peptide
GLFEALLELLESLWELLLEAEEEE

SEQ ID NO: 91 - protein, 26 aa, synthetic construct
  REGION 1..26: note = Description of Artificial Sequence: Synthetic peptide
GLFEALLELLESLWELLLEAEEEEEE

SEQ ID NO: 92 - protein, 18 aa, synthetic construct
  REGION 1..18: note = Description of Artificial Sequence: Synthetic peptide
GLFEALLELLESLWELLL

SEQ ID NO: 93 - protein, 36 aa, synthetic construct
  REGION 1..36: note = Description of Artificial Sequence: Synthetic polypeptide
PLSSIFSRIGDPRGARRYAKMKRRRRRVARRHRRRP

SEQ ID NO: 94 - protein, 13 aa, synthetic construct
  REGION 1..13: note = Description of Artificial Sequence: Synthetic peptide
GPFHYFQFLFPPV

SEQ ID NO: 95 - protein, 13 aa, synthetic construct
  REGION 1..13: note = Description of Artificial Sequence: Synthetic peptide
GSSSWWQRWWPPW

SEQ ID NO: 96 - protein, 9 aa, synthetic construct
  REGION 1..9: note = Description of Artificial Sequence: Synthetic peptide
RRRQRRKKR

SEQ ID NO: 97 - protein, 4 aa, synthetic construct
  REGION 1..4: note = Description of Artificial Sequence: Synthetic peptide
KKKK

SEQ ID NO: 98 - protein, 6 aa, synthetic construct
  REGION 1..6: note = Description of Artificial Sequence: Synthetic peptide
KKKKKK

SEQ ID NO: 99 - protein, 8 aa, synthetic construct
  REGION 1..8: note = Description of Artificial Sequence: Synthetic peptide
KKKKKKKK

SEQ ID NO: 100 - protein, 10 aa, synthetic construct
  REGION 1..10: note = Description of Artificial Sequence: Synthetic peptide
KKKKKKKKKK

SEQ ID NO: 101 - protein, 12 aa, synthetic construct
  REGION 1..12: note = Description of Artificial Sequence: Synthetic peptide
KKKKKKKKKKKK

SEQ ID NO: 102 - protein, 16 aa, synthetic construct
  REGION 1..16: note = Description of Artificial Sequence: Synthetic peptide
KKKKKKKKKKKKKKKK

SEQ ID NO: 103 - protein, 20 aa, synthetic construct
  REGION 1..20: note = Description of Artificial Sequence: Synthetic peptide
KKKKKKKKKKKKKKKKKKKK

SEQ ID NO: 104 - protein, 24 aa, synthetic construct
  REGION 1..24: note = Description of Artificial Sequence: Synthetic peptide
KKKKKKKKKKKKKKKKKKKKKKKK

SEQ ID NO: 105 - protein, 4 aa, synthetic construct
  REGION 1..4: note = Description of Artificial Sequence: Synthetic peptide
RRRR

SEQ ID NO: 106 - protein, 6 aa, synthetic construct
  REGION 1..6: note = Description of Artificial Sequence: Synthetic peptide
RRRRRR

SEQ ID NO: 107 - protein, 8 aa, synthetic construct
  REGION 1..8: note = Description of Artificial Sequence: Synthetic peptide
RRRRRRRR

SEQ ID NO: 108 - protein, 10 aa, synthetic construct
  REGION 1..10: note = Description of Artificial Sequence: Synthetic peptide
RRRRRRRRRR

SEQ ID NO: 109 - protein, 12 aa, synthetic construct
  REGION 1..12: note = Description of Artificial Sequence: Synthetic peptide
RRRRRRRRRRRR

SEQ ID NO: 110 - protein, 16 aa, synthetic construct
  REGION 1..16: note = Description of Artificial Sequence: Synthetic peptide
RRRRRRRRRRRRRRRR

SEQ ID NO: 111 - protein, 20 aa, synthetic construct
  REGION 1..20: note = Description of Artificial Sequence: Synthetic peptide
RRRRRRRRRRRRRRRRRRRR

SEQ ID NO: 112 - protein, 24 aa, synthetic construct
  REGION 1..24: note = Description of Artificial Sequence: Synthetic peptide
RRRRRRRRRRRRRRRRRRRRRRRR

SEQ ID NO: 113 - skipped

SEQ ID NO: 114 - protein, 25 aa, synthetic construct
  REGION 1..25: note = Description of Artificial Sequence: Synthetic peptide
KKKKKKKKWKGGGGACYGLPHLFCG

SEQ ID NO: 115 - protein, 13 aa, synthetic construct
  REGION 1..13: note = Description of Artificial Sequence: Synthetic peptide
YKAKKKKKKKKWK

SEQ ID NO: 116 - protein, 18 aa, synthetic construct
  REGION 1..18: note = Description of Artificial Sequence: Synthetic peptide
KTPKKAKKPKTPKKAKKP

SEQ ID NO: 117 - protein, 29 aa, synthetic construct
  REGION 1..29: note = Description of Artificial Sequence: Synthetic peptide
KKAKKPAATRKSSKNPKKPKTVKPKKVAK

SEQ ID NO: 118 - protein, 24 aa, synthetic construct
  REGION 1..24: note = Description of Artificial Sequence: Synthetic peptide
RGARRYSKMKRRRRRVARRHRRRP

SEQ ID NO: 119 - protein, 22 aa, synthetic construct
  REGION 1..22: note = Description of Artificial Sequence: Synthetic peptide
TRQARRNRRRRWRERQRGSGSG

SEQ ID NO: 120 - protein, 29 aa, synthetic construct
  REGION 1..29: note = Description of Artificial Sequence: Synthetic peptide
KRPRGRPKGSKKNWRRRKRRASRRSPRRR

SEQ ID NO: 121 - protein, 34 aa, synthetic construct
  REGION 1..34: note = Description of Artificial Sequence: Synthetic polypeptide
KRGRGRPRKQPPKEPSEVPTPKRPRGRPKGSKNK

SEQ ID NO: 122 - protein, 33 aa, synthetic construct
  REGION 1..33: note = Description of Artificial Sequence: Synthetic polypeptide
KEKYEKDIAAYRAKGKPAAKKGVVKAEKSKKKK

SEQ ID NO: 123 - protein, 15 aa, synthetic construct
  REGION 1..15: note = Description of Artificial Sequence: Synthetic peptide
YKAKKKKKKKKKKWK

SEQ ID NO: 124 - protein, 10 aa, synthetic construct
  REGION 1..10: note = Description of Artificial Sequence: Synthetic peptide
KKKKKKKGGC

SEQ ID NO: 125 - protein, 13 aa, synthetic construct
  REGION 1..13: note = Description of Artificial Sequence: Synthetic peptide
YRARRRRRRRRWR

SEQ ID NO: 126 - protein, 15 aa, synthetic construct
  REGION 1..15: note = Description of Artificial Sequence: Synthetic peptide
YRARRRRRRRRRRWR

SEQ ID NO: 127 - protein, 42 aa, synthetic construct
  REGION 1..42: note = Description of Artificial Sequence: Synthetic polypeptide
KGDPKKPRGKMSSYAFFVQTCREEHKKKHPDASVNFSEFSKK

SEQ ID NO: 128 - protein, 16 aa, synthetic construct
  REGION 1..16: note = Description of Artificial Sequence: Synthetic peptide
KKQLKKQLKKQLKQWK

SEQ ID NO: 129 - protein, 16 aa, synthetic construct
  REGION 1..16: note = Description of Artificial Sequence: Synthetic peptide
KKSPKKSPKKSPKKSK

SEQ ID NO: 130 - protein, 13 aa, synthetic construct
  REGION 1..13: note = Description of Artificial Sequence: Synthetic peptide
KLSKLEKKSKLEK

SEQ ID NO: 131 - protein, 20 aa, synthetic construct
  REGION 1..20: note = Description of Artificial Sequence: Synthetic peptide
KLSKLEKKLSKLEKKSKLEK

SEQ ID NO: 132 - protein, 15 aa, synthetic construct
  REGION 1..15: note = Description of Artificial Sequence: Synthetic peptide
KSLKKSLKKSLKKSK

SEQ ID NO: 133 - protein, 22 aa, synthetic construct
  REGION 1..22: note = Description of Artificial Sequence: Synthetic peptide
KIRRRGKNKVAARTCRQRRTDR

SEQ ID NO: 134 - protein, 22 aa, synthetic construct
  REGION 1..22: note = Description of Artificial Sequence: Synthetic peptide
KIRRRGKNKVAAQNCRKRKLET

SEQ ID NO: 135 - protein, 24 aa, synthetic construct
  REGION 1..24: note = Description of Artificial Sequence: Synthetic peptide
KRRIRREKNKMAAAKCRNRRRELT

SEQ ID NO: 136 - protein, 12 aa, synthetic construct
  REGION 1..12: note = Description of Artificial Sequence: Synthetic peptide
KDRSNLLERHTR

SEQ ID NO: 137 - protein, 16 aa, synthetic construct
  REGION 1..16: note = Description of Artificial Sequence: Synthetic peptide
KRPAATKKAGQAKKKL

SEQ ID NO: 138 - protein, 10 aa, synthetic construct
  REGION 1..10: note = Description of Artificial Sequence: Synthetic peptide
RRRRRREEEE

SEQ ID NO: 139 - protein, 12 aa, synthetic construct
  REGION 1..12: note = Description of Artificial Sequence: Synthetic peptide
RRRRRREEEEEE

SEQ ID NO: 140 - protein, 14 aa, synthetic construct
  REGION 1..14: note = Description of Artificial Sequence: Synthetic peptide
RRRRRREEEEEEEE

SEQ ID NO: 141 - protein, 12 aa, synthetic construct
  REGION 1..12: note = Description of Artificial Sequence: Synthetic peptide
RRRRRRRREEEE

SEQ ID NO: 142 - protein, 14 aa, synthetic construct
  REGION 1..14: note = Description of Artificial Sequence: Synthetic peptide
RRRRRRRREEEEEE

SEQ ID NO: 143 - protein, 16 aa, synthetic construct
  REGION 1..16: note = Description of Artificial Sequence: Synthetic peptide
RRRRRRRREEEEEEEE

SEQ ID NO: 144 - protein, 16 aa, synthetic construct
  REGION 1..16: note = Description of Artificial Sequence: Synthetic peptide
RRRRRRRRRRRREEEE

SEQ ID NO: 145 - protein, 18 aa, synthetic construct
  REGION 1..18: note = Description of Artificial Sequence: Synthetic peptide
RRRRRRRRRRRREEEEEE

SEQ ID NO: 146 - protein, 18 aa, synthetic construct
  REGION 1..18: note = Description of Artificial Sequence: Synthetic peptide
RRRRRRRRRRRREEEEEE

SEQ ID NO: 147 - protein, 8 aa, synthetic construct
  REGION 1..8: note = Description of Artificial Sequence: Synthetic peptide
KLSKLEKK

SEQ ID NO: 148 - protein, 5 aa, synthetic construct
  REGION 1..5: note = Description of Artificial Sequence: Synthetic peptide
SKLEK

SEQ ID NO: 149 - protein, 15 aa, synthetic construct
  REGION 1..15: note = Description of Artificial Sequence: Synthetic peptide
KLSKLEKKLSKLEKK

SEQ ID NO: 150 - protein, 15 aa, synthetic construct
  REGION 1..15: note = Description of Artificial Sequence: Synthetic peptide
PKKKRKVGGGRGDSP

SEQ ID NO: 151 - protein, 9 aa, synthetic construct
  REGION 1..9: note = Description of Artificial Sequence: Synthetic peptide
LPHKSMPCG

SEQ ID NO: 152 - protein, 12 aa, synthetic construct
  REGION 1..12: note = Description of Artificial Sequence: Synthetic peptide
GACLQHKSMPCG

SEQ ID NO: 153 - protein, 9 aa, synthetic construct
  REGION 1..9: note = Description of Artificial Sequence: Synthetic peptide
YGLPHLFCG

SEQ ID NO: 154 - protein, 9 aa, synthetic construct
  REGION 1..9: note = Description of Artificial Sequence: Synthetic peptide
SERSMNFCG

SEQ ID NO: 155 - protein, 15 aa, synthetic construct
  REGION 1..15: note = Description of Artificial Sequence: Synthetic peptide
DHYSLYEDLERGTDK

SEQ ID NO: 156 - protein, 16 aa, synthetic construct
  REGION 1..16: note = Description of Artificial Sequence: Synthetic peptide
ISLPRTSGAQRASTTR

SEQ ID NO: 157 - protein, 17 aa, synthetic construct
  REGION 1..17: note = Description of Artificial Sequence: Synthetic peptide
EKLQTKYGLPHKVEFCG

SEQ ID NO: 158 - protein, 12 aa, synthetic construct
  REGION 1..12: note = Description of Artificial Sequence: Synthetic peptide
TRISESQAKPGD

SEQ ID NO: 159 - protein, 6 aa, synthetic construct
  REGION 1..6: note = Description of Artificial Sequence: Synthetic peptide
LVFFDY

SEQ ID NO: 160 - protein, 21 aa, synthetic construct
  REGION 1..21: note = Description of Artificial Sequence: Synthetic peptide
WGGNGPTTFDCSGYTKYVFAK

SEQ ID NO: 161 - protein, 15 aa, synthetic construct
  REGION 1..15: note = Description of Artificial Sequence: Synthetic peptide
INIGTTGWGDHYSLY

SEQ ID NO: 162 - protein, 6 aa, synthetic construct
  REGION 1..6: note = Description of Artificial Sequence: Synthetic peptide
YDNIHG

SEQ ID NO: 163 - protein, 13 aa, synthetic construct
  REGION 1..13: note = Description of Artificial Sequence: Synthetic peptide
AGWGKFLVGFGRV

SEQ ID NO: 164 - protein, 7 aa, synthetic construct
  REGION 1..7: note = Description of Artificial Sequence: Synthetic peptide
SIGYPLP

SEQ ID NO: 165 - protein, 8 aa, synthetic construct
  REGION 1..8: note = Description of Artificial Sequence: Synthetic peptide
TTHWGFTL

SEQ ID NO: 166 - protein, 12 aa, synthetic construct
  REGION 1..12: note = Description of Artificial Sequence: Synthetic peptide
HLQIQPYPQISG

SEQ ID NO: 167 - protein, 9 aa, synthetic construct
  REGION 1..9: note = Description of Artificial Sequence: Synthetic peptide
KLNIVSVNG

SEQ ID NO: 168 - skipped

SEQ ID NO: 169 - protein, 11 aa, synthetic construct
  REGION 1..11: note = Description of Artificial Sequence: Synthetic peptide
DNRIRLQAKAA

SEQ ID NO: 170 - protein, 10 aa, synthetic construct
  REGION 1..10: note = Description of Artificial Sequence: Synthetic peptide
KIKMVISWKG

SEQ ID NO: 171 - protein, 12 aa, synthetic construct
  REGION 1..12: note = Description of Artificial Sequence: Synthetic peptide
LPWYSYLYAVSA

SEQ ID NO: 172 - protein, 12 aa, synthetic construct
  REGION 1..12: note = Description of Artificial Sequence: Synthetic peptide
WNLPWYYSVSPT

SEQ ID NO: 173 - skipped

SEQ ID NO: 174 - protein, 9 aa, synthetic construct
  REGION 1..9: note = Description of Artificial Sequence: Synthetic peptide
PWYYSVSPT

SEQ ID NO: 175 - protein, 14 aa, synthetic construct
  REGION 1..14: note = Description of Artificial Sequence: Synthetic peptide
SSWESYKSGGGTRL

SEQ ID NO: 176 - protein, 18 aa, synthetic construct
  REGION 1..18: note = Description of Artificial Sequence: Synthetic peptide
RDWSSQHPGRCNGETHLK

SEQ ID NO: 177 - protein, 7 aa, synthetic construct
  REGION 1..7: note = Description of Artificial Sequence: Synthetic peptide
SLPTLTL

SEQ ID NO: 178 - protein, 21 aa, synthetic construct
  REGION 1..21: note = Description of Artificial Sequence: Synthetic peptide
VICTGGDYSFALPVGQWPVMT

SEQ ID NO: 179 - protein, 23 aa, synthetic construct
  REGION 1..23: note = Description of Artificial Sequence: Synthetic peptide
DKPSYQFGGHNSVDFEEDTLPKV

SEQ ID NO: 180 - protein, 26 aa, synthetic construct
  REGION 1..26: note = Description of Artificial Sequence: Synthetic peptide
RARRRKRASATQLYQTCKASGTCPPD

SEQ ID NO: 181 - protein, 18 aa, synthetic construct
  REGION 1..18: note = Description of Artificial Sequence: Synthetic peptide
SGDYSFALPVGQWPWMTG

SEQ ID NO: 182 - protein, 17 aa, synthetic construct
  REGION 1..17: note = Description of Artificial Sequence: Synthetic peptide
CTGGDYSFALPVGQWPW

SEQ ID NO: 183 - protein, 15 aa, synthetic construct
  REGION 1..15: note = Description of Artificial Sequence: Synthetic peptide
FYYDYDFFFDYWGQG

SEQ ID NO: 184 - protein, 15 aa, synthetic construct
  REGION 1..15: note = Description of Artificial Sequence: Synthetic peptide
HLRRLRRRLLREAEG

SEQ ID NO: 185 - protein, 17 aa, synthetic construct
  REGION 1..17: note = Description of Artificial Sequence: Synthetic peptide
DYYCAAWDDSLNGYSVF

SEQ ID NO: 186 - protein, 15 aa, synthetic construct
  REGION 1..15: note = Description of Artificial Sequence: Synthetic peptide
YYCLQSMEDPYTFGG

SEQ ID NO: 187 - protein, 20 aa, synthetic construct
  REGION 1..20: note = Description of Artificial Sequence: Synthetic peptide
YYCARSDGNYGYYYALDYDY

SEQ ID NO: 188 - protein, 12 aa, synthetic construct
  REGION 1..12: note = Description of Artificial Sequence: Synthetic peptide
AARSPSYYRYDY

SEQ ID NO: 189 - protein, 9 aa, synthetic construct
  REGION 1..9: note = Description of Artificial Sequence: Synthetic peptide
GPYYAMDYD

SEQ ID NO: 190 - protein, 22 aa, synthetic construct
  REGION 1..22: note = Description of Artificial Sequence: Synthetic peptide
YYCQQRSSYPYTEGGAYPKAWK

SEQ ID NO: 191 - protein, 18 aa, synthetic construct
  REGION 1..18: note = Description of Artificial Sequence: Synthetic peptide
YYCQRYDSDWSFGQGTKL

SEQ ID NO: 192 - protein, 18 aa, synthetic construct
  REGION 1..18: note = Description of Artificial Sequence: Synthetic peptide
YYCARSGYYAMDYWGQGT

SEQ ID NO: 193 - protein, 13 aa, synthetic construct
  REGION 1..13: note = Description of Artificial Sequence: Synthetic peptide
RVRRGACRGDCLG

SEQ ID NO: 194 - protein, 13 aa, synthetic construct
  REGION 1..13: note = Description of Artificial Sequence: Synthetic peptide
RVRRGACRYDCLG

SEQ ID NO: 195 - protein, 23 aa, synthetic construct
  REGION 1..23: note = Description of Artificial Sequence: Synthetic peptide
YYCAKGTHWGFWSGYFDYWGQGT

SEQ ID NO: 196 - protein, 17 aa, synthetic construct
  REGION 1..17: note = Description of Artificial Sequence: Synthetic peptide
GRENYHGCTTHWGFTLC

SEQ ID NO: 197 - protein, 17 aa, synthetic construct
  REGION 1..17: note = Description of Artificial Sequence: Synthetic peptide
VQATQSNQHTPRGGGSK

SEQ ID NO: 198 - protein, 7 aa, synthetic construct
  REGION 1..7: note = Description of Artificial Sequence: Synthetic peptide
DPRAPGS

SEQ ID NO: 199 - protein, 15 aa, synthetic construct
  REGION 1..15: note = Description of Artificial Sequence: Synthetic peptide
YYCQQRSSYPYTFGG

SEQ ID NO: 200 - protein, 21 aa, synthetic construct
  REGION 1..21: note = Description of Artificial Sequence: Synthetic peptide
AARSPSYYRYDYGPYYAMDYD

SEQ ID NO: 201 - protein, 18 aa, synthetic construct
  REGION 1..18: note = Description of Artificial Sequence: Synthetic peptide
GPKLTGILISILSLFVES

SEQ ID NO: 202 - protein, 11 aa, synthetic construct
  REGION 1..11: note = Description of Artificial Sequence: Synthetic peptide
KYILRWRPKNS

SEQ ID NO: 203 - protein, 5 aa, synthetic construct
  REGION 1..5: note = Description of Artificial Sequence: Synthetic peptide
IKVAV

SEQ ID NO: 204 - protein, 20 aa, synthetic construct
  REGION 1..20: note = Description of Artificial Sequence: Synthetic peptide
WTPPRAQITGYRLTVGLTRR

SEQ ID NO: 205 - protein, 12 aa, synthetic construct
  REGION 1..12: note = Description of Artificial Sequence: Synthetic peptide
AASIKVAVSADR

SEQ ID NO: 206 - protein, 6 aa, synthetic construct
  REGION 1..6: note = Description of Artificial Sequence: Synthetic peptide
KLDAPT

SEQ ID NO: 207 - protein, 12 aa, synthetic construct
  REGION 1..12: note = Description of Artificial Sequence: Synthetic peptide
NRWHSIYITRFG

SEQ ID NO: 208 - protein, 5 aa, synthetic construct
  REGION 1..5: note = Description of Artificial Sequence: Synthetic peptide
PHSRN

SEQ ID NO: 209 - protein, 12 aa, synthetic construct
  REGION 1..12: note = Description of Artificial Sequence: Synthetic peptide
SSFHFDGSGYAM

SEQ ID NO: 210 - protein, 4 aa, synthetic construct
  REGION 1..4: note = Description of Artificial Sequence: Synthetic peptide
RGDS

SEQ ID NO: 211 - protein, 6 aa, synthetic construct
  REGION 1..6: note = Description of Artificial Sequence: Synthetic peptide
IAFQRN

SEQ ID NO: 212 - protein, 6 aa, synthetic construct
  REGION 1..6: note = Description of Artificial Sequence: Synthetic peptide
GRGDSP

SEQ ID NO: 213 - protein, 11 aa, synthetic construct
  REGION 1..11: note = Description of Artificial Sequence: Synthetic peptide
TWYKIAFQRRK

SEQ ID NO: 214 - protein, 7 aa, synthetic construct
  REGION 1..7: note = Description of Artificial Sequence: Synthetic peptide
EDGIHEL

SEQ ID NO: 215 - protein, 12 aa, synthetic construct
  REGION 1..12: note = Description of Artificial Sequence: Synthetic peptide
SLVRNRRVITIQ

SEQ ID NO: 216 - protein, 16 aa, synthetic construct
  REGION 1..16: note = Description of Artificial Sequence: Synthetic peptide
YRVRVTPKEKTGPMKE

SEQ ID NO: 217 - protein, 7 aa, synthetic construct
  REGION 1..7: note = Description of Artificial Sequence: Synthetic peptide
LQVQLSR

SEQ ID NO: 218 - protein, 9 aa, synthetic construct
  REGION 1..9: note = Description of Artificial Sequence: Synthetic peptide
SPPRRARVT

SEQ ID NO: 219 - protein, 12 aa, synthetic construct
  REGION 1..12: note = Description of Artificial Sequence: Synthetic peptide
RKRLQVQLSIRT

SEQ ID NO: 220 - protein, 9 aa, synthetic construct
  REGION 1..9: note = Description of Artificial Sequence: Synthetic peptide
ATETTITIS

SEQ ID NO: 221 - protein, 12 aa, synthetic construct
  REGION 1..12: note = Description of Artificial Sequence: Synthetic peptide
NAPFPKLSWTIQ

SEQ ID NO: 222 - protein, 31 aa, synthetic construct
  REGION 1..31: note = Description of Artificial Sequence: Synthetic polypeptide
VSPPRRARVTDATETTITISWRTKTETITGG

SEQ ID NO: 223 - protein, 12 aa, synthetic construct
  REGION 1..12: note = Description of Artificial Sequence: Synthetic peptide
WTIQTTVDRGLL

SEQ ID NO: 224 - protein, 11 aa, synthetic construct
  REGION 1..11: note = Description of Artificial Sequence: Synthetic peptide
KPDVRSYTITG

SEQ ID NO: 225 - protein, 13 aa, synthetic construct
  REGION 1..13: note = Description of Artificial Sequence: Synthetic peptide
DTINNGRDHMILI

SEQ ID NO: 226 - protein, 12 aa, synthetic construct
  REGION 1..12: note = Description of Artificial Sequence: Synthetic peptide
ANGQTPIQRYIK

SEQ ID NO: 227 - protein, 13 aa, synthetic construct
  REGION 1..13: note = Description of Artificial Sequence: Synthetic peptide
MILISIGKSQKRM

SEQ ID NO: 228 - protein, 30 aa, synthetic construct
  REGION 1..30: note = Description of Artificial Sequence: Synthetic polypeptide
PRARITGYIIKYEKPGSPPREVVPRPRPGV

SEQ ID NO: 229 - protein, 12 aa, synthetic construct
  REGION 1..12: note = Description of Artificial Sequence: Synthetic peptide
PPFLMLLKGSTR

SEQ ID NO: 230 - protein, 8 aa, synthetic construct
  REGION 1..8: note = Description of Artificial Sequence: Synthetic peptide
WQPPRARI

SEQ ID NO: 231 - protein, 13 aa, synthetic construct
  REGION 1..13: note = Description of Artificial Sequence: Synthetic peptide
NQRLASFSNAQQS

SEQ ID NO: 232 - protein, 19 aa, synthetic construct
  REGION 1..19: note = Description of Artificial Sequence: Synthetic peptide
WQPPRARITGYIIKYEKPG

SEQ ID NO: 233 - protein, 17 aa, synthetic construct
  REGION 1..17: note = Description of Artificial Sequence: Synthetic peptide
ISNVFVQRMSQSPEVLD

SEQ ID NO: 234 - protein, 19 aa, synthetic construct
  REGION 1..19: note = Description of Artificial Sequence: Synthetic peptide
YEKPGSPPREVVPRPRPGV

SEQ ID NO: 235 - protein, 13 aa, synthetic construct
  REGION 1..13: note = Description of Artificial Sequence: Synthetic peptide
KARSFNVNQLLQD

SEQ ID NO: 236 - protein, 15 aa, synthetic construct
  REGION 1..15: note = Description of Artificial Sequence: Synthetic peptide
KNNQKSEPLIGRKKT

SEQ ID NO: 237 - protein, 12 aa, synthetic construct
  REGION 1..12: note = Description of Artificial Sequence: Synthetic peptide
KNSFMALYLSKG

SEQ ID NO: 238 - protein, 8 aa, synthetic construct
  REGION 1..8: note = Description of Artificial Sequence: Synthetic peptide
EILDVPST

SEQ ID NO: 239 - protein, 19 aa, synthetic construct
  REGION 1..19: note = Description of Artificial Sequence: Synthetic peptide
KNSFMALYLSKGRLVFALG

SEQ ID NO: 240 - protein, 5 aa, synthetic construct
  REGION 1..5: note = Description of Artificial Sequence: Synthetic peptide
IDAPS

SEQ ID NO: 241 - protein, 20 aa, synthetic construct
  REGION 1..20: note = Description of Artificial Sequence: Synthetic peptide
RDSFVALYLSEGHVIFAGLG

SEQ ID NO: 242 - protein, 15 aa, synthetic construct
  REGION 1..15: note = Description of Artificial Sequence: Synthetic peptide
VVIDASTAIDAPSNL

SEQ ID NO: 243 - protein, 12 aa, synthetic construct
  REGION 1..12: note = Description of Artificial Sequence: Synthetic peptide
KPRLQFSLDIQT

SEQ ID NO: 244 - protein, 5 aa, synthetic construct
  REGION 1..5: note = Description of Artificial Sequence: Synthetic peptide
LDVPS

SEQ ID NO: 245 - protein, 12 aa, synthetic construct
  REGION 1..12: note = Description of Artificial Sequence: Synthetic peptide
DGQWHSVTVSIK

SEQ ID NO: 246 - protein, 4 aa, synthetic construct
  REGION 1..4: note = Description of Artificial Sequence: Synthetic peptide
REDV

SEQ ID NO: 247 - protein, 12 aa, synthetic construct
  REGION 1..12: note = Description of Artificial Sequence: Synthetic peptide
FVLYLGSKNAKK

SEQ ID NO: 248 - protein, 10 aa, synthetic construct
  REGION 1..10: note = Description of Artificial Sequence: Synthetic peptide
PHSRNRGDSP

SEQ ID NO: 249 - protein, 12 aa, synthetic construct
  REGION 1..12: note = Description of Artificial Sequence: Synthetic peptide
LAIKNDNLVYVY

SEQ ID NO: 250 - protein, 13 aa, synthetic construct
  REGION 1..13: note = Description of Artificial Sequence: Synthetic peptide
LWVTVRSQQRGLF

SEQ ID NO: 251 - protein, 12 aa, synthetic construct
  REGION 1..12: note = Description of Artificial Sequence: Synthetic peptide
AYFSIVKIERVG

SEQ ID NO: 252 - protein, 13 aa, synthetic construct
  REGION 1..13: note = Description of Artificial Sequence: Synthetic peptide
GTNNWWQSPSIQN

SEQ ID NO: 253 - protein, 12 aa, synthetic construct
  REGION 1..12: note = Description of Artificial Sequence: Synthetic peptide
DVISLYNFKHIY

SEQ ID NO: 254 - protein, 13 aa, synthetic construct
  REGION 1..13: note = Description of Artificial Sequence: Synthetic peptide
WVTVTLDLRQVFQ

SEQ ID NO: 255 - protein, 12 aa, synthetic construct
  REGION 1..12: note = Description of Artificial Sequence: Synthetic peptide
FFDGSSYAVVRD

SEQ ID NO: 256 - protein, 13 aa, synthetic construct
  REGION 1..13: note = Description of Artificial Sequence: Synthetic peptide
RQVFQVAYIIIKA

SEQ ID NO: 257 - protein, 14 aa, synthetic construct
  REGION 1..14: note = Description of Artificial Sequence: Synthetic peptide
LHVFYDFGFGFSNG

SEQ ID NO: 258 - protein, 14 aa, synthetic construct
  REGION 1..14: note = Description of Artificial Sequence: Synthetic peptide
LTRYKITPRRGPPT

SEQ ID NO: 259 - protein, 20 aa, synthetic construct
  REGION 1..20: note = Description of Artificial Sequence: Synthetic peptide
LKKAQINDAKYREISIIYHN

SEQ ID NO: 260 - protein, 12 aa, synthetic construct
  REGION 1..12: note = Description of Artificial Sequence: Synthetic peptide
LLEFTSARYIRL

SEQ ID NO: 261 - protein, 12 aa, synthetic construct
  REGION 1..12: note = Description of Artificial Sequence: Synthetic peptide
RAYFNGQSFIAS

SEQ ID NO: 262 - protein, 12 aa, synthetic construct
  REGION 1..12: note = Description of Artificial Sequence: Synthetic peptide
YIRLRLQRIRTL

SEQ ID NO: 263 - protein, 12 aa, synthetic construct
  REGION 1..12: note = Description of Artificial Sequence: Synthetic peptide
SRLRGKNPTKGK

SEQ ID NO: 264 - protein, 12 aa, synthetic construct
  REGION 1..12: note = Description of Artificial Sequence: Synthetic peptide
RRYYYSIKDISV

SEQ ID NO: 265 - protein, 12 aa, synthetic construct
  REGION 1..12: note = Description of Artificial Sequence: Synthetic peptide
LHKKGKNSSKPK

SEQ ID NO: 266 - protein, 12 aa, synthetic construct
  REGION 1..12: note = Description of Artificial Sequence: Synthetic peptide
SINNTAVNQRLT

SEQ ID NO: 267 - protein, 12 aa, synthetic construct
  REGION 1..12: note = Description of Artificial Sequence: Synthetic peptide
RLKTRSSHGMIF

SEQ ID NO: 268 - protein, 12 aa, synthetic construct
  REGION 1..12: note = Description of Artificial Sequence: Synthetic peptide
GGFLKYTVSYDI

SEQ ID NO: 269 - protein, 12 aa, synthetic construct
  REGION 1..12: note = Description of Artificial Sequence: Synthetic peptide
GEKSQFSIRLKT

SEQ ID NO: 270 - protein, 12 aa, synthetic construct
  REGION 1..12: note = Description of Artificial Sequence: Synthetic peptide
RDQLMTVLANVT

SEQ ID NO: 271 - protein, 12 aa, synthetic construct
  REGION 1..12: note = Description of Artificial Sequence: Synthetic peptide
TLFLAHGRLVFM

SEQ ID NO: 272 - protein, 12 aa, synthetic construct
  REGION 1..12: note = Description of Artificial Sequence: Synthetic peptide
ANVTHLLIRANY

SEQ ID NO: 273 - protein, 12 aa, synthetic construct
  REGION 1..12: note = Description of Artificial Sequence: Synthetic peptide
LVFMFNVGHKKL

SEQ ID NO: 274 - protein, 13 aa, synthetic construct
  REGION 1..13: note = Description of Artificial Sequence: Synthetic peptide
AGTFALRGDNPQG

SEQ ID NO: 275 - protein, 20 aa, synthetic construct
  REGION 1..20: note = Description of Artificial Sequence: Synthetic peptide
TLFLAHGRLVFMFNVGHKKL

SEQ ID NO: 276 - protein, 12 aa, synthetic construct
  REGION 1..12: note = Description of Artificial Sequence: Synthetic peptide
VLIKGGRARKHV

SEQ ID NO: 277 - protein, 19 aa, synthetic construct
  REGION 1..19: note = Description of Artificial Sequence: Synthetic peptide
DFMTLFLAHGRLVFMGNVG

SEQ ID NO: 278 - protein, 11 aa, synthetic construct
  REGION 1..11: note = Description of Artificial Sequence: Synthetic peptide
LSNIDYLIKAS

SEQ ID NO: 279 - protein, 12 aa, synthetic construct
  REGION 1..12: note = Description of Artificial Sequence: Synthetic peptide
HKKLKIRSQEKY

SEQ ID NO: 280 - protein, 12 aa, synthetic construct
  REGION 1..12: note = Description of Artificial Sequence: Synthetic peptide
LQQSRIANISME

SEQ ID NO: 281 - protein, 12 aa, synthetic construct
  REGION 1..12: note = Description of Artificial Sequence: Synthetic peptide
GAAWKIKGPIYL

SEQ ID NO: 282 - protein, 12 aa, synthetic construct
  REGION 1..12: note = Description of Artificial Sequence: Synthetic peptide
NLLLLLVKANLK

SEQ ID NO: 283 - protein, 12 aa, synthetic construct
  REGION 1..12: note = Description of Artificial Sequence: Synthetic peptide
VIRDSNVVQLDV

SEQ ID NO: 284 - protein, 12 aa, synthetic construct
  REGION 1..12: note = Description of Artificial Sequence: Synthetic peptide
HRDELLLWARKI

SEQ ID NO: 285 - protein, 12 aa, synthetic construct
  REGION 1..12: note = Description of Artificial Sequence: Synthetic peptide
GLIYYVAHQNQM

SEQ ID NO: 286 - protein, 12 aa, synthetic construct
  REGION 1..12: note = Description of Artificial Sequence: Synthetic peptide
KRRARDLVHRAE

SEQ ID NO: 287 - protein, 19 aa, synthetic construct
  REGION 1..19: note = Description of Artificial Sequence: Synthetic peptide
DYATLQLQEGRLHFMFDLG

SEQ ID NO: 288 - protein, 12 aa, synthetic construct
  REGION 1..12: note = Description of Artificial Sequence: Synthetic peptide
SQFQESVDNITK

SEQ ID NO: 289 - protein, 12 aa, synthetic construct
  REGION 1..12: note = Description of Artificial Sequence: Synthetic peptide
KKGSYNNIVVHV

SEQ ID NO: 290 - protein, 12 aa, synthetic construct
  REGION 1..12: note = Description of Artificial Sequence: Synthetic peptide
PGGMREKGRKAR

SEQ ID NO: 291 - protein, 12 aa, synthetic construct
  REGION 1..12: note = Description of Artificial Sequence: Synthetic peptide
ADNLLFYLGSAK

SEQ ID NO: 292 - protein, 12 aa, synthetic construct
  REGION 1..12: note = Description of Artificial Sequence: Synthetic peptide
MEMQANLLLDRL

SEQ ID NO: 293 - protein, 12 aa, synthetic construct
  REGION 1..12: note = Description of Artificial Sequence: Synthetic peptide
GSAKFIDFLAIE

SEQ ID NO: 294 - protein, 11 aa, synthetic construct
  REGION 1..11: note = Description of Artificial Sequence: Synthetic peptide
LSEIKLLISAR

SEQ ID NO: 295 - protein, 12 aa, synthetic construct
  REGION 1..12: note = Description of Artificial Sequence: Synthetic peptide
KVSFLWWVGSGV

SEQ ID NO: 296 - protein, 15 aa, synthetic construct
  REGION 1..15: note = Description of Artificial Sequence: Synthetic peptide
RDFTKATNIRLRFLR

SEQ ID NO: 297 - protein, 12 aa, synthetic construct
  REGION 1..12: note = Description of Artificial Sequence: Synthetic peptide
SYWYRIEASRTG

SEQ ID NO: 298 - protein, 12 aa, synthetic construct
  REGION 1..12: note = Description of Artificial Sequence: Synthetic peptide
ISTVMFKFRTFS

SEQ ID NO: 299 - protein, 12 aa, synthetic construct
  REGION 1..12: note = Description of Artificial Sequence: Synthetic peptide
YFDGTGFAKAVG

SEQ ID NO: 300 - protein, 13 aa, synthetic construct
  REGION 1..13: note = Description of Artificial Sequence: Synthetic peptide
KQANISIVDIDSN

SEQ ID NO: 301 - protein, 12 aa, synthetic construct
  REGION 1..12: note = Description of Artificial Sequence: Synthetic peptide
NGQWHKVTAKKI

SEQ ID NO: 302 - protein, 12 aa, synthetic construct
  REGION 1..12: note = Description of Artificial Sequence: Synthetic peptide
FSTRNESGIILL

SEQ ID NO: 303 - protein, 12 aa, synthetic construct
  REGION 1..12: note = Description of Artificial Sequence: Synthetic peptide
AKKIKNRLELVV

SEQ ID NO: 304 - protein, 12 aa, synthetic construct
  REGION 1..12: note = Description of Artificial Sequence: Synthetic peptide
RRQTTQAYYAIF

SEQ ID NO: 305 - protein, 14 aa, synthetic construct
  REGION 1..14: note = Description of Artificial Sequence: Synthetic peptide
GFPGGLNQFGLTTN

SEQ ID NO: 306 - protein, 12 aa, synthetic construct
  REGION 1..12: note = Description of Artificial Sequence: Synthetic peptide
YAIFLNKGRLEV

SEQ ID NO: 307 - protein, 13 aa, synthetic construct
  REGION 1..13: note = Description of Artificial Sequence: Synthetic peptide
NQFGLTTNIRFRG

SEQ ID NO: 308 - protein, 12 aa, synthetic construct
  REGION 1..12: note = Description of Artificial Sequence: Synthetic peptide
KNRLTIELEVRT

SEQ ID NO: 309 - protein, 13 aa, synthetic construct
  REGION 1..13: note = Description of Artificial Sequence: Synthetic peptide
IRSLKLTKGTGKP

SEQ ID NO: 310 - protein, 12 aa, synthetic construct
  REGION 1..12: note = Description of Artificial Sequence: Synthetic peptide
GLLFYMARINHA

SEQ ID NO: 311 - protein, 13 aa, synthetic construct
  REGION 1..13: note = Description of Artificial Sequence: Synthetic peptide
AKALELRGVQPVS

SEQ ID NO: 312 - protein, 12 aa, synthetic construct
  REGION 1..12: note = Description of Artificial Sequence: Synthetic peptide
VQLRNGFPYFSY

SEQ ID NO: 313 - protein, 13 aa, synthetic construct
  REGION 1..13: note = Description of Artificial Sequence: Synthetic peptide
GQLFHVAYILIKF

SEQ ID NO: 314 - protein, 12 aa, synthetic construct
  REGION 1..12: note = Description of Artificial Sequence: Synthetic peptide
HKIKIVRVKQEG

SEQ ID NO: 315 - protein, 12 aa, synthetic construct
  REGION 1..12: note = Description of Artificial Sequence: Synthetic peptide
NVLSLYNFKTTF

SEQ ID NO: 316 - protein, 19 aa, synthetic construct
  REGION 1..19: note = Description of Artificial Sequence: Synthetic peptide
DFGTVQLRNGFPFFSYDLG

SEQ ID NO: 317 - protein, 13 aa, synthetic construct
  REGION 1..13: note = Description of Artificial Sequence: Synthetic peptide
SQRIYQFAKLNYT

SEQ ID NO: 318 - protein, 12 aa, synthetic construct
  REGION 1..12: note = Description of Artificial Sequence: Synthetic peptide
NIRLRFLRTNTL

SEQ ID NO: 319 - protein, 13 aa, synthetic construct
  REGION 1..13: note = Description of Artificial Sequence: Synthetic peptide
EVNVTLDLGQVFH

SEQ ID NO: 320 - protein, 12 aa, synthetic construct
  REGION 1..12: note = Description of Artificial Sequence: Synthetic peptide
GKNTGDHFVLYM

SEQ ID NO: 321 - protein, 13 aa, synthetic construct
  REGION 1..13: note = Description of Artificial Sequence: Synthetic peptide
GQVFHVAYVLIKF

SEQ ID NO: 322 - protein, 13 aa, synthetic construct
  REGION 1..13: note = Description of Artificial Sequence: Synthetic peptide
VVSLYNFEQTFML

SEQ ID NO: 323 - protein, 21 aa, synthetic construct
  REGION 1..21: note = Description of Artificial Sequence: Synthetic peptide
HQQDLGTAGSCLRKFSTMFLF

SEQ ID NO: 324 - protein, 12 aa, synthetic construct
  REGION 1..12: note = Description of Artificial Sequence: Synthetic peptide
RFDQELRLVSYN

SEQ ID NO: 325 - protein, 24 aa, synthetic construct
  REGION 1..24: note = Description of Artificial Sequence: Synthetic peptide
HQQDLGTAGSCLRKFSTMFLFCNI

SEQ ID NO: 326 - protein, 13 aa, synthetic construct
  REGION 1..13: note = Description of Artificial Sequence: Synthetic peptide
RLVSYSGVLFFLK

SEQ ID NO: 327 - protein, 9 aa, synthetic construct
  REGION 1..9: note = Description of Artificial Sequence: Synthetic peptide
VAEIDGIEL

SEQ ID NO: 328 - protein, 12 aa, synthetic construct
  REGION 1..12: note = Description of Artificial Sequence: Synthetic peptide
NWRHISYITRFG

SEQ ID NO: 329 - protein, 6 aa, synthetic construct
  REGION 1..6: note = Description of Artificial Sequence: Synthetic peptide
GIIFFL

SEQ ID NO: 330 - protein, 12 aa, synthetic construct
  REGION 1..12: note = Description of Artificial Sequence: Synthetic peptide
KRLQVQLRSIRT

SEQ ID NO: 331 - protein, 12 aa, synthetic construct
  REGION 1..12: note = Description of Artificial Sequence: Synthetic peptide
ASKAIQVFLLGG

SEQ ID NO: 332 - protein, 12 aa, synthetic construct
  REGION 1..12: note = Description of Artificial Sequence: Synthetic peptide
TWYKIAFQRNRK

SEQ ID NO: 333 - protein, 12 aa, synthetic construct
  REGION 1..12: note = Description of Artificial Sequence: Synthetic peptide
VLVRVERATVFS

SEQ ID NO: 334 - protein, 12 aa, synthetic construct
  REGION 1..12: note = Description of Artificial Sequence: Synthetic peptide
QVFQVAYIIIKA

SEQ ID NO: 335 - protein, 12 aa, synthetic construct
  REGION 1..12: note = Description of Artificial Sequence: Synthetic peptide
TVFSVDQDNMLE

SEQ ID NO: 336 - protein, 13 aa, synthetic construct
  REGION 1..13: note = Description of Artificial Sequence: Synthetic peptide
GEFYFDLRLKGDK

SEQ ID NO: 337 - protein, 12 aa, synthetic construct
  REGION 1..12: note = Description of Artificial Sequence: Synthetic peptide
RLRGPQRVFDLH

SEQ ID NO: 338 - protein, 9 aa, synthetic construct
  REGION 1..9: note = Description of Artificial Sequence: Synthetic peptide
GTPGPQGIA

SEQ ID NO: 339 - protein, 11 aa, synthetic construct
  REGION 1..11: note = Description of Artificial Sequence: Synthetic peptide
FDLHQNMGSVN

SEQ ID NO: 340 - protein, 6 aa, synthetic construct
  REGION 1..6: note = Description of Artificial Sequence: Synthetic peptide
GQRDVV

SEQ ID NO: 341 - protein, 10 aa, synthetic construct
  REGION 1..10: note = Description of Artificial Sequence: Synthetic peptide
LRAHAVDVNG

SEQ ID NO: 342 - protein, 12 aa, synthetic construct
  REGION 1..12: note = Description of Artificial Sequence: Synthetic peptide
TAGSCLRKFSTM

SEQ ID NO: 343 - protein, 10 aa, synthetic construct
  REGION 1..10: note = Description of Artificial Sequence: Synthetic peptide
LFSHAVSSNG

SEQ ID NO: 344 - protein, 6 aa, synthetic construct
  REGION 1..6: note = Description of Artificial Sequence: Synthetic peptide
KGHRGF

SEQ ID NO: 345 - protein, 15 aa, synthetic construct
  REGION 1..15: note = Description of Artificial Sequence: Synthetic peptide
TAGSCLRKFSTMFLF

SEQ ID NO: 346 - protein, 18 aa, synthetic construct
  REGION 1..18: note = Description of Artificial Sequence: Synthetic peptide
TAGSCLRKFSTMFLFCNI

SEQ ID NO: 347 - protein, 15 aa, synthetic construct
  REGION 1..15: note = Description of Artificial Sequence: Synthetic peptide
DLGTAGSCLRKFSTM

SEQ ID NO: 348 - protein, 18 aa, synthetic construct
  REGION 1..18: note = Description of Artificial Sequence: Synthetic peptide
HQQDLGTAGSCLRKFSTM

SEQ ID NO: 349 - protein, 10 aa, synthetic construct
  REGION 1..10: note = Description of Artificial Sequence: Synthetic peptide
RNIAEIIKDI

SEQ ID NO: 350 - protein, 11 aa, synthetic construct
  REGION 1..11: note = Description of Artificial Sequence: Synthetic peptide
SIGFRGDGQTC

SEQ ID NO: 351 - protein, 10 aa, synthetic construct
  REGION 1..10: note = Description of Artificial Sequence: Synthetic peptide
LNRQELFPFG

SEQ ID NO: 352 - protein, 17 aa, synthetic construct
  REGION 1..17: note = Description of Artificial Sequence: Synthetic peptide
RIQNLLKITNLRIKFVK

SEQ ID NO: 353 - protein, 16 aa, synthetic construct
  REGION 1..16: note = Description of Artificial Sequence: Synthetic peptide
KKQRFRHRNRKGYRSQ

SEQ ID NO: 354 - protein, 12 aa, synthetic construct
  REGION 1..12: note = Description of Artificial Sequence: Synthetic peptide
SINNTAVMQRLT

SEQ ID NO: 355 - protein, 9 aa, synthetic construct
  REGION 1..9: note = Description of Artificial Sequence: Synthetic peptide
FRHRNRKGY

SEQ ID NO: 356 - protein, 17 aa, synthetic construct
  REGION 1..17: note = Description of Artificial Sequence: Synthetic peptide
RYRVRVTPKEKTGPMKE

SEQ ID NO: 357 - protein, 13 aa, synthetic construct
  REGION 1..13: note = Description of Artificial Sequence: Synthetic peptide
SETTVKYIFRLHE

SEQ ID NO: 358 - protein, 21 aa, synthetic construct
  REGION 1..21: note = Description of Artificial Sequence: Synthetic peptide
GHRGPTGRPGKRGKQGQKGDS

SEQ ID NO: 359 - protein, 12 aa, synthetic construct
  REGION 1..12: note = Description of Artificial Sequence: Synthetic peptide
KAFDITYVRLKF

SEQ ID NO: 360 - protein, 15 aa, synthetic construct
  REGION 1..15: note = Description of Artificial Sequence: Synthetic peptide
GDLGRPGRKGRPGPP

SEQ ID NO: 361 - protein, 5 aa, synthetic construct
  REGION 1..5: note = Description of Artificial Sequence: Synthetic peptide
YIGSR

SEQ ID NO: 362 - protein, 14 aa, synthetic construct
  REGION 1..14: note = Description of Artificial Sequence: Synthetic peptide
RGEFYFDLRLKGDK

SEQ ID NO: 363 - protein, 12 aa, synthetic construct
  REGION 1..12: note = Description of Artificial Sequence: Synthetic peptide
LAGSCLARFSTM

SEQ ID NO: 364 - protein, 12 aa, synthetic construct
  REGION 1..12: note = Description of Artificial Sequence: Synthetic peptide
LALFLSNGHFVA

SEQ ID NO: 365 - protein, 12 aa, synthetic construct
  REGION 1..12: note = Description of Artificial Sequence: Synthetic peptide
ISRCQVCMKKRH

SEQ ID NO: 366 - protein, 12 aa, synthetic construct
  REGION 1..12: note = Description of Artificial Sequence: Synthetic peptide
PGRWHKVSVRWE

SEQ ID NO: 367 - protein, 15 aa, synthetic construct
  REGION 1..15: note = Description of Artificial Sequence: Synthetic peptide
TDIPPCPHGWISLWK

SEQ ID NO: 368 - protein, 12 aa, synthetic construct
  REGION 1..12: note = Description of Artificial Sequence: Synthetic peptide
VRWGMQQIQLVV

SEQ ID NO: 369 - protein, 15 aa, synthetic construct
  REGION 1..15: note = Description of Artificial Sequence: Synthetic peptide
TAIPSCPEGTVPLYS

SEQ ID NO: 370 - protein, 12 aa, synthetic construct
  REGION 1..12: note = Description of Artificial Sequence: Synthetic peptide
KMPYVSLELEMR

SEQ ID NO: 371 - protein, 13 aa, synthetic construct
  REGION 1..13: note = Description of Artificial Sequence: Synthetic peptide
GPAGKDGEAGAQG

SEQ ID NO: 372 - protein, 12 aa, synthetic construct
  REGION 1..12: note = Description of Artificial Sequence: Synthetic peptide
VLLQANDGAGEF

SEQ ID NO: 373 - protein, 6 aa, synthetic construct
  REGION 1..6: note = Description of Artificial Sequence: Synthetic peptide
GLPGER

SEQ ID NO: 374 - protein, 12 aa, synthetic construct
  REGION 1..12: note = Description of Artificial Sequence: Synthetic peptide
DGRWHRVAVIMG

SEQ ID NO: 375 - protein, 12 aa, synthetic construct
  REGION 1..12: note = Description of Artificial Sequence: Synthetic peptide
LAGSCLPVFSTL

SEQ ID NO: 376 - protein, 12 aa, synthetic construct
  REGION 1..12: note = Description of Artificial Sequence: Synthetic peptide
APVNVTASVQIQ

SEQ ID NO: 377 - protein, 12 aa, synthetic construct
  REGION 1..12: note = Description of Artificial Sequence: Synthetic peptide
TAGSCLRRFSTM

SEQ ID NO: 378 - protein, 12 aa, synthetic construct
  REGION 1..12: note = Description of Artificial Sequence: Synthetic peptide
KQGKALTQRHAK

SEQ ID NO: 379 - protein, 9 aa, synthetic construct
  REGION 1..9: note = Description of Artificial Sequence: Synthetic peptide
TAGSCLRKF

SEQ ID NO: 380 - protein, 7 aa, synthetic construct
  REGION 1..7: note = Description of Artificial Sequence: Synthetic peptide
RYVVLPR

SEQ ID NO: 381 - protein, 6 aa, synthetic construct
  REGION 1..6: note = Description of Artificial Sequence: Synthetic peptide
TAGSCL

SEQ ID NO: 382 - protein, 14 aa, synthetic construct
  REGION 1..14: note = Description of Artificial Sequence: Synthetic peptide
SPYTFIDSLVLMPY

SEQ ID NO: 383 - skipped

SEQ ID NO: 384 - protein, 5 aa, synthetic construct
  REGION 1..5: note = Description of Artificial Sequence: Synthetic peptide
PDSGR

SEQ ID NO: 385 - protein, 12 aa, synthetic construct
  REGION 1..12: note = Description of Artificial Sequence: Synthetic peptide
QQNLGSVNVSTG

SEQ ID NO: 386 - protein, 12 aa, synthetic construct
  REGION 1..12: note = Description of Artificial Sequence: Synthetic peptide
SRATAQKVSRRS

SEQ ID NO: 387 - protein, 8 aa, synthetic construct
  REGION 1..8: note = Description of Artificial Sequence: Synthetic peptide
DPGYIGSR

SEQ ID NO: 388 - protein, 12 aa, synthetic construct
  REGION 1..12: note = Description of Artificial Sequence: Synthetic peptide
GSLSSHLEFVGI

SEQ ID NO: 389 - protein, 12 aa, synthetic construct
  REGION 1..12: note = Description of Artificial Sequence: Synthetic peptide
VILQQSAADIAR

SEQ ID NO: 390 - protein, 12 aa, synthetic construct
  REGION 1..12: note = Description of Artificial Sequence: Synthetic peptide
RNRLHLSMLVRP

SEQ ID NO: 391 - protein, 12 aa, synthetic construct
  REGION 1..12: note = Description of Artificial Sequence: Synthetic peptide
KDISEKVAVYST

SEQ ID NO: 392 - protein, 13 aa, synthetic construct
  REGION 1..13: note = Description of Artificial Sequence: Synthetic peptide
APMSGRSPSLVLK

SEQ ID NO: 393 - protein, 6 aa, synthetic construct
  REGION 1..6: note = Description of Artificial Sequence: Synthetic peptide
LGTIPG

SEQ ID NO: 394 - protein, 12 aa, synthetic construct
  REGION 1..12: note = Description of Artificial Sequence: Synthetic peptide
AFGVLALWGTRV

SEQ ID NO: 395 - protein, 13 aa, synthetic construct
  REGION 1..13: note = Description of Artificial Sequence: Synthetic peptide
TDIRVTLNRLNTF

SEQ ID NO: 396 - protein, 12 aa, synthetic construct
  REGION 1..12: note = Description of Artificial Sequence: Synthetic peptide
IENVVTTFAPNR

SEQ ID NO: 397 - protein, 12 aa, synthetic construct
  REGION 1..12: note = Description of Artificial Sequence: Synthetic peptide
AFSTLEGRPSAY

SEQ ID NO: 398 - protein, 12 aa, synthetic construct
  REGION 1..12: note = Description of Artificial Sequence: Synthetic peptide
LEAEFHFTHLIM

SEQ ID NO: 399 - protein, 12 aa, synthetic construct
  REGION 1..12: note = Description of Artificial Sequence: Synthetic peptide
TSAEAYNLLLRT

SEQ ID NO: 400 - protein, 12 aa, synthetic construct
  REGION 1..12: note = Description of Artificial Sequence: Synthetic peptide
HLIMTFKTFRPA

SEQ ID NO: 401 - protein, 12 aa, synthetic construct
  REGION 1..12: note = Description of Artificial Sequence: Synthetic peptide
LNRRYEQARNIS

SEQ ID NO: 402 - protein, 12 aa, synthetic construct
  REGION 1..12: note = Description of Artificial Sequence: Synthetic peptide
KTWGVYRYFAYD

SEQ ID NO: 403 - protein, 12 aa, synthetic construct
  REGION 1..12: note = Description of Artificial Sequence: Synthetic peptide
SLLSQLNNLLDQ

SEQ ID NO: 404 - protein, 12 aa, synthetic construct
  REGION 1..12: note = Description of Artificial Sequence: Synthetic peptide
TNLRIKFVKLHT

SEQ ID NO: 405 - protein, 10 aa, synthetic construct
  REGION 1..10: note = Description of Artificial Sequence: Synthetic peptide
RDIAEIIKDI

SEQ ID NO: 406 - protein, 8 aa, synthetic construct
  REGION 1..8: note = Description of Artificial Sequence: Synthetic peptide
KRLVTGQR

SEQ ID NO: 407 - protein, 6 aa, synthetic construct
  REGION 1..6: note = Description of Artificial Sequence: Synthetic peptide
SHAVSS

SEQ ID NO: 408 - protein, 12 aa, synthetic construct
  REGION 1..12: note = Description of Artificial Sequence: Synthetic peptide
GPGVVVVERQYI

SEQ ID NO: 409 - protein, 6 aa, synthetic construct
  REGION 1..6: note = Description of Artificial Sequence: Synthetic peptide
ADTPPV

SEQ ID NO: 410 - protein, 13 aa, synthetic construct
  REGION 1..13: note = Description of Artificial Sequence: Synthetic peptide
NEPKVLKSYYYAI

SEQ ID NO: 411 - protein, 10 aa, synthetic construct
  REGION 1..10: note = Description of Artificial Sequence: Synthetic peptide
LRAHAVDING

SEQ ID NO: 412 - protein, 12 aa, synthetic construct
  REGION 1..12: note = Description of Artificial Sequence: Synthetic peptide
YYAISDFAVGGR

SEQ ID NO: 413 - protein, 12 aa, synthetic construct
  REGION 1..12: note = Description of Artificial Sequence: Synthetic peptide
DSITKYFQMSLE

SEQ ID NO: 414 - protein, 13 aa, synthetic construct
  REGION 1..13: note = Description of Artificial Sequence: Synthetic peptide
LPFFNDRPWRRAT

SEQ ID NO: 415 - protein, 10 aa, synthetic construct
  REGION 1..10: note = Description of Artificial Sequence: Synthetic peptide
YTALIIATDN

SEQ ID NO: 416 - protein, 14 aa, synthetic construct
  REGION 1..14: note = Description of Artificial Sequence: Synthetic peptide
FDPELYRSTGHGGH

SEQ ID NO: 417 - protein, 10 aa, synthetic construct
  REGION 1..10: note = Description of Artificial Sequence: Synthetic peptide
VITVKDINDN

SEQ ID NO: 418 - protein, 12 aa, synthetic construct
  REGION 1..12: note = Description of Artificial Sequence: Synthetic peptide
TNAVGYSVYDIS

SEQ ID NO: 419 - protein, 10 aa, synthetic construct
  REGION 1..10: note = Description of Artificial Sequence: Synthetic peptide
GLDRESYPYY

SEQ ID NO: 420 - protein, 13 aa, synthetic construct
  REGION 1..13: note = Description of Artificial Sequence: Synthetic peptide
APVKFLGNQVLSY

SEQ ID NO: 421 - protein, 10 aa, synthetic construct
  REGION 1..10: note = Description of Artificial Sequence: Synthetic peptide
MKVSATDADD

SEQ ID NO: 422 - protein, 12 aa, synthetic construct
  REGION 1..12: note = Description of Artificial Sequence: Synthetic peptide
SFSFRVDRRDTR

SEQ ID NO: 423 - protein, 12 aa, synthetic construct
  REGION 1..12: note = Description of Artificial Sequence: Synthetic peptide
PQVTRGDVFTMP

SEQ ID NO: 424 - protein, 17 aa, synthetic construct
  REGION 1..17: note = Description of Artificial Sequence: Synthetic peptide
TWSKVGGHLRPGIVQSG

SEQ ID NO: 425 - protein, 12 aa, synthetic construct
  REGION 1..12: note = Description of Artificial Sequence: Synthetic peptide
KEAEREVTDLLR

SEQ ID NO: 426 - protein, 4 aa, synthetic construct
  REGION 1..4: note = Description of Artificial Sequence: Synthetic peptide
RGDV

SEQ ID NO: 427 - protein, 12 aa, synthetic construct
  REGION 1..12: note = Description of Artificial Sequence: Synthetic peptide
AAEPLKNIGILF

SEQ ID NO: 428 - protein, 11 aa, synthetic construct
  REGION 1..11: note = Description of Artificial Sequence: Synthetic peptide
FALWDAIIGEL

SEQ ID NO: 429 - protein, 6 aa, synthetic construct
  REGION 1..6: note = Description of Artificial Sequence: Synthetic peptide
VGVAPG

SEQ ID NO: 430 - protein, 12 aa, synthetic construct
  REGION 1..12: note = Description of Artificial Sequence: Synthetic peptide
LWPLLAVLAAVA

SEQ ID NO: 431 - protein, 5 aa, synthetic construct
  REGION 1..5: note = Description of Artificial Sequence: Synthetic peptide
PGVGV

SEQ ID NO: 432 - protein, 8 aa, synthetic construct
  REGION 1..8: note = Description of Artificial Sequence: Synthetic peptide
VFDNFVLK

SEQ ID NO: 433 - protein, 12 aa, synthetic construct
  REGION 1..12: note = Description of Artificial Sequence: Synthetic peptide
TSIKIRGTYSER

SEQ ID NO: 434 - protein, 10 aa, synthetic construct
  REGION 1..10: note = Description of Artificial Sequence: Synthetic peptide
TTSWSQCSKS

SEQ ID NO: 435 - protein, 6 aa, synthetic construct
  REGION 1..6: note = Description of Artificial Sequence: Synthetic peptide
DPETGV

SEQ ID NO: 436 - protein, 4 aa, synthetic construct
  REGION 1..4: note = Description of Artificial Sequence: Synthetic peptide
KRSR

SEQ ID NO: 437 - protein, 10 aa, synthetic construct
  REGION 1..10: note = Description of Artificial Sequence: Synthetic peptide
QGADTPPVGV

SEQ ID NO: 438 - protein, 7 aa, synthetic construct
  REGION 1..7: note = Description of Artificial Sequence: Synthetic peptide
SVVYGLR

SEQ ID NO: 439 - protein, 10 aa, synthetic construct
  REGION 1..10: note = Description of Artificial Sequence: Synthetic peptide
PLDREAIAKY

SEQ ID NO: 440 - protein, 10 aa, synthetic construct
  REGION 1..10: note = Description of Artificial Sequence: Synthetic peptide
DGRGDSVAYG

SEQ ID NO: 441 - protein, 5 aa, synthetic construct
  REGION 1..5: note = Description of Artificial Sequence: Synthetic peptide
HAVDI

SEQ ID NO: 442 - protein, 10 aa, synthetic construct
  REGION 1..10: note = Description of Artificial Sequence: Synthetic peptide
LALERKDHSG

SEQ ID NO: 443 - protein, 5 aa, synthetic construct
  REGION 1..5: note = Description of Artificial Sequence: Synthetic peptide
DQNDN

SEQ ID NO: 444 - protein, 19 aa, synthetic construct
  REGION 1..19: note = Description of Artificial Sequence: Synthetic peptide
YSMKKTTMKIIPFNRLTIG

SEQ ID NO: 445 - protein, 10 aa, synthetic construct
  REGION 1..10: note = Description of Artificial Sequence: Synthetic peptide
QDPELPDKNM

SEQ ID NO: 446 - protein, 4 aa, synthetic construct
  REGION 1..4: note = Description of Artificial Sequence: Synthetic peptide
RGDF

SEQ ID NO: 447 - protein, 10 aa, synthetic construct
  REGION 1..10: note = Description of Artificial Sequence: Synthetic peptide
LVVQAADLQG

SEQ ID NO: 448 - protein, 13 aa, synthetic construct
  REGION 1..13: note = Description of Artificial Sequence: Synthetic peptide
GVYYQGGTYSKAS

SEQ ID NO: 449 - protein, 10 aa, synthetic construct
  REGION 1..10: note = Description of Artificial Sequence: Synthetic peptide
NDDGGQFVVT

SEQ ID NO: 450 - protein, 12 aa, synthetic construct
  REGION 1..12: note = Description of Artificial Sequence: Synthetic peptide
TAGSCLRKFSCL

SEQ ID NO: 451 - protein, 9 aa, synthetic construct
  REGION 1..9: note = Description of Artificial Sequence: Synthetic peptide
YILHVAVTN

SEQ ID NO: 452 - protein, 19 aa, synthetic construct
  REGION 1..19: note = Description of Artificial Sequence: Synthetic peptide
CNYYSNSYSFWLASLNPER

SEQ ID NO: 453 - protein, 10 aa, synthetic construct
  REGION 1..10: note = Description of Artificial Sequence: Synthetic peptide
TYRIWRDTAN

SEQ ID NO: 454 - protein, 12 aa, synthetic construct
  REGION 1..12: note = Description of Artificial Sequence: Synthetic peptide
TGLSCLQRFTTM

SEQ ID NO: 455 - protein, 26 aa, synthetic construct
  REGION 1..26: note = Description of Artificial Sequence: Synthetic peptide
GFTCECSIGFRGDGQTCYGIVFWSEV

SEQ ID NO: 456 - protein, 12 aa, synthetic construct
  REGION 1..12: note = Description of Artificial Sequence: Synthetic peptide
HHLGGAKQAGDV

SEQ ID NO: 457 - protein, 21 aa, synthetic construct
  REGION 1..21: note = Description of Artificial Sequence: Synthetic peptide
SCLPGFSGDGRACRDVDECGH

SEQ ID NO: 458 - protein, 30 aa, synthetic construct
  REGION 1..30: note = Description of Artificial Sequence: Synthetic polypeptide
MAPRPSLAKKQRFRHRNRKGYRSQRGHSRG

SEQ ID NO: 459 - protein, 16 aa, synthetic construct
  REGION 1..16: note = Description of Artificial Sequence: Synthetic peptide
KKQKFRHRNRKGYRSQ

SEQ ID NO: 460 - protein, 15 aa, synthetic construct
  REGION 1..15: note = Description of Artificial Sequence: Synthetic peptide
KKQKFKHRNRKGYRS

SEQ ID NO: 461 - protein, 16 aa, synthetic construct
  REGION 1..16: note = Description of Artificial Sequence: Synthetic peptide
KKQKFRRRNRKGYRSH

SEQ ID NO: 462 - protein, 15 aa, synthetic construct
  REGION 1..15: note = Description of Artificial Sequence: Synthetic peptide
TAIPPCPHGWISLWK

SEQ ID NO: 463 - protein, 15 aa, synthetic construct
  REGION 1..15: note = Description of Artificial Sequence: Synthetic peptide
KKQKSRHRSRKRYRS

SEQ ID NO: 464 - protein, 15 aa, synthetic construct
  REGION 1..15: note = Description of Artificial Sequence: Synthetic peptide
KKQKSRRRSRKGYRS

SEQ ID NO: 465 - protein, 7 aa, synthetic construct
  REGION 1..7: note = Description of Artificial Sequence: Synthetic peptide
ISRCTVC

SEQ ID NO: 466 - protein, 12 aa, synthetic construct
  REGION 1..12: note = Description of Artificial Sequence: Synthetic peptide
ISRCQVCMKRRH

SEQ ID NO: 467 - protein, 7 aa, synthetic construct
  REGION 1..7: note = Description of Artificial Sequence: Synthetic peptide
VSRCTVC

SEQ ID NO: 468 - protein, 15 aa, synthetic construct
  REGION 1..15: note = Description of Artificial Sequence: Synthetic peptide
TDIPPCPQGWISLWK

SEQ ID NO: 469 - protein, 21 aa, synthetic construct
  REGION 1..21: note = Description of Artificial Sequence: Synthetic peptide
TVKAGELEKIISRCQVMKKRH

SEQ ID NO: 470 - protein, 15 aa, synthetic construct
  REGION 1..15: note = Description of Artificial Sequence: Synthetic peptide
TDIPSCPHGWISLWK

SEQ ID NO: 471 - protein, 15 aa, synthetic construct
  REGION 1..15: note = Description of Artificial Sequence: Synthetic peptide
TDIPPCPAGWISLWK

SEQ ID NO: 472 - protein, 15 aa, synthetic construct
  REGION 1..15: note = Description of Artificial Sequence: Synthetic peptide
TEIPPCPQGWISLWK

SEQ ID NO: 473 - protein, 15 aa, synthetic construct
  REGION 1..15: note = Description of Artificial Sequence: Synthetic peptide
TDVPPCPQGWISLWK

SEQ ID NO: 474 - protein, 13 aa, synthetic construct
  REGION 1..13: note = Description of Artificial Sequence: Synthetic peptide
RLVSYNGILFFLK

SEQ ID NO: 475 - protein, 13 aa, synthetic construct
  REGION 1..13: note = Description of Artificial Sequence: Synthetic peptide
RLVSYSGVIFFLK

SEQ ID NO: 476 - protein, 12 aa, synthetic construct
  REGION 1..12: note = Description of Artificial Sequence: Synthetic peptide
RLVSYNGILFFL

SEQ ID NO: 477 - protein, 13 aa, synthetic construct
  REGION 1..13: note = Description of Artificial Sequence: Synthetic peptide
RLVSYSGIIFFLK

SEQ ID NO: 478 - protein, 20 aa, synthetic construct
  REGION 1..20: note = Description of Artificial Sequence: Synthetic peptide
RFEQELRLVSYSGVLFFLKQ

SEQ ID NO: 479 - protein, 13 aa, synthetic construct
  REGION 1..13: note = Description of Artificial Sequence: Synthetic peptide
RLVSYNGIIFFLK

SEQ ID NO: 480 - protein, 30 aa, synthetic construct
  REGION 1..30: note = Description of Artificial Sequence: Synthetic polypeptide
DPAFKIEDPYSPRIQNLLKITNLRIKFVKL

SEQ ID NO: 481 - protein, 20 aa, synthetic construct
  REGION 1..20: note = Description of Artificial Sequence: Synthetic peptide
TKRFEQELRLVSYSGVLFFL

SEQ ID NO: 482 - protein, 10 aa, synthetic construct
  REGION 1..10: note = Description of Artificial Sequence: Synthetic peptide
GGRLKYSVAF

SEQ ID NO: 483 - protein, 12 aa, synthetic construct
  REGION 1..12: note = Description of Artificial Sequence: Synthetic peptide
GGFLRYTVSYDI

SEQ ID NO: 484 - protein, 12 aa, synthetic construct
  REGION 1..12: note = Description of Artificial Sequence: Synthetic peptide
GGFLKYTVSYDV

SEQ ID NO: 485 - protein, 22 aa, synthetic construct
  REGION 1..22: note = Description of Artificial Sequence: Synthetic peptide
LGNKLTAFGGFLKYTVSYDIPV

SEQ ID NO: 486 - protein, 12 aa, synthetic construct
  REGION 1..12: note = Description of Artificial Sequence: Synthetic peptide
GGYLKYTVSYDI

SEQ ID NO: 487 - protein, 13 aa, synthetic construct
  REGION 1..13: note = Description of Artificial Sequence: Synthetic peptide
GEIFFDMRLKGDK

SEQ ID NO: 488 - protein, 13 aa, synthetic construct
  REGION 1..13: note = Description of Artificial Sequence: Synthetic peptide
GEIYFDLRLKGDK

SEQ ID NO: 489 - protein, 13 aa, synthetic construct
  REGION 1..13: note = Description of Artificial Sequence: Synthetic peptide
GEIYLDMRLKGDK

SEQ ID NO: 490 - protein, 30 aa, synthetic construct
  REGION 1..30: note = Description of Artificial Sequence: Synthetic polypeptide
IGQPGAKGEPGEFYFDLRLKGDKGDPGFPG

SEQ ID NO: 491 - protein, 13 aa, synthetic construct
  REGION 1..13: note = Description of Artificial Sequence: Synthetic peptide
GEVFFDMRLKGDK

SEQ ID NO: 492 - protein, 12 aa, synthetic construct
  REGION 1..12: note = Description of Artificial Sequence: Synthetic peptide
LAGSCLPIFSTL

SEQ ID NO: 493 - protein, 30 aa, synthetic construct
  REGION 1..30: note = Description of Artificial Sequence: Synthetic polypeptide
AHNQDLGLAGSCLARFSTMPFLYCNPGDIC

SEQ ID NO: 494 - protein, 30 aa, synthetic construct
  REGION 1..30: note = Description of Artificial Sequence: Synthetic polypeptide
QEKAHNQDLGLAGSCLPVFSTLPFAYCNIH

SEQ ID NO: 495 - protein, 12 aa, synthetic construct
  REGION 1..12: note = Description of Artificial Sequence: Synthetic peptide
LAGSCLPVFSTM

SEQ ID NO: 496 - protein, 30 aa, synthetic construct
  REGION 1..30: note = Description of Artificial Sequence: Synthetic polypeptide
GNKRAHGQDLGTAGSCLRRFSTMPFMFCNI

SEQ ID NO: 497 - protein, 21 aa, synthetic construct
  REGION 1..21: note = Description of Artificial Sequence: Synthetic peptide
RAHGQDLGTAGSCLRRFSTMP

SEQ ID NO: 498 - protein, 12 aa, synthetic construct
  REGION 1..12: note = Description of Artificial Sequence: Synthetic peptide
RKRLQVQLNIRT

SEQ ID NO: 499 - protein, 30 aa, synthetic construct
  REGION 1..30: note = Description of Artificial Sequence: Synthetic polypeptide
HLVLPLQQSDVRKRLQVQLSIRTFASSGLI

SEQ ID NO: 500 - protein, 12 aa, synthetic construct
  REGION 1..12: note = Description of Artificial Sequence: Synthetic peptide
RKRLSVQLRIRT

SEQ ID NO: 501 - protein, 15 aa, synthetic construct
  REGION 1..15: note = Description of Artificial Sequence: Synthetic peptide
DLGTAGSCLRRFSTM

SEQ ID NO: 502 - protein, 10 aa, synthetic construct
  REGION 1..10: note = Description of Artificial Sequence: Synthetic peptide
RNIAEIIKDI

SEQ ID NO: 503 - protein, 24 aa, synthetic construct
  REGION 1..24: note = Description of Artificial Sequence: Synthetic peptide
TAGSCLRKFSTMRRRRRRRRRRRR

SEQ ID NO: 504 - protein, 17 aa, synthetic construct
  REGION 1..17: note = Description of Artificial Sequence: Synthetic peptide
FTLTGLLGTLVTMGLLT

SEQ ID NO: 505 - protein, 7 aa, synthetic construct
  REGION 1..7: note = Description of Artificial Sequence: Synthetic peptide
APYKAWK

SEQ ID NO: 506 - protein, 23 aa, synthetic construct
  REGION 1..23: note = Description of Artificial Sequence: Synthetic peptide
STSKTNRGDDSNWSKRVTNNKPS

SEQ ID NO: 507 - protein, 23 aa, synthetic construct
  REGION 1..23: note = Description of Artificial Sequence: Synthetic peptide
STSKRKRGDDSNWSKRVTKKKPS

SEQ ID NO: 508 - protein, 23 aa, synthetic construct
  REGION 1..23: note = Description of Artificial Sequence: Synthetic peptide
STSKRKRGDDSNWSKRVSKKKPS

SEQ ID NO: 509 - protein, 23 aa, synthetic construct
  REGION 1..23: note = Description of Artificial Sequence: Synthetic peptide
STSKRKRGDDANWSKRVTKKKPS

SEQ ID NO: 510 - protein, 26 aa, synthetic construct
  REGION 1..26: note = Description of Artificial Sequence: Synthetic peptide
PLAGSKRKRADEVAWSKRGTKKKPER

SEQ ID NO: 511 - protein, 39 aa, synthetic construct
  REGION 1..39: note = Description of Artificial Sequence: Synthetic polypeptide
PLAGSKRKRADEVAWSKRGTKKKPERTSAARAGPSRRIR

SEQ ID NO: 512 - protein, 24 aa, synthetic construct
  REGION 1..24: note = Description of Artificial Sequence: Synthetic peptide
STSKRKRGDDANWSKRTTKKKPSS

SEQ ID NO: 513 - protein, 39 aa, synthetic construct
  REGION 1..39: note = Description of Artificial Sequence: Synthetic polypeptide
STSKRKRGDDANWSKRTTKKKPSSAGLKRAGSKADRPSL

SEQ ID NO: 514 - protein, 26 aa, synthetic construct
  REGION 1..26: note = Description of Artificial Sequence: Synthetic peptide
PTTAGKRKRSDDAAWSKRARPKAGRT

SEQ ID NO: 515 - protein, 39 aa, synthetic construct
  REGION 1..39: note = Description of Artificial Sequence: Synthetic polypeptide
PTTAGKRKRSDDAAWSKRARPKAGRTSAARPGTSVRRIR

SEQ ID NO: 516 - protein, 26 aa, synthetic construct
  REGION 1..26: note = Description of Artificial Sequence: Synthetic peptide
SSSLGKRKRSDEGAWSKGKSKKKAMR

SEQ ID NO: 517 - protein, 39 aa, synthetic construct
  REGION 1..39: note = Description of Artificial Sequence: Synthetic polypeptide
SSSLGKRKRSDEGAWSKGKSKKKAMRGSSSRRPGPVRGP

SEQ ID NO: 518 - protein, 25 aa, synthetic construct
  REGION 1..25: note = Description of Artificial Sequence: Synthetic peptide
PTTAGKRKRTDDAAWSKRARPKAGR

SEQ ID NO: 519 - protein, 39 aa, synthetic construct
  REGION 1..39: note = Description of Artificial Sequence: Synthetic polypeptide
PTTAGKRKRTDDAAWSKRARPKAGRTSAARPGTAVRRVR

SEQ ID NO: 520 - protein, 30 aa, synthetic construct
  REGION 1..30: note = Description of Artificial Sequence: Synthetic polypeptide
PATAGKRKRSDDAAWSKRARPKAGRTSAAR

SEQ ID NO: 521 - protein, 39 aa, synthetic construct
  REGION 1..39: note = Description of Artificial Sequence: Synthetic polypeptide
PATAGKRKRSDDAAWSKRARPKAGRTSAARPGTSVRRIR

SEQ ID NO: 522 - protein, 25 aa, synthetic construct
  REGION 1..25: note = Description of Artificial Sequence: Synthetic peptide
SSSLGKRKRSNGGDWSKRSAKKKPA

SEQ ID NO: 523 - protein, 39 aa, synthetic construct
  REGION 1..39: note = Description of Artificial Sequence: Synthetic polypeptide
SSSLGKRKRSNGGDWSKRSAKKKPAGTPSRRAGPGRGPR

SEQ ID NO: 524 - protein, 26 aa, synthetic construct
  REGION 1..26: note = Description of Artificial Sequence: Synthetic peptide
SSSLGKRKRSDEGAWSKGKSKKKAMR

SEQ ID NO: 525 - protein, 39 aa, synthetic construct; identical to SEQ ID NO: 517
  REGION 1..39: note = Description of Artificial Sequence: Synthetic polypeptide

SEQ ID NO: 526 - protein, 24 aa, synthetic construct
  REGION 1..24: note = Description of Artificial Sequence: Synthetic peptide
STSKRKRGDDANWNKRPTKKKPSS

SEQ ID NO: 527 - protein, 39 aa, synthetic construct
  REGION 1..39: note = Description of Artificial Sequence: Synthetic polypeptide
STSKRKRGDDANWNKRPTKKKPSSAGLKKAGSKAERPSL

SEQ ID NO: 528 - protein, 24 aa, synthetic construct
  REGION 1..24: note = Description of Artificial Sequence: Synthetic peptide
SGALKRKRSDEVAWSRRRPVKKPV

SEQ ID NO: 529 - protein, 39 aa, synthetic construct
  REGION 1..39: note = Description of Artificial Sequence: Synthetic polypeptide
SGALKRKRSDEVAWSRRRPVKKPVRRAPPPRAGPSVRRG

SEQ ID NO: 530 - protein, 25 aa, synthetic construct
  REGION 1..25: note = Description of Artificial Sequence: Synthetic peptide
SGALKRKRSDEVAWSRRKPAKKPAR

SEQ ID NO: 531 - protein, 39 aa, synthetic construct
  REGION 1..39: note = Description of Artificial Sequence: Synthetic polypeptide
SGALKRKRSDEVAWSRRKPAKKPARQPPPPRAGPSVRRG

SEQ ID NO: 532 - protein, 25 aa, synthetic construct
  REGION 1..25: note = Description of Artificial Sequence: Synthetic peptide
AGALKRKRSDEVAWSRRKPAKKPAR

SEQ ID NO: 533 - protein, 39 aa, synthetic construct
  REGION 1..39: note = Description of Artificial Sequence: Synthetic polypeptide
AGALKRKRSDEVAWSRRKPAKKPARAPPPRAGPSVRRGL

SEQ ID NO: 534 - protein, 243 aa, synthetic construct
  REGION 1..243: note = Description of Artificial Sequence: Synthetic polypeptide
STSKRKRGDDSNWSKRVTKKKPSSAGLKRAGSKADRPSLQIQTLQHAGTTMITVPSGGVCDLINTYARGSDEGNRHTSETLTYKIAIDYHFVADAAACRYSNTGTGVMWLVYDTTPGGQAPTPQTIFSYPDTLKAWPATWKVSRELCHRFVVKRRWLFNMETDGRIGSDIPPSNASWKPCKRNIYFHKFTSGLGVRTQWKNVTDGGVGAIQRGALYMVIAPGNGLTFTAHGQTRLYFKSVGNQ

SEQ ID NO: 535 - protein, 100 aa, synthetic construct
  REGION 1..100: note = Description of Artificial Sequence: Synthetic polypeptide
DPQNALYYQPRVPTAAPTSGGVPWSRVGEVAILSFVALICFYLLYLWVLRDLILVLKARQGRSTEELIFGGQAVDRSNPIPNIPAPPSQGNPGPFVPGTG

SEQ ID NO: 536 - protein, 292 aa, synthetic construct
  REGION 1..292: note = Description of Artificial Sequence: Synthetic polypeptide
GSQLVPPPSAFNYIESQRDEFQLSHDLTEIVLQFPSTASQITARLSRSCMKIDHCVIEYRQQVPINASGTVIVEIHDKRMTDNESLQASWTFPIRCNIDLHYFSSSFFSLKDPIPWKLYYRVSDSNVHQMTHFAKFKGKLKLSSAKHSVDIPFRAPTVKILAKQFSEKDIDFWHVGYGKWERRLVKSASSSRFGLRGPIEINPGESWATKSAIVTPNRNADLDIEEELLPYRELNRLGTNILDPGESASIVGIQRSQSNITMSMSQLNELVRSTVHECIKTSCIPSTPKSLS

SEQ ID NO: 537 - protein, 22 aa, synthetic construct
  REGION 1..22: note = Description of Artificial Sequence: Synthetic peptide
RTGVKRSYGAARGDDRRRPNVV

SEQ ID NO: 538 - protein, 19 aa, synthetic construct
  REGION 1..19: note = Description of Artificial Sequence: Synthetic peptide
SYVKTVPNRTRTYIKLRVR

SEQ ID NO: 539 - protein, 255 aa, synthetic construct
  REGION 1..255: note = Description of Artificial Sequence: Synthetic polypeptide
MYSTSNRRGRSQTQRGSHVRRTGVKRSYGAARGDDRRRPNVVSKTQVEPRMTIQRVQENQFGPEFVLSQNSALSTFVTYPSYVKTVPNRTRTYIKLKRVRFKGTLKIERGQGDTIMDGPSSNIEGVFSMVIVVDRKPHVSQSGRLHTFDELFGARIHCHGNLSVVPALKDRYYIRHVTKRVVSLEKDTLLIDLHGTTQLSNKRYNCWASFSDLERDCNGVYGNITKNALLVYYCWLSDAQSKASTYVSFELDYLG

SEQ ID NO: 540 - protein, 30 aa, synthetic construct
  REGION 1..30: note = Description of Artificial Sequence: Synthetic polypeptide
RRRRRRRRRRRRVDYGKWERKPIRCASMSR

SEQ ID NO: 541 - protein, 24 aa, synthetic construct
  REGION 1..24: note = Description of Artificial Sequence: Synthetic peptide
RRRRRRRRRRRRGKWERKPIRCAS

SEQ ID NO: 542 - protein, 28 aa, synthetic construct
  REGION 1..28: note = Description of Artificial Sequence: Synthetic peptide
KKKKKKKKKKKKKKKKGKWERKPIRCAS

SEQ ID NO: 543 - protein, 40 aa, synthetic construct
  REGION 1..40: note = Description of Artificial Sequence: Synthetic polypeptide
RRRRRRRRRRRRVDFSHVDYGKWERKPIRCASMSRLGLRG

SEQ ID NO: 544 - protein, 38 aa, synthetic construct
  REGION 1..38: note = Description of Artificial Sequence: Synthetic polypeptide
GVKRSYGAARGDDRRRPNVVAPYKAWRRRRRRRRRRRR

SEQ ID NO: 545 - protein, 38 aa, synthetic construct
  REGION 1..38: note = Description of Artificial Sequence: Synthetic polypeptide
KSVPNRTRTYIKLKRLRFKGAPYKAWRRRRRRRRRRRR

SEQ ID NO: 546 - protein, 34 aa, synthetic construct
  REGION 1..34: note = Description of Artificial Sequence: Synthetic polypeptide
RTGVKRSYGAARGDDRRRPNVVRRRRRRRRRRRR

SEQ ID NO: 547 - protein, 32 aa, synthetic construct
  REGION 1..32: note = Description of Artificial Sequence: Synthetic polypeptide
SYVKTVPNRTRTYIKGGGGGRRRRRRRRRRRR

SEQ ID NO: 548 - protein, 50 aa, synthetic construct
  REGION 1..50: note = Description of Artificial Sequence: Synthetic polypeptide
VDIPFRAPTIKILSKQFTEDDIDFWHVGYGKWERKLVRPASLSGRRGLRR

SEQ ID NO: 549 - protein, 29 aa, synthetic construct
  REGION 1..29: note = Description of Artificial Sequence: Synthetic peptide
IDFWHVGYGKWERKLVRPASLSGRRGLRR

SEQ ID NO: 550 - protein, 31 aa, synthetic construct
  REGION 1..31: note = Description of Artificial Sequence: Synthetic polypeptide
IDFWSVEKGETRRRLLNPTPHAHSPRPIAHR

SEQ ID NO: 551 - protein, 28 aa, synthetic construct
  REGION 1..28: note = Description of Artificial Sequence: Synthetic peptide
IDFSHVGYGKWERKMIRSASISRLGLHN

SEQ ID NO: 552 - protein, 28 aa, synthetic construct
  REGION 1..28: note = Description of Artificial Sequence: Synthetic peptide
VDFSHVGYGKWERKLIRSASTVKYGLPS

SEQ ID NO: 553 - protein, 28 aa, synthetic construct
  REGION 1..28: note = Description of Artificial Sequence: Synthetic peptide
IDFSHVDYGKVERKLVKCESSSRLGLHS

SEQ ID NO: 554 - protein, 29 aa, synthetic construct
  REGION 1..29: note = Description of Artificial Sequence: Synthetic peptide
IDFWSVGRKAQQRKLVQGPSLIGSRSMRY

SEQ ID NO: 555 - protein, 31 aa, synthetic construct
  REGION 1..31: note = Description of Artificial Sequence: Synthetic polypeptide
IDFWSVGSKPQTRRLVDGSRLIGHSSRSLRV

SEQ ID NO: 556 - protein, 31 aa, synthetic construct
  REGION 1..31: note = Description of Artificial Sequence: Synthetic polypeptide
IDFWSVERGETRRRLLNPTPSAGSNRALSKR

SEQ ID NO: 557 - protein, 31 aa, synthetic construct
  REGION 1..31: note = Description of Artificial Sequence: Synthetic polypeptide
VDFWSVGKPKPIRRLIQNDPGTDYDTGPKYR

SEQ ID NO: 558 - protein, 31 aa, synthetic construct
  REGION 1..31: note = Description of Artificial Sequence: Synthetic polypeptide
VDFWSVEKPKPIRRLLNPGPNQGPYPNTGHR

SEQ ID NO: 559 - protein, 28 aa, synthetic construct
  REGION 1..28: note = Description of Artificial Sequence: Synthetic peptide
VDFSHVDYGKWERKLIRSASTSRYGLRS

SEQ ID NO: 560 - protein, 28 aa, synthetic construct
  REGION 1..28: note = Description of Artificial Sequence: Synthetic peptide
VDFSHVDYGKWERKTLRSRSLSRIGLTG

SEQ ID NO: 561 - protein, 28 aa, synthetic construct
  REGION 1..28: note = Description of Artificial Sequence: Synthetic peptide
IDFWHVGYGKWERRLVKSASSSRFGIRG

SEQ ID NO: 562 - protein, 28 aa, synthetic construct
  REGION 1..28: note = Description of Artificial Sequence: Synthetic peptide
VDFFHVDYGRWERKHIRCASMSRVGLRG

SEQ ID NO: 563 - protein, 28 aa, synthetic construct
  REGION 1..28: note = Description of Artificial Sequence: Synthetic peptide
GTFQHVDYGKWERKPIRCQSMSRVGYRR

SEQ ID NO: 564 - protein, 17 aa, synthetic construct
  REGION 1..17: note = Description of Artificial Sequence: Synthetic peptide
VGYGKWERKLVRPASLS

SEQ ID NO: 565 - protein, 17 aa, synthetic construct
  REGION 1..17: note = Description of Artificial Sequence: Synthetic peptide
VEKGETRRRLLNPTPHA

SEQ ID NO: 566 - protein, 17 aa, synthetic construct
  REGION 1..17: note = Description of Artificial Sequence: Synthetic peptide
VGYGKWERKLIRSASTV

SEQ ID NO: 567 - protein, 17 aa, synthetic construct
  REGION 1..17: note = Description of Artificial Sequence: Synthetic peptide
VEKPKPIRRLLNPGPNQ

SEQ ID NO: 568 - protein, 17 aa, synthetic construct
  REGION 1..17: note = Description of Artificial Sequence: Synthetic peptide
VDYGKWERKLIRSASTS

SEQ ID NO: 569 - protein, 17 aa, synthetic construct
  REGION 1..17: note = Description of Artificial Sequence: Synthetic peptide
VDYGKWERKTLRSRSLS

SEQ ID NO: 570 - protein, 17 aa, synthetic construct
  REGION 1..17: note = Description of Artificial Sequence: Synthetic peptide
VGYGKWERRLVKSASSS

SEQ ID NO: 571 - protein, 17 aa, synthetic construct
  REGION 1..17: note = Description of Artificial Sequence: Synthetic peptide
VDYGRWERKHIRCASMS

SEQ ID NO: 572 - protein, 17 aa, synthetic construct
  REGION 1..17: note = Description of Artificial Sequence: Synthetic peptide
VERPKPIRRLLTPTPGC

SEQ ID NO: 573 - protein, 47 aa, synthetic construct
  REGION 1..47: note = Description of Artificial Sequence: Synthetic polypeptide
PFRAPTIKILSKQFTEDDIDFWHVGYGKWERKLVRPASLSGRRGLRR

SEQ ID NO: 574 - protein, 44 aa, synthetic construct
  REGION 1..44: note = Description of Artificial Sequence: Synthetic polypeptide
PFRAPTVKILSKQFTDKDIDFSHVGYGKWERKMIRSASISRLGL

SEQ ID NO: 575 - protein, 46 aa, synthetic construct
  REGION 1..46: note = Description of Artificial Sequence: Synthetic polypeptide
DIAFRAPTVKILSKQFTDRDVDFSHVGYGKWERKLIRSASTVKYGL

SEQ ID NO: 576 - protein, 49 aa, synthetic construct
  REGION 1..49: note = Description of Artificial Sequence: Synthetic polypeptide
DIRFKPPTINILSKDYTADCVDFWSVEKPKPIRRLLNPGPNQGPYPNTG

SEQ ID NO: 577 - protein, 46 aa, synthetic construct
  REGION 1..46: note = Description of Artificial Sequence: Synthetic polypeptide
DIPFRAPTVKIHSKQFSHRDVDFSHVDYGKWERKTLRSRSLSRIGL

SEQ ID NO: 578 - protein, 46 aa, synthetic construct
  REGION 1..46: note = Description of Artificial Sequence: Synthetic polypeptide
DIPFRAPTVKILAKQFSEKDIDFWHVGYGKWERRLVKSASSSRFGI

SEQ ID NO: 579 - protein, 46 aa, synthetic construct
  REGION 1..46: note = Description of Artificial Sequence: Synthetic polypeptide
DIPFRAPTVKILSKQFTDKDVDFFHVDYGRWERKHIRCASMSRVGL

SEQ ID NO: 580 - protein, 43 aa, synthetic construct
  REGION 1..43: note = Description of Artificial Sequence: Synthetic polypeptide
DIKYKPPTIKILSKDYTADCVDFWSVERPKPIRRLLTPTPGCG

SEQ ID NO: 581 - protein, 47 aa, synthetic construct
  REGION 1..47: note = Description of Artificial Sequence: Synthetic polypeptide
ARTKQTARKSTGGKAPRKQLATKAARKSAPATGGVKKPHRYRPGTVA

SEQ ID NO: 582 - protein, 34 aa, synthetic construct
  REGION 1..34: note = Description of Artificial Sequence: Synthetic polypeptide
SGRGKGGKGLGKGGAKRHRKVLRDNIQGITKPAI

SEQ ID NO: 583 - protein, 20 aa, synthetic construct
  REGION 1..20: note = Description of Artificial Sequence: Synthetic peptide
  REGION 1..20: note = MISC_FEATURE - This sequence may encompass 8 to 20 residues
KKKKKKKKKKKKKKKKKKKK

SEQ ID NO: 584 - protein, 20 aa, synthetic construct
  REGION 1..20: note = Description of Artificial Sequence: Synthetic peptide
  REGION 1..20: note = MISC_FEATURE - This sequence may encompass 8 to 20 residues
RRRRRRRRRRRRRRRRRRRR

SEQ ID NO: 585 - protein, 240 aa, synthetic construct
  REGION 1..240: note = Description of Artificial Sequence: Synthetic polypeptide
  REGION 1..6: note = MISC_FEATURE - This region may encompass 1-6 residues
  REGION 7..12: note = MISC_FEATURE - This region may encompass 1-6 residues
  REGION 13..18: note = MISC_FEATURE - This region may encompass 1-6 residues
  REGION 19..24: note = MISC_FEATURE - This region may encompass 1-6 residues
  REGION 25..30: note = MISC_FEATURE - This region may encompass 1-6 residues
  REGION 31..36: note = MISC_FEATURE - This region may encompass 1-6 residues
  REGION 37..42: note = MISC_FEATURE - This region may encompass 1-6 residues
  REGION 43..48: note = MISC_FEATURE - This region may encompass 1-6 residues
  REGION 49..54: note = MISC_FEATURE - This region may encompass 1-6 residues
  REGION 55..60: note = MISC_FEATURE - This region may encompass 1-6 residues
  REGION 61..66: note = MISC_FEATURE - This region may encompass 1-6 residues
  REGION 67..72: note = MISC_FEATURE - This region may encompass 1-6 residues
  REGION 73..78: note = MISC_FEATURE - This region may encompass 1-6 residues
  REGION 79..84: note = MISC_FEATURE - This region may encompass 1-6 residues
  REGION 85..90: note = MISC_FEATURE - This region may encompass 1-6 residues
  REGION 91..96: note = MISC_FEATURE - This region may encompass 1-6 residues
  REGION 97..102: note = MISC_FEATURE - This region may encompass 1-6 residues
  REGION 103..108: note = MISC_FEATURE - This region may encompass 1-6 residues
  REGION 109..114: note = MISC_FEATURE - This region may encompass 1-6 residues
  REGION 115..120: note = MISC_FEATURE - This region may encompass 1-6 residues
  REGION 121..126: note = MISC_FEATURE - This region may encompass 1-6 residues
  REGION 127..132: note = MISC_FEATURE - This region may encompass 1-6 residues
  REGION 133..138: note = MISC_FEATURE - This region may encompass 1-6 residues
  REGION 139..144: note = MISC_FEATURE - This region may encompass 1-6 residues
  REGION 145..150: note = MISC_FEATURE - This region may encompass 1-6 residues
  REGION 151..156: note = MISC_FEATURE - This region may encompass 1-6 residues
  REGION 157..162: note = MISC_FEATURE - This region may encompass 1-6 residues
  REGION 163..168: note = MISC_FEATURE - This region may encompass 1-6 residues
  REGION 169..174: note = MISC_FEATURE - This region may encompass 1-6 residues
  REGION 175..180: note = MISC_FEATURE - This region may encompass 1-6 residues
  REGION 181..186: note = MISC_FEATURE - This region may encompass 1-6 residues
  REGION 187..192: note = MISC_FEATURE - This region may encompass 1-6 residues
  REGION 193..198: note = MISC_FEATURE - This region may encompass 1-6 residues
  REGION 199..204: note = MISC_FEATURE - This region may encompass 1-6 residues
  REGION 205..210: note = MISC_FEATURE - This region may encompass 1-6 residues
  REGION 211..216: note = MISC_FEATURE - This region may encompass 1-6 residues
  REGION 217..222: note = MISC_FEATURE - This region may encompass 1-6 residues
  REGION 223..228: note = MISC_FEATURE - This region may encompass 1-6 residues
  REGION 229..234: note = MISC_FEATURE - This region may encompass 1-6 residues
  REGION 235..240: note = MISC_FEATURE - This region may encompass 1-6 residues
  REGION 1..240: note = MISC_FEATURE - This sequence may encompass 3-20 "(Gly)i(Ser)j" repeating units wherein i ranges from 1 to 6 and j ranges from 1 to 6 and some positions may be absent
  REGION 1..240: note = See specification as filed for detailed description of substitutions and preferred embodiments
GGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSSGGGGGGSSSSSS

(19) United States
(12) Patent Application Publication
(10) Pub. No.: US 2024/0209390 A1
(43) Pub. Date: Jun. 27, 2024

(54) PLANT VIRUS MOVEMENT PROTEINS AND METHODS OF USING SAME

(71) Applicant: Molecular Transfer Inc., Carlsbad, CA (US)

(72) Inventors: Gulilat Gebeyehu, Potomac, MD (US); Joel Jessee, Mount Airy, MD (US)

(21) Appl. No.: 18/523,380

(22) Filed: Nov. 29, 2023

Related U.S. Application Data
(63) Continuation of application No. 16/747,443, filed on Jan. 20, 2020, now Pat. No. 11,866,723.
Application No. 16/747,443 is a continuation of application No. 15/446,578, filed on Mar. 1, 2017, now Pat. No. 10,538,784.
(60) Provisional application No. 62/303,278, filed on Mar. 3, 2016.
(60) Provisional application No. 62/302,155, filed on Mar. 1, 2016.

(51) Int. Cl. C12N 15/85; A61K 48/00; C07K 14/005; C07K 14/415; C12N 9/22; C12N 15/11; C12N 15/87; C12N 15/88; C12N 15/90

(52) U.S. Cl. CPC C12N 15/85; A61K 48/0041; C07K 14/005; C07K 14/415; C12N 9/22; C12N 15/11; C12N 15/87; C12N 15/88; C12N 15/907; C12N 2310/20; C12N 2810/10; C12N 2810/65

(57) ABSTRACT

Disclosed herein are transfection complexes comprising at least one cell surface ligand; at least one helper lipid component; and a transfection enhancer. Also disclosed are pharmaceutical compositions comprising the disclosed transfection complexes, and a pharmaceutically acceptable carrier. Further, disclosed are methods of transfecting a cell, the method comprising the steps of: obtaining a transfection complex as disclosed; and contacting a cell with the transfection complex.

### RELATED APPLICATIONS

The present application is a continuation of U.S. patent application Ser. No. 16/747,443, filed Jan. 20, 2020, which is a continuation of U.S. patent application Ser. No. 15/446,578, filed Mar. 1, 2017, now U.S. Pat. No. 10,538,784, issued Jan. 21, 2020, which claims priority to the U.S. Provisional Application Ser. No. 62/302,155, filed on Mar. 1, 2016, by Jessee et al., and entitled “PLANT VIRUS MOVEMENT PROTEINS AND METHODS OF USING THE SAME,” and U.S. Provisional Application Ser. No. 62/303,278, filed on Mar. 3, 2016, by Jessee et al., and entitled “PLANT VIRUS MOVEMENT PROTEINS AND METHODS OF USING THE SAME.” The entire disclosures of all of these applications are hereby incorporated by reference herein in their entireties.

### SEQUENCE STATEMENT

The Sequence Listing associated with this application is filed in electronic format via Patent Center and is hereby incorporated by reference into the specification in its entirety. The name of the xml file containing the Sequence Listing is 037595-539C02US_Sequence_Listing.xml. The size of the file is 754 kilobytes, and the file was created on Nov. 29, 2023.

### FIELD OF THE INVENTION

The present invention is in the field of transfection complexes suitable for the delivery of one or more biologically active agents to a cell and methods and kits for using the same.

### BACKGROUND OF THE DISCLOSURE

Lipid aggregates such as liposomes or cationic polymers can facilitate introduction of macromolecules, such as DNA, RNA, and proteins, into living cells. Aggregates comprising cationic lipid components can be used to effect delivery of large anionic molecules, such as nucleic acids, into certain types of cells.

The use of cationic lipids has become increasingly popular since their introduction over 25 years ago. Several cationic lipids have been described in the literature and some of these are commercially available. DOTMA (N-[1-(2,3-dioleyloxy)propyl]-N,N,N-trimethylammonium chloride; IUPAC: 1,2-di-O-octadecenyl-3-trimethyl-ammonium propane (chloride salt); CAS Number: 104872-42-6) was the first cationic lipid to be synthesized for the purpose of nucleic acid transfection. DOTMA can be formulated alone or can be combined with DOPE (dioleoylphosphatidylethanolamine) into a liposome, and such liposomes can be used to deliver plasmids into some cells. Other classes of lipids subsequently have been synthesized by various groups. For example, DOGS (5-carboxyspermylglycinedioctadecylamide) was the first polycationic lipid to be prepared and other polycationic lipids have since been prepared. The lipid DOSPA (2,3-dioleyloxy-N-[2(spermine-carboxamido)ethyl]-N,N-dimethyl-1-propanaminium) has been described as an effective delivery agent.

In other examples, cholesterol-based cationic lipids, such as DC-Chol (N,N-dimethyl-N-ethylcarboxamidocholesterol) have been prepared and used for transfection. Also, 1,4-bis(3-N-oleylamino-propyl)piperazine was prepared and combined with histone H1 to generate a delivery reagent that was reported to be less toxic than other reagents. Some examples of commercially available lipids include Lipofectin® (DOTMA:DOPE) (Thermo-Fisher, Carlsbad, CA), LipofectAminem (DOSPA:DOPE) (Thermo-Fisher), LipofectAmine2000™ (Thermo-Fisher), Lipofectamine 3000 (Therom-Fisher), Fugene®, Transfectam® (DOGS), ViaFect (Promega), DNA-In, Geneln (MTI-GlobalStem) Effectene®, and DC-Chol. Further examples are PEI polymers and dendrimers such as jetPEI (PolyPlus), and Superfect (Qiagen). None of these reagents can be used universally for all cells. This is perhaps not surprising in light of the variation in composition of the membranes of different types of cells as well as the barriers that can restrict entry of extracellular material into cells. Moreover, the mechanism by which cationic lipids deliver nucleic acids into cells is not clearly understood. The reagents are less efficient than viral delivery methods and are toxic to cells, although the degree of toxicity varies from reagent to reagent.

However, transfection agents, including cationic lipids, anionic lipids, cationic polymers, exosomes, and virasomes, are not universally effective in all cell types. Effectiveness of transfection of different cells depends on the particular transfection agent composition. In general, polycationic lipids are more efficient than monocationic lipids in transfecting eukaryotic cells. In many cases, cationic lipids alone are not effective or are only partially effective for transfection so helper lipids or transfection enhancers can be used in combination with cationic lipids.

Many biological materials are taken up by cells via receptor-mediated endocytosis, in which a surface ligand binds to a cell-surface receptor, leading to clustering of ligand-bound receptors, and formation of coated pits followed by internalization of the ligands into endosomes. Both enveloped viruses, like influenza virus and alphaviruses, and non-enveloped viruses, like Adenovirus, infect cells via endocytotic mechanisms. Enhancement of dendrimer-mediated transfection of some cells by chloroquine (a lysosomotropic agent) suggests that endocytosis is involved in at least some transfections.

Introduction of foreign DNA sequences into eukaryotic cells mediated by viral infection is generally orders of magnitude more efficient than transfection with anionic lipids, cationic lipid, PEI, peptides, or dendrimer transfection agents. Viral infection of all the cells in a culture requires fewer than 10 virus particles per cell. Although the detailed mechanism of fusion is not fully understood and varies among viruses, viral fusion typically involves specific fusogenic agents, such as viral proteins, viral spike glycoproteins and peptides of viral spike glycoproteins. Cell binding and internalization also can be enhanced, accelerated or made selective with peptides that bind cell receptors. For example, the penton-base protein of the Adenovirus coat contains the peptide motif RGD (Arg-Gly-Asp) which mediates virus binding to integrins and viral internalization via receptor-mediated endocytosis.

The efficiency of cationic lipid transfections has been shown to be enhanced by the addition of whole virus particles to the transfection mixture. Certain viral components may also enhance the efficiency of cationic lipid-mediated transfection. For example, it has been suggested that “Lipofectin™”-mediated transfections may be enhanced 3-4-fold by adding influenza virus hemagglutinin peptides to the transfection mixture. Antibodies have been shown to enhance cationic lipid transfections and transferrin-poly lysine or asialoglycoprotein polylysine have been shown to enhance cationic lipid transfection.

Nevertheless, these methods do not work for all cell types, require relatively complex protocols and are inconvenient. It is apparent, therefore, that new and improved methods for introducing macromolecules, and particularly nucleic acids, into cell, are greatly to be desired. In particular, improved methods for introducing nucleic acids into a wider variety of cells, and particularly into primary cells, are greatly to be desired.

### SUMMARY OF THE INVENTION

Disclosed herein are transfection complexes comprising at least one cell surface ligand or a plant virus movement protein or peptide fragments; at least one helper lipid component; and a transfection enhancer. Also disclosed are pharmaceutical compositions comprising the disclosed transfection complexes, and a pharmaceutically acceptable carrier. Further, disclosed are methods of transfecting a cell, the method comprising the steps of: obtaining a transfection complex as disclosed; and contacting a cell with the transfection complex.

### DETAILED DESCRIPTION OF THE EMBODIMENTS

### Definitions and Abbreviations

It is to be understood that the present invention is not limited to particular devices or biological systems, which may, of course, vary. It is also to be understood that, as used in this specification and the appended claims, the singular forms “a”, “an”, and “the” include singular and plural referents unless the content clearly dictates otherwise. Thus, for example, reference to “a lipid” includes one or more lipids. It is to be yet further understood that any terminology used herein is for the purpose of describing particular embodiments only, and is not intended to be limiting.

Unless stated otherwise, the following terms, definitions, and abbreviations as used herein are intended to have the following meanings:

As used herein, the term “labeled” is intended to mean that a compound has at least one element, isotope, or chemical compound attached to enable the detection of the compound by using a radioactive or heavy isotope label, or an immune label such as an antibody or antigen or a label derived from a colored, luminescent, phosphorescent, or fluorescent dye. Photoaffinity labeling employing, for example, o-, m- and p-azidobenzoyls, substituted with one or more halogen moieties, including, but not limited to 4-azido-2,3,5,6-tetrafluorobenzoic acid, is utilized for the direct elucidation of intermolecular interactions in biological systems.

The terms “subject” and “animal” are synonymous and, as used herein, refer to humans as well as non-human animals, including, for example, mammals (e.g., a rodent, a mouse, a rat, a rabbit, a monkey, a dog, a cat, a primate, or a pig), birds, reptiles, amphibians, and fish.

The term “cell” generally refers to eukaryotic cells of any type and from any source. Types of eukaryotic cells include epithelial, fibroblastic, neuronal, hematopoietic cells and the like from primary cells, tumor cells or immortalized cell lines. Sources of such cells include any animal such as human, canine, mouse, hamster, cat, bovine, porcine, monkey, ape, sheep, fish, insect, fungus, and any plant including crop plants, algae, ornamentals and trees.

“Delivery” is used to denote a process by which a desired compound is transferred to a target cell such that the desired compound is ultimately located inside the target cell or in, or on, the target cell membrane. In many uses of the compounds of the invention, the desired compound is not readily taken up by the target cell and delivery via lipid aggregates or transfection complexes a means for delivering the desired compound to the appropriate cellular compartment within a cell. In certain uses, especially under in vivo conditions, delivery to a specific target cell type is preferable and can be facilitated by transfection complexes comprising surface ligands of the invention.

Drug refers to any therapeutic or prophylactic agent other than food which is used in the prevention, diagnosis, alleviation, treatment, or cure of disease in man or animal.

“Kit” refers to transfection or protein expression kits which include one or more of the compounds of the present invention or mixtures thereof. Such kits may comprise a carrying means being compartmentalized to receive in close confinement one or more container means such as vials, test tubes and the like. Each of such container means comprises components or a mixture of components needed to perform transfection. Such kits may include one or more components selected from nucleic acids (preferably one or more vectors), cells, one or more compounds of the present invention, lipid-aggregate forming compounds, transfection enhancers, biologically active substances, etc.

The term “associated with”, when used in the context of molecular interactions, refers to two entities linked by a direct or indirect covalent or non-covalent interaction, such as hydrogen bonding, van der Waals interactions, hydrophobic interactions, magnetic interactions, electrostatic interactions, etc.

The term “biocompatible,” as used herein refers to compounds that are not toxic to cells. Compounds are biocompatible if their addition to cells in vitro results in less than or equal to 20% cell death, and their administration in vivo does not induce inflammation or other such adverse effects.

The term “biodegradable,” as used herein, refers to compounds that, when introduced into cells, are broken down into components that the cells can either reuse or dispose of without significant toxic effect on the cells (i.e., fewer than about 20% of the cells are killed when the components are added to cells in vitro). The components do not induce inflammation or other adverse effects in vivo. The chemical reactions relied upon to break down the biodegradable compounds are typically uncatalyzed. The term “effective amount,” as used herein with respect to an active agent, refers to the amount necessary to elicit the desired biological response. The effective amount of an agent or device may vary depending on such factors as the desired biological endpoint, the agent to be delivered, the composition of the encapsulating matrix, the target tissue, etc. Delivery of an “effective amount of a molecule” is the delivery of the molecule into a cell in sufficient amounts so that the molecule elicits a biological response, for example, modulating the expression of one or more genes in the cell. In specific embodiments, an effective amount of a molecule is delivered to a cell such that an amelioration or improvement in a disease, condition, or disorder related to the cell can be obtained. Delivery of an “effective amount of siRNA” or an “effective amount or RNAi” is the delivery of siRNA or other RNAi into a cell in sufficient amounts to cause a reduction in expression of the target gene in the cell.

The terms “biologically active agent”, “bioactive agents” or the like, generally refers to a composition, complex, compound or molecule which has a biological effect or that modifies, causes, promotes, enhances, blocks or reduces a biological effect, or that enhances or limits the production or activity of, reacts with and/or binds to a second molecules which has a biological effect. The second molecule can, but need not be, an endogenous molecule (e.g., a molecule, such as a protein or nucleic acid, normally present in the target cell). A biological effect may be, but is not limited to, one that stimulates or causes an immunoreactive response; one that impacts a biological process in a cell, tissue or organism (e.g., in an animal); one that imparts a biological process in a pathogen or parasite; one that generates or causes to generate a detectable signal; one that regulates the expression of a protein or polypeptide; one that stops or inhibits the expression of a protein or polypeptide; or one that causes or enhances the expression of a protein or polypeptide. Biologically active compositions, complexes, compounds or molecules may be used in investigative, therapeutic, prophylactic and diagnostic methods and compositions. Bioactive agents may include but are not limited to, pharmaceuticals, cell metabolites, proteins, nutrients (vitamins, amino acids, lipids, nucleotides, and carbohydrates), and exosomes.

The term “cationic lipid” refers to any cationic lipids which may be used for transfection and which under physiological conditions possess at least one positive charge. While it is to be understood that certain of the cell surface ligands that form the basis of the present disclosure can be formulated with cationic lipids the cationic lipids can be considered helper lipids.

The term “lysosomotropic agent” is any compound or molecule which inhibit lysosomal function that prevents or slows the acidification of the lysosomal compartment.

The term “nucleic acid binding moiety” as used herein refers to a compound or molecule capable binding to nucleic acid. In some embodiments, the binding molecule is capable of noncovalently binding to nucleic acid, while in other embodiments, the binding molecule links covalently to a cell binding adhesion sequence, a plant virus movement protein or peptide fragments, a nuclear localization sequence, transfection enhancer, and/or a fusion agent. The binding molecule can include but is not limited to spermine, spermine derivative, spermidine, histones or fragments thereof, protamines or fragments thereof, HMG proteins or fragments thereof, poly-lysine, poly-arginine, poly-histidine, polyamines and cationic peptides, nucleic acid intercalaters, protein nucleic acid sequences or aptamers. In addition, this includes but is not limited to analogs or derivatives of the above compounds. Non limiting examples are the cationic peptides that are repeats of lysine or arginine, for example a sequence having between 8-20 lysine residues (K8-K20) (SEQ ID NO:583) or between 8-20 arginine residues (R8-R20) (SEQ ID NO:584).

“Target cell” or “target tissue” refers to any cell or tissue to which a desired compound is delivered, using a lipid aggregate or transfection complex as carrier for the desired compound.

Transfection is used herein to mean the delivery of any nucleic acid, protein, peptide, lipid, cell nutrient, pharmaceutical agent, molecule or other macromolecule to a target cell or tissue in vitro or in vivo (i.e., in an animal, a plant or a human), such that the nucleic acid, protein or other macromolecule is expressed in, confers a phenotype to, causes enhanced growth, expression of a protein, or has a biological function in the cell.

The term “expressible nucleic acid” includes both DNA and RNA without regard to molecular weight, and the term “expression” means any manifestation of the functional presence of the nucleic acid within the cell including, without limitation, both transient expression and stable expression.

The term “fusion agent” as used herein refers to any chemical or molecules capable breaking down an endosomal membrane or cell membrane and freeing the transfection agent into the cytoplasm of the cell. This term includes but is not limited to viruses, synthetic compounds, proteins, fusion peptides, cell penetration peptides or proteins, or derivatives thereof. As a result of the presence of the fusion agent the membrane can undergo lysis, fusion, or rearrangement or all three.

The term “fusion peptide” refers to any peptide grouping which penetrates a membrane such that the structural organization and integrity of the membrane is lost. Fusion peptides are fusion agents.

The term “transfection agent” as used herein generally refers to composition capable of delivering molecules to cells. Transfection agents can be organic such as lipid, carbohydrate, cationic polymers, dendrimers, peptide or protein based or combination of those depending cell type or tissue that one targets. Transfection agents can also be in-organic such as calcium salts. They included cationic lipids, anionic lipids, cationic peptides, cationic proteins, polycationic virus hybrids, cationic polymers, exosomes, and combinations of the above. Transfection agent as used herein may optionally include at least one or more of the transfection compounds optionally in combination with one or more helper lipids, one or more pegylated lipids, one or more lipids from exosomes, complete lipid mixtures form exosomes, optionally one or more targeting moieties, optionally one or more cell surface ligands, optionally one or more nuclear localization sequences, optionally one or more fusion agents, optionally one or more condensing agents, optionally one or more cell penetration agents, optionally one or more plant movement proteins or peptide fragments, optionally one or more exosomes and optionally one or more lysosomotropic agents.

The term “transfection enhancer” as used herein refers to a compound when added to a transfection agent increases the efficiency of transfection (i.e., increases the percent of cells transfected), increases the level of expression of a transfection agent, or reduces the requirement for the amount of nucleic acid or protein required to give a biological response, or any combination of the enhancements above. In some embodiments, the transfection enhancer also helps deliver molecules that help downregulate expression such as siRNA, LNA's and the like.

The term “surface ligand” or “cell surface ligand” refers to a chemical or structure which will bind to a surface receptor of a cell. The term “cell surface receptor” as used herein refers to any specific chemical grouping on the surface of a cell to which the surface ligand can attach, contact or associate with. A surface ligand is a targeting moiety. Furthermore, surface ligands include anything which is capable of binding to the cell and centering the cell through cytosis (e.g., endocytosis, potocytosis, and pinocytosis).

The term “transfection complex”, as used herein generally refers to a composition formulated for the delivery of a biologically active agent, such as a nucleic acid, a protein, a macromolecule, cell nutrient, bioactive molecule or the like, to a cell or to a tissue in vivo or in vitro. Transfection complexes as used herein may include at least one or more of the transfection compounds or agents in combination with the biologically active compound to be delivered, optionally in combination with; one or more helper lipids, one or more pegylated lipids, one or more targeting moieties, one or more nuclear localization sequences, one or more fusion agents, one or more condensing agents, one or more cell penetration agents, one or one or more plant movement proteins or peptide fragments, complete exosomes, total lipid extracts isolated from exosomes, one or more exosome lipids, one more exosome protein components and one or more lysosomotropic agents in addition to the bioactive agent that is to be delivered. For the purposes described herein, the term “transfection complex” may be thought of as a lipoplex or a lipid aggregate contacted with a bioactive agent. Thus, in some instances in the following disclosure, terms such as lipoplex, lipid aggregate and the like may be used to make reference a transfection complex that lacks the one or more bioactive agents or “payloads”.

The term “helper lipid”, as used herein, generally refers to a lipid that is suitable for use in the preparation and formation of transfection complexes disclosed herein. Suitable helper lipids may include, though are not limited to DOPE, DPhPE, saturated and unsaturated DPPE, saturated and unsaturated DMPE, DOPC, Lyso-PE (1-acyl-2-hydroxy-sn-glycero-3-phosphoethanolamine), Lyso-PC (1-acyl-3-hydroxy-sn-glycero-3-phosphocholine), 3-alkyloxy-2-hydroxy-1-acetamidopropane, 4-alkyloxy-3-hydroxy-1-acetamidopropane, 5-alkyloxy-4-hydroxy-1-acetamidopropane, cholesterols, cholesterol derivatives, sterols, including phytosterols, zoosterols and hopanoids, or any of the neutral or cationic lipids that are known to allow or to facilitate the introduction of exogenous bioactive molecules to the interior of a cell or of a tissue. In some embodiments, more than one helper lipid may be used in the formulation of the transfection complexes described herein. Exemplary though non-limiting neutral or cationic lipids contemplated for use in the preparation of the presently disclosed transfection complexes may include one or more lipids selected from the following: N-(2-bromoethyl)-N,N-dimethyl-2,3-bis(9-octadecenyloxy)-propana minimun bromide (BMOP), dipalmitoylphosphatidylethanolamine 5-carboxyspermylamide (DDPES), DSPC, dioleoylphosphatidylethanolamine (DOPE), formulation of cetyltrimethylammonium bromide (CATB) and DOPE (CTAB:DOPE), 1-palmitoyl-2-oleoyl-sn-glycero-3-phosphocholine (POPC), DMG, 1,2-dimyristloxyaminopropane (DMAP), dimyristoylphospatidylethanolamine (DMPE), DOMG, DMA, Dioleoylphosphatidylcholine (DOPC), dimyristoylphosphatidylcholine (DMPC), Dipalmitoylethylphosphatidylcholine (DPEPC), dioleoydimethylammonium chloride (DODAC), 1,3-di-oleoyloxy-2-(6-carboxyspermyl)-propylamid (DOSPER), N-[1-(2,3-dioleyloxy)propyl]-N,N,N-trimethylammoniumchloride (DOTMA), N-[1-(2,3-dipalmitoleoyloxy)propyl]-N,N,N-trimethylammoniumchloride (DPTMA), didoceyl methylammonium bromide (DDAB), N-[1-(2,3-dioleoyloxy)propyl]-N,N,N-trimethyl-ammonium methylsulfate (DOTAP), DOTAP.C1, 3,β-N,(N′,N′-dimethylaminoethane)-carbamoyl]cholesterol (DC-chol), 2-(sperminecarboxamido)ethyl)-N,N-dimethyl-lammonium trifluoroacetate (DOSPA), O,O′-ditetradecanoyl-N-(alphatrimethylammonioacetyl) diethanolamine chloride (DC-6-14), dicaproylphosphtidylethanolamine (DCPE), dilauryl oxypropyl-3-dimethylhydroxy ethylammonium bromide (DLRIE), 1,2-dioleoyl-3-dimethylammonium-propane (DODAP), Ethyl-PC, 2,3-dioleoyloxy-N-[2-(sperminecarboxamidoethyl]-N,N-di-met-hyl-1-propanaminium trifluoroacetate (DOSPA), dioctadecylamidoglycyl carboxyspermine (DOGS), N-[1-(2,3 dimyristyloxy)propyl]-N,N-dimethyl-N-(2-hydroxyethyl) ammonium bromide (DMRIE), Dioleoylethyl-phosphocholine (DOEPC), N-[1-(2,3-dioleoyloxy)propyl]-N-[1-(2-hydroxyethyl)]-N,Ndimethylammonium iodide (DOHME), N-(3-aminopropyl)-N,N-dimethyl-2,3-bis(dodecyloxy)-1-propaniminium bromide/dioleylphosphatidylethanolamine (GAP-DLRIE.DOPE), dipalmitoylphospha-tidylcholine (DPPC), 1,2-dioleoyl-sn-glycero-3-[phospho-rac-(3-lysyl(1-glycerol)).Cl (DOPG), N-lauroylsarcosine, (R)-(+)-limonene, lecithins (and derivatives thereof); phosphotidylethanolamine (and derivatives thereof); phosphatidylethanolamines, dioleoylphosphatidylethanolamine), diphytanoylphosphatidylethanolamine (DPhPE), dipalmitoylphosphatidylethanolamine (DPPE), dipalmiteoylphosphatidylethanolamine, 3-β-[1-ornithinamidecarbamoyl]-cholesterol (0-Chol), palmitoyloleoylphosphatidyl-ethanolamine (POPE); distearoylphosphatidylethanolamine; phosphotidylcholine; phosphatidylcholines, dipalmitoylphosphatidylcholine (DPPC) palmitoyloleoyl-phosphatidylcholine (POPC); distearoylphosphatidylcholine; phosphatidylglycerol; piperazine-based cationic lipids, a phosphatidylglycerol, dioleoylphosphatidylglycerol (DOPG), dipalmitoylphosphatidyl-glycerol (DPPG), distearoylphosphatidylglycerol; phosphatidylserine (and derivatives thereof); phosphatidylserines, dioleoyl- or dipalmitoylphosphatidylserine; a diquaternary ammonium salt; N,N′-dioleyl-N,N,N′,N′-tetramethyl-1,2-ethanediamine (TmedEce), N,N′-dioleyl-N,N,N′,N′-tetramethyl-1,3-propanediamine (PropEce), N,N′-dioleyl-N,N,N′,N′-tetramethyl-1,6-hexanediamine (HexEce), and their corresponding N,N′-dicetyl saturated analogues (TmedEce, PropEce and HexEce), a diphosphatidylglycerol; a fatty acid ester; a monocationic transfection lipid; 1-deoxy-1-[dihexadecyl(methyl)ammonio]-D-xylitol; 1-deoxy-1-[methyl(ditetra-decyl)ammonio]-Darabinitol; 1-deoxy-1-[dihexadecyl(methyl)ammonio]-D-arabinitol; a 1-deoxy-1-[methyl(dioctadecyl)-ammonio]-darabinitol, glycerol ester; sphingolipids; cardolipin; a cerebroside; a ceramide, exosomes or lipids mixtures isolated from exosomes; and combinations thereof.

Helper lipids also include the neutral lipids cholesterol and other 3βOH-sterols, as well as derivatives thereof, phosphatidyl choline, or commercially available cationic lipid mixtures such as, for example, LIPOFECTIN® CELLFECTIN® (1:1.5 (M/M) formulation of N,N,N′,N″,N′″-tetramethyl-N,N,N′,N″,N′″-tetrapalmitylspermine (TMTPS) and dioleoyl phosphatidylethanolamine (DOPE), LIPOFECTACE®, GS 2888 CYTOFECTIN®, FUGENE 6®, EFFECTENE®, and LIPOFECTAMINE®, LIPOFECTAMINE 2000®, LIPOFECTAMINE PLUS®, LIPOTAXI®, POLYECT®, SUPERFECT®, TFXNT™, TRANSFAST™ TRANSFECTAM®, TRANSMESSENGER®, vectamidine (3-tetradecylamino-N-tert-butyl-N′-tetradecylpropionamidine (a.k.a. diC14-amidine), OLIGOFECTAMINE MessengerMAX, GeneIn™, TransfeX™, LipofectAmine 3000, Lipofectin®, DMRIE-C, CellFectin®, LipofectAce®, Fugene®, Fugene® HD, Tfx-10®, Tfx-20®, Tfx-50®, DNA-In, Transfectin™, SilentFect™, Effectene®, ViaFect™, DC-chol, GenePorter®, DharmaFect 1®, DharmaFect 2®, DharmaFect 3®, DharmaFect 4®, Escort™ III, Escort™ IV, DOGS among others. Also contemplated are any mixtures of combination of the above listed helper lipids, exosomes, and lipids mixtures isolated from exosomes.

Examples of lipids examples isolated from exosomes are include, but are not limited to, Lyso-PC (non-limiting examples include C-18, C-16, C-14 and mixture), lyso-bisphospahtidic acid (non-limiting example include C-18, C-16 and C-14), sphingomyelin, ceramides (non-limiting examples include C-8 and C-24), disaturated PC (non-limiting examples include DSPC, DPPC, DMPC, and compounds having a Cn length (where n=8-25), diunsaturated PC-MIX (non-limiting examples include DOPC and DP(db)PC), phosphatidyl serine (PS), phosphatidyl inositol (PI), disaturated PE (non-limiting example include DSPE, DPPE, and DMPE), di-unsaturated PE-MIX (non-limiting example include DOPE and DP(db)PE), posphatidyl glycerol (PG), (non-limiting examples include C-18-C-22), cholesterol, and diglycerides, such as cardiolipin.

Also contemplated are any mixtures of combination of the above listed helper lipids, exosomes, and lipids mixtures isolated from exosomes.

The following patent documents, patent applications, or references are incorporated by reference herein in their entirety and in particular for their disclosure of transfection agents containing cationic and neutral helper lipids, which may be used in the transfection complexes disclosed herein: U.S. Pat. Nos. 6,075,012; 6,020,202; 5,578,475; 5,736,392; 6,051,429; 6,376,248; 5,334,761; 5,316,948; 5,674,908; 5,834,439; 6,110,916; 6,399,663; 6,716,882; 5,627,159; 7,915,230; 7,531,693; 8,034,977; 7,166,745; 5,994,109; 6,033884; 6,150,168; 6,177,554; 6,083,741 6,458,026; 7,598,421; 7,820,624; 7,256,043; 7,704,969; 8,026,341; 7,145,039; 7,531,693; and 8,785,200; and International Publications WO 2004/063342, WO 0027795, WO 2004/105697, WO 2007/130073, WO 2012/142622, and WO 2013/158127,

The term “pegylated lipid” as used herein generally refers to a lipid that is covalently conjugated to one or more polyethylene glycol moieties. Pegylated lipids for lipoplex embodiments herein include phosphatidylethanolamine (PE) based pegylated lipids such as, for example, 1,2-dimyristoyl-sn-glycero-3-phosphoethanolamine-N-[methoxy(polyethylene glycol)-MW] where MW refers to average MW of the polyethylene glycol moiety. Such dimyristoyl-PEG-PE lipids are commonly designated 14:0 PEG (MW) PE. The average MW of the polyethylene glycol moiety can be 25, 350, 550, 750, 1000, 2000, 3000, 5000, 6000, 8000 or 12000, for example. The fatty acid chains of the phosphatidylethanolamine based pegylated lipids may include, for example, a 1,2-dioleoyl group such as for 18:1 PEG (MW) PE, a 1,2-dipalmitoyl group such as for 16:0 PEG (MW) PE, or a 1,2-distearoyl-group such as for 18:0 PEG (MW) PE. Further phosphatidylethanolamine (PE) based pegylated lipids include, for example, 1,2-distearoyl-sn-glycero-3-phosphoethanolamine-N-[MOD(polyethylene glycol)-MW], also referred to as DSPE-MOD PEG (MW) wherein MOD refers to a functional moiety such as an amine, biotin, carboxylic acid, folate, maleimide, PDP, or carboxyfluorescein moiety. The MW may be 2000 or 5000, for example. Pegylated lipids for the embodiments described herein also include ceramide based pegylated lipids such as, for example, N-octanoyl-sphingosine-1-{succinyl[methoxy(polyethylene glycol)MW]}, designated C8 PEG (MW) ceramide, where MW is 750, 2000, or 5000, for example. Alternatively, the fatty acid moiety may have an N-palmitoyl (C16) group such as for C16 PEG (MW) ceramide.

A “liposomal composition” generally is a formulation that includes one or more liposomes. In some instances, the term “liposomal composition” may be used interchangeably with the term “transfection complex”. These formulations are typically colloids, but can be dried formulations as well. A liposome is a vesicular colloidal particle composed of self-assembled amphiphilic molecules. Surface ligands, plant virus movement proteins, or fragments thereof disclosed herein can be incorporated into liposomal compositions of one or more cationic lipids, or one or more anionic lipids either or one or more pH sensitive lipids alone or optionally in combination with one or more helper lipids (i.e., a neutral lipid, a cholesterol or cholesterol derivative, lysolipid. or cationic lipids) that are processed using standard methods to form a liposome-containing colloid suspension. Liposomal compositions disclosed herein are those containing one or more cationic lipids, one or more helper lipids, optionally, in combination with one or more neutral and/or helper lipids, exosomes, total lipid extract from exosomes, targeting moieties, fusion agents, cell penetration agents, lysomotropic agents which are treated by any of the standard methods known in the art without limitation to form liposomes. The liposomal compositions may optionally contain one or more fusion agents. The liposomal compositions may optionally contain one or more liposomal compositions can be distinguished one from another by particle size measurements. Different compositions will exhibit differences in particle size and uniformity of particle size, e.g., average particle size, and polydispersity. Different compositions will exhibit differences in the extent of the composition that is in the form of liposomes. In some non-limiting embodiments, liposomal compositions will exhibit particle size in the range 120 nm and 800 nm and will exhibit generally lower polydispersity. Lipoplex particle size (with siRNA or other cargo) may range from about 40 nm to 135 nm. In some embodiments, lipoplex particle size is 50 nm to 120 nm, 50 nm to 100 nm, 60 nm to 90 nm, 70 nm to 90 nm, or about 85 nm.

The term “Lipid aggregate” or “lipoplex” is a generic term that includes liposomes of all types, both unilamellar and multilamellar, as well as vesicles, micelles, exosomes, micro-vesicles and more amorphous aggregates. A cationic lipid aggregate is a lipid aggregate comprising a combination of one or more cationic compounds, optionally in combination with non-cationic lipids (including neutral lipids), exosomes, such that the lipid aggregate has a net positive charge. Surface ligands or plant virus movement proteins or fragments thereof disclosed herein can be incorporated into lipid aggregate, optionally with a helper lipid and further optionally with one or more pegylated lipids and/or one or more targeting moieties, one or more fusion agents, one or more cell penetration agents and one or more lysosomotropic agents, one or more exosomes, which can then form a lipid-bioactive agent complex when contacted with a suitable bioactive agent. The terms “lipid aggregate” or “lipoplex” are generally used herein to refer to a “naked” transfection complex, i.e., a transfection complex that generally lacks a payload of bioactive agent to be delivered to a cell or to a tissue in vitro or in vivo.

The term “exosome” refers to the small membrane vesicles secreted by most cells that contain cell specific payloads of proteins, lipids and, genetic material and other biomolecules that are transported to other cells in different location of the tissue. Exosomes can be considered liposomal particles. Exosomes or lipid mixtures obtained therefrom, can be used in combination with other transfection agents or helper lipid mixtures. Exosomes are also referred to as microvesicles, epididimosomes, argosomes, exosome-like vesicles, microparticles, promininosomes, prostasomes, dexosomes, texosomes, archeosomes and oncosomes In one example of lipid constituents of exosomes is Lyso-PC (non limiting examples of which C-18, C-16, C-14 and mixture), Lyso-bisphospahtidic acid (non limiting example of which is C-18, C-16 and C-14), Sphingomyelin, Ceramides (non limiting examples C-8-C-24), Disaturated PC (non limiting examples (DSPC, DPPC, DMPC and others where Cn (n=8-25) Diunsaturated PC-MIX (non limiting examples of which are DOPC, DP(db)PC) phosphatidyl serine (PS), phosphatidyl inositol (PI), Disaturated PE (non limiting example, DSPE, DPPE, DMPE), Di-unsaturated PE-MIX (non limiting example DOPE DP(db)PE), posphatidyl glycerol (PG), (non limiting examples of which are C-18-C-22, Cholesterol, Diglycerides such as cardiolipin

The term “lipid-bioactive agent” generally refers to the noncovalent association between a lipid or lipid aggregate and a bioactive agent, such as a nucleic acid, nucleotide, amino acid, peptide, a polypeptide, protein, protein nucleic complex, nutrient, exosome and the like.

As used herein “nucleic acid” and its grammatical equivalents will include the full range of polymers of single or double stranded nucleotides and includes nucleic acids (including DNA, RNA, and DNA-RNA hybrid molecules, Linked Nucleic acids (LNA), Bridged Nucleic acid (BNA)) that are isolated from a natural source; that are prepared in vitro, using techniques such as PCR amplification or chemical synthesis; that are prepared in vivo, e.g., via recombinant DNA technology; or that are prepared or obtained by any known method. A nucleic acid typically refers to a polynucleotide molecule comprised of a linear strand of two or more nucleotides (deoxyribonucleotides and/or ribonucleotides) or variants, derivatives and/or analogs thereof. The exact size will depend on many factors, which in turn depends on the ultimate conditions of use, as is well known in the art. The nucleic acids of the present invention include without limitation primers, probes, oligonucleotides, vectors, constructs, plasmids, genes, transgenes, genomic DNA, cDNA, LNA, BNA, RNA, mRNA, tRNA, miRNA, RNAi, siRNA, shRNA, stRNA, guide-RNA, gBlock, PCR products, restriction fragments, oligonucleotides and the like.

As used herein, the term “nucleotide” includes any monomeric unit of DNA or RNA containing a sugar moiety (pentose), a phosphate, and a nitrogenous heterocyclic base and may also include mono-, di- and triphosphate forms of such nucleotides. The base is usually linked to the sugar moiety via the glycosidic carbon (at the 1′ carbon of pentose) and that combination of base and sugar is called a “nucleoside.” The base characterizes the nucleotide with the four customary bases of DNA being adenine (A), guanine (G), cytosine (C) and thymine (T). Inosine (I) is an example of a synthetic base that can be used to substitute for any of the four, naturally occurring bases (A, C, G, or T). The four RNA bases are A, G, C, and uracil (U). Accordingly, a nucleic acid may be a nucleotide sequence comprising a linear array of nucleotides connected by phosphodiester bonds between the 3′ and 5′ carbons of adjacent pentoses. Other modified nucleotides are known and may be sued in the practice of the invention. The term nucleotide includes ribonucleoside triphosphates ATP, UTP, ITP, CTG, GTP or derivatives such as but not limited to [αS] ATP, 7-deaza-GTP and 7-deaza-ATP, 5-methyCTP, pseudoUTP, 4-thioUTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, 5-methydCTP, pseudodUTP, 4-thiodUTP, LNA-Nucleosidetriphosphates and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. The term nucleotides as used here also refer to nucleotides that contain modifiable groups. Illustrated examples of nucleotides with modifiable group include, but are not limited to, allyamine-CTP, allyamine dCTP, allyamine UTP, allyamine dUTP. According to the present invention, a “nucleotide” may be unlabeled or detectably labeled by well-known techniques. Detectable labels include, for example, biotin, radioactive isotopes, fluorescent labels, chemiluminescent labels, bioluminescent labels and enzyme labels. Various labeling methods known in the art can be employed in the practice of this invention. Transfection complexes of this invention can be used to deliver nucleotides to living cells to allow incorporation of modified nucleotides in nucleic acids.

“RNA” or “RNA molecule” refers to any RNA molecule or functional portion thereof, of any size, self-replicating, and having any sequence, from any source, including RNA viral, prokaryotic, and eukaryotic organisms. The RNA molecule may be chemically modified and in any form, including, but not limited to, linear or circular, and single or double stranded. Non-limiting examples of RNA molecules include mRNA, rRNA, tRNA, miRNA, mtRNA, tmRNA, RNAi, siRNA, shRNA, guideRNA, and stRNA. In some embodiments, siRNA molecules useful in the practice of the invention include, for example, those described in U.S. Patent Publication Nos. 2004/0014956, 2004/0054155, 2006/0009409, 2009/0023216, and 2010/0136695; and as described in International Publications WO 2003/064626, and WO 03/064625, all of which are incorporated by reference herein. Further siRNA molecules useful in the practice of the invention include, for example, those described in International Publication WO 2009/039173, which application is incorporated by reference herein.

The terms “peptide”, “polypeptide”, or “protein,” as used herein refer to a string of at least three amino acids linked together by peptide bonds. The terms “protein” and “peptide” may be used interchangeably, though it is generally understood that a “polypeptide” or “protein” is larger than a peptide. “Peptide” may refer to an individual peptide or a collection of peptides.

The terms “polynucleotide” or “oligonucleotide,” as used herein, refer to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). The term “lipid” refers to hydrophobic or amphiphilic organic compounds inclusive of fats, oils and triglyderides.

### Disclosed Aspects and Embodiments

Transfection complexes suitable for the delivery of one or more biologically active agents to a cell or a tissue in vitro or in vivo are provided for herein. The transfection complexes described herein include one or more cell surface ligands, plant virus movement proteins, or fragments thereof that enhance transfection in combination with a transfection agent as part of a transfection complex. In some embodiments, the transfection complexes disclosed herein optionally further comprise one or more components selected from the group consisting of one or more helper lipids, one or more pegylated lipids, one or more cationic lipids, one or more cationic polymers one or more targeting moieties, exosomes, total lipid isolated from exosomes, total lipid and protein isolated form exosomes, a combination thereof. In some embodiments, transfection complexes disclosed herein further comprise one or more components selected from the group consisting of peptide or non-peptide transfection enhancers, consisting of peptide or non-peptide surface ligand, cell penetration peptide or non-peptide cell penetration agent, fusogenic peptide or non-peptide fusion agents, peptide or non-peptide endosomal release agents, lysomolotropic agents, nuclear targeting agents (such as, e.g., a peptide containing one or more nuclear localization sequences), and a combination thereof.

Plant virus movement proteins are involved in moving the genome of plant viruses from cell to cell and from cell membrane to nucleus and back to the cell membrane and then into the neighboring cells using the plant host cell machinery in combination with the plant virus movement protein. All plant viruses have movement protein but no all have been studied. These virus movement proteins, or fragments of such have utility for moving DNA or RNA in eukaryotic cells from the cell cytoplasium to the nucleus or nucleus to cell membrane. The virus genera and virus families of Tobamovirus, Dianthovirus, Umbravirus, Bromovirus, Cucumovirus, Begomovirus, Potyvirus, Hordei-like virus, Potex-like virus, Begomoviruses, Geminiviridae have example of virus movement proteins that move nucleic acid to and from the cell membrane and to the nucleus if a DNA virus. Utilization of these plant proteins to enhance transfection of both DNA and RNA are disclosed.

In some embodiments, helper lipids suitable for use in the preparation and formation of transfection complexes disclosed herein include, though are not limited to a cholesterol, a cholesterol derivative, one or more sterols, including phytosterols, zoosterols and hopanoids, or any of the neutral or cationic lipids that are known to allow or to facilitate the introduction of exogenous bioactive molecules to the interior of a cell or of a tissue. The helper lipid or helper lipids may be derived form exosomes or complete exosomes. In some embodiments, more than one helper lipid is used in the formulation of the transfection complexes described herein. In some embodiments, the transfection complexes disclosed herein comprise no helper lipid.

Illustrative though non-limiting neutral or cationic lipids suitable for use as helper lipids in accordance with some of the embodiments set forth herein include saturated and unsaturated alkyl and alicyclic ethers and esters of amines, amides or derivatives thereof. In some embodiments, straight-chain or branched alkyl and alkene groups of cationic lipids contain from 1 to about 25 carbon atoms. In certain embodiments, straight-chain or branched alkyl or alkene groups have six or more carbon atoms. In some embodiments, straight-chain or branched alkyl or alkene groups have eight to about twenty carbon atoms. In other embodiments, alicyclic groups contain from about 6 to 30 carbon atoms, or, alternatively, eight to twenty carbon atoms. In some embodiments, the alicyclic groups include cholesterol and other steroid groups. In certain embodiments, cationic lipids are prepared with a variety of counter ions (anions) including among others: a halide (i.e., Cl⁻, Br⁻, I⁻, F⁻), acetate, trifluoroacetate, sulfate, nitrite, triflate, and nitrate

Embodiments of pegylated lipids suitable for use in the preparation and formation of the transfection complexes disclosed herein are any lipid or mixture of lipids that are compatible with the formation of transfection complexes described herein, and with the administration thereof to an animal or to a human in vivo, or to tissues or cells in vitro. The pegylated lipids used with the presently described transfection complexes include, but are not limited to, a PEG polymer having a molecular weight between about 250 daltons and about 12,000, or in some embodiments, about 350 daltons and about 6,000 daltons, or, in some embodiments, between about 500 daltons and about 1,000 daltons, or, in some embodiments, between about 1,000 daltons and about 2,000 daltons, or, in some embodiments, between about 2,000 daltons and 5,000 daltons.

In some embodiments, the presently disclosed transfection complexes include one or more biologically active agents to be delivered to a cell or to a target tissue in vitro or in vivo. Suitable biologically active agents include, but are not limited to, any molecule that is capable of forming a transfection complex with the presently described transfection reagents and that elicits a biological response when delivered to the interior of a cell or cells or to a tissue in vivo or in vitro. In some embodiments, biologically active agents contemplated for use in the presently described embodiments are cationic, neutral or anionic agents. In some embodiments, the biologically active agents suitable for formulation in the presently disclosed transfection complexes include a protein, DNA or RNA molecule, either alone or in combination with other protein, DNA or RNA molecules in various combinations, though are not limited to; nucleic acids (including but not limited to single or double stranded linear or circular DNA molecules including cDNA molecules, single or double stranded RNA molecules, mRNA, modified mRNA that has increase stability, small interfering RNA (siRNA) molecules, small hairpin RNA (shRNA) molecules, guideRNA (gRNA), Cas9 protein, Cas9 protein/guide RNA, Cas9DNA/guideRNA, Cas9mRNA/guideRNA, Cas9mRNA/gRNA/ssDNA/RecAprotein, Cas9mRNA/gRNA/ssDNA/recombination protein, Cas9protein/gRNA/ssDNA/RecAprotein, Cas9protein/gRNA/ssDNA/recombination protein, microRNA (miRNA) molecules, oligonucleotides, anti-sense oligonucleotides, sense oligonucleotides), polypeptides, antibodies, oligopeptides, therapeutic peptides or protein molecules, peptide nucleic acids (PNAs), cationic, anionic or neutral organic molecules or drugs, in addition to pharmaceutically acceptable salts thereof. In another embodiment, nutrients required for cell growth or nutrients that can be used to enhance protein expression can be delivered into cells by transfection complexes disclosed herein.

In certain non-limiting illustrative embodiments, the transfection complexes disclosed herein deliver nucleic acid molecules into cells or tissues in vitro or in vivo, including the delivery of RNA interference molecules (RNAi) or small interfering RNA molecules (siRNA, shRNA or miRNA) into cells for inhibition of gene expression.

In some embodiments, the cell surface ligands, the plant virus movement proteins, peptide fragments thereof, or the presently disclosed transfection complexes are used to deliver mRNA molecules or mixtures of mRNA and DNA molecules into a cell or a tissue in vivo or in vitro to promote the expression of a specific protein or proteins. mRNA reprograming molecules or telomerase are non-limiting examples of mRNA molecules. Cas9 mRNA, and DNA molecules that code for gRNA, CRE mRNA and LoxP containing DNA molecules, SV40T antigen mRNA and DNA molecules with the SV40 origin of replication are non-limiting examples of mRNA and DNA pairs that have utility. Preformed transfection complexes that contain mRNA are non-limiting illustrative embodiments of transfection complexes disclose herein. Telomerase mRNA transfection complexes or Telomerase mRNA and SV40 Large T-antigen complexes as a media supplement are disclosed herein. In some embodiments, preformed transfection complexes containing mRNA are made and the DNA molecule is added to the complex at a later time.

In some embodiments, the cell surface ligands, plant virus movement proteins or peptide fragments thereof, or the presently disclosed transfection complexes are used to deliver DNA molecules (including cDNA molecules) into a cell or a tissue in vivo or in vitro to promote the expression of a specific protein or proteins or to synthesize specific RNA molecules, including but not limited to mRNA molecules or RNAi or miRNA or shRNA or sgRNA molecules are also provided.

In some embodiments, the cell surface ligands, plant virus movement proteins, or peptide fragments thereof presently disclosed, are used to deliver proteins or protein nucleic acid complexes into a cell or a tissue in vivo or in vitro to effect the function of the protein, as for example in gene editing. In some embodiments, the transfection complexes described herein contain, one or more surface ligands, one or more fusogenic peptides, one or more nuclear targeting peptide, one or more cationic lipid, or one or more neutral lipid. Non-limiting examples of proteins and protein nucleic acid complexes include, recombinases, CRISPR enzymes, Cre recombinase and Cre fusion proteins, transcription activator like effector nucleases (TALEN), genome editing proteins and CRISPR-Cas9 nuclease/guide RNA complex.

In some embodiments, proteins such as RNA polymerase, RNA binding proteins or peptides, and transcription factors are bound to nucleic acids and are delivered to cells with the transfection reagents disclosed herein. In certain embodiments, proteins are made anionic by the addition anionic peptides or anionic polymers designed to attach to the protein or an anionic amino acid is added to the C-terminus or N-terminus of the protein.

In some embodiments, the transfection complexes described herein may optionally include one or more fusogenic or cell-penetrating peptides. A fusogenic or cell-penetrating peptide is any peptide molecule that is capable of promoting the fusion of a lipid-containing complex to a cell membrane (either a plasma membrane or an endosomal membrane). A variety of fusogenic or cell-penetrating peptides are known in the art and it is well within the skill level of a practitioner to identify suitable fusogenic or cell-penetrating peptides and condition for the use thereof in the present invention without undue experimentation.

In some embodiments, the transfection complexes described herein optionally include one or more transfection helpers or targeting moieties in combination with the cell surface ligands, plant virus movement proteins, or peptide fragment thereof described herein. In some embodiments, the targeting moiety is a peptide, a modified peptide, an antibody, a modified antibody, a receptor molecule, a modified receptor molecule, a single or a double stranded nucleic acid molecule, a modified single or double stranded nucleic acid molecule, a peptide or nucleic acid aptamer, a modified peptide or nucleic acid aptamer, an organic molecule, a polysaccharide, an exosome, or any other molecule that is capable of targeting a transfection complex to specific tissue or cell type for targeted delivery of a biologically agent thereto, such as will be readily apparent to those having ordinary skill level in the art. In some embodiments, modification of a peptide, an antibody, a nucleic acid, an aptamer, and the like includes conjugating the peptide, antibody, nucleic acid, aptamer, and the like to a PEG moiety. Alternatively, said modification includes conjugating the peptide, antibody, nucleic acid, aptamer, and the like to a PEG-lipid moiety. A variety of targeting moieties are widely known to those skilled in the art, and all are contemplated for use with the presently described embodiments, without limitation.

In some embodiments, the transfection complexes disclosed herein are stable for up to one year and are either contacted with the cells or tissues to be transfected, or are administered to a subject immediately or shortly after being formed. In some embodiments, the transfection complexes disclosed herein are optionally stored for a period of time prior to being contacted with the cells or tissues, or being administered to a subject. The transfection complexes are stable and may be stored for a time period of at least 30 minutes, at least 45 minutes, at least 1 hour, at least 2 hours, at least 3 hours, at least 4 hours, at least 5 hours, at least 10 hours, at least 15 hours, at least 20 hours, at least 24 hours, at least 48 hours, at least 72 hours, at least 5 days, at least 7 days, at least 14 days, at least 28 days, at least 1 month, at least 2 months, at least 3 months, at least 4 months, at least 5 months, at least 6 months or at least 1 year at room temperature, or at a temperature greater than freezing, up to about room temperature. In some embodiments, the formulations described herein include one or more stabilizing agents, preservatives, buffers, and the like, that aid in the long-term stabilization and storage of bioactive formulation, such as will be readily understood by the skilled practitioner of the biological and pharmaceutical arts, and without requiring undue experimentation to achieve. It is also understood, that the storage period can be between any of the aforesaid time periods, for example between 31 minutes and 1 hour or between 1 hour and 24 hours.

In another aspect, disclosed herein are methods for the preparation of functional transfection complexes containing cell surface ligands, plant virus movement proteins, or peptides derived thereform, the complexes being as described herein. In some embodiment the functional transfection complex may be used for the preparation of synthetic liposomes or exosomes that contain biologically active macromolecules. In some embodiments, the methods include the step of forming a liposomes or exosome composition containing one or more plant virus movement proteins or peptide fragments or from surface ligands described herein, optionally in combination with one or more helper lipids, stabilizing lipids, transfection helpers, exosomal lipids or lipid extracts, pegylated lipids, targeting moieties, fusion agents, cell penetration agent, lysosomotropic agent. In some embodiments, the methods include the step of forming a lipid-aggregate by encapsulating a biologically active agent in a composition containing one or more surface ligands, plant virus proteins, or peptide fragments thereof described herein, optionally in combination with one or more helper lipids, stabilizing lipids, transfection helpers, pegylated lipids, targeting moieties, fusion agents, lysosomotropic agent and/or exosomes. In some embodiments, the methods alternatively include: 1) mixing one or more surface ligands, plant virus movement proteins, or peptide fragments thereof, with one or more transfection compounds, which optionally include one or more helper lipids, exosomes, surface ligands, stabilizing lipids, transfection helpers, targeting moieties, fusion agents, lysosomaotropic agent, optionally with one or more pegylated lipids, or a salt thereof, in an, an aqueous, alcohol/aqueous, or alcohol solution wherein the alcohol concentration is <10%, <25%, <50%, or <99%; 2) mixing one or more surface ligands, plant virus movement proteins, or peptide fragments thereof, with one or more transfection compounds, which optionally include one or more helper lipids, exosomes, stabilizing lipids, transfection helpers, targeting moieties, surface ligands, fusion agents, cell penetration agents, lysosomotropic agent, and one or more pegylated lipids, or a salt thereof, in a molar percentage such that the one or more transfection compounds are present at 1%-90%; 3) mixing one or more surface ligands, plant virus movement protein, or peptide fragment thereof, with one or more transfection compounds, which optionally include one or more helper lipid, exosomes, stabilizing lipids, transfection helpers, targeting moieties, surface ligands, fusion agents, lysosomotropic agent, one or more pegylated lipids, or a salt thereof, in a molar percentage such that the Pegylated lipids are present at <50%; and 4) mixing one or more surface ligands, plant virus movement proteins, or peptides derived therefrom, with one or more transfection compounds, which optionally include one or more helper lipid, exosomes, stabilizing lipids, transfection helpers, targeting moieties, surface ligands fusion agents, cell penetration agents, lysosomotropic agent, one or more pegylated lipids, or a salt thereof, wherein the pegylated lipid has a polyethylene glycol molecular weight of about 2000-12000 and a fatty acid chain length of C₆-C₂₀ alkyl, or C₁₀-C₂₀ alkenyl; and complexing the lipid aggregate in an aqueous, alcohol/aqueous, or alcohol solution with the bioactive agent to form a transfection complex, wherein the alcohol concentration is <50%, preferably less than 40% if pegylated lipids are present. In some embodiments, the alcohol is ethanol. In some embodiments, the alcohol is a pharmaceutically acceptable alcohol such as an alcohol that is liquid at about room temperature, for example, ethanol, propylene glycol, 2-(2-ethoxyethoxy)ethanol (Transcutol™), benzyl alcohol, glycerol, polyethylene glycol 200, polyethylene glycol 300, polyethylene glycol 400 or a mixture thereof. In some embodiments, the alcohol for mixing is different than the alcohol for complexing. Formulations of lipid aggregates as provided above can be provided in 0% to 100% ethanol. In some embodiments, the helper lipid is a neutral lipid. The ratio of cationic to neutral lipid can vary from 100% to 0.1% cationic lipid.

In another embodiment preformed lipid aggregates, exosomes, cationic exosomes or liposomes containing cationic, anionic, and or neutral lipids are mixed with cell surface ligands, plant virus movement proteins, or peptides derived therefrom describe herein. In certain embodiments, the lipid aggregates, exosomes or liposomes are optionally formulated with one or more transfection enhancers such as helper lipids, stabilizing lipids, transfection helpers, pegylated lipids, other targeting moieties, cell surface ligands, fusion agents, cell penetration agents, and or lysosomotropic agents. In some embodiments, the cell surface ligands, plant virus movement proteins, or peptides derived therefrom as described herein are added to these formulations at any point prior to or after the bioactive agent is loaded into the transfection complex.

In another embodiment a mixture of cationic lipid, neutral lipids and or total lipid extract of exosomes are dissolved in organic solvent such as chloroform and mixed with aqueous solutions optionally containing buffers and one or more cell surface ligands, plant virus movement proteins, or peptides derived therefrom, and optionally one or more transfection enhancers, one or more fusion agents, one or more cell penetration agents, one or more nuclear localization agents and subjected to reveres evaporation to remove the organic solvent leaving behind a lipid aggregate or liposome in solution.

In another embodiment a mixture of cationic lipid, neutral lipids and or total lipid extract of exosomes are dissolved in organic solvent such as chloroform or ethanol and mixed with aqueous solutions optionally containing buffers and one or more plant virus movement proteins or peptides derived from plant virus movement proteins, and optionally one or more transfection enhancers, one or more fusion agents, one or more nuclear localization agents, one or more cell surface ligand and subjected to reveres evaporation to remove the organic solvent leaving behind a lipid aggregate or liposome in solution.

In another embodiment, a mixture of cationic lipids and, lipids (total lipid extract of exosomes, lipids from exosomes, neutral lipids) in a water, alcohol or in a alcohol water mixture is added to an alcohol solution or alcohol water solution containing buffers and plant virus movement proteins or peptides derived from plant virus movement proteins, and optionally one or more transfection enhancers, one or more exosomes, one or more fusion agents, one or more nuclear localization agents, one or more surface ligands and this solution is optionally micro fluidization, extruded or sonicated to form lipid aggregates or liposomes.

In another embodiment, a mixture of cationic lipids and neutral lipid in a water, alcohol or in an alcohol/water mixture is added to an aqueous solution containing buffers and one or more cell surface ligands, plant virus movement proteins, or peptides derived therefrom, and optionally one or more transfection enhancers, one or more exosomes, one or more fusion agents, one or more cell penetration agents, one or more nuclear localization agents one or more surface ligands and this solution is optionally micro fluidization, extruded or sonicated to form lipid aggregates or liposomes.

In another aspect, disclosed herein are methods for screening for a tissue-based delivery of a transfection complex or cell type. In some embodiments, the method comprises the step of preparing a plurality of transfection complexes, each transfection complex having at least one cell surface ligand, plant virus movement protein, or peptides derived therefrom, in combination with at least one nucleic acid that facilitates detection of delivery to a tissue or cell type. In some embodiments, the nucleic acid is an RNA molecule or a DNA molecule that encodes a protein that can be directly detected (such as, e.g., Green Fluorescent Protein (GFP), Red Fluorescent Protein, Luciferase, or the like), or encode a protein that effects expression of a protein that can be directly detected.

In an embodiment, a method for screening for a cell based or tissue-based delivery of at least one cell surface ligand, plant virus movement protein, or peptide derived therefrom, comprises the step of preparing a plurality of unique transfection complexes, each transfection complex having at least cell surface ligand, plant virus movement protein, or peptide derived therefrom in combination with an mRNA or a cDNA that encodes the detectable protein or a specific transcription factor. Each unique transfection complex is delivered either to cells in culture, intravenously, subcutaneously, or to a tissue to a subject. After a predetermined amount of time, cells in culture or tissues from the subject are harvested and the expression of the detectable protein in various tissues is detected by gross examination, histological examination or by molecular detection (PCR, Western blotting, or the like), or imaged in vivo using the IVIS® Imaging System (Caliper), to determine to which tissue or tissues the transfection complexes containing specific transfection compounds are delivered.

In an embodiment, a method for screening cells in culture or tissue-based delivery of a transfection complex comprises the step of preparing a plurality of unique transfection complexes, each transfection complex having at least one test cell surface ligand, plant virus movement protein, or peptide derived therefrom in combination with an mRNA or a cDNA that encodes a specific transcription factor. Each unique transfection complex may be delivered to cells in culture, intravenously, subcutaneously, or to a tissue to a transgenic animal that expresses a reporter gene (such as, e.g., luciferase) under the control of the specific transcription factor. After a predetermined amount of time, tissues from the transgenic animal may be harvested and the expression of reporter gene in various tissues may be detected by gross examination, histological examination or by molecular detection (PCR, Western blotting, or the like). If the reporter gene is luciferase, detection may be accomplished in-vivo using the IVIS® Imaging System (Caliper).

In some embodiments, cell surface ligand, plant virus movement protein, or peptide derived therefrom of the presently disclosed transfection complexes are used to deliver exosomes into a cell or a tissue in vivo or in vitro to effect the function of the biological cargo in the exosomes. In some embodiments, the transfection complex described herein comprise one or more fusogenic peptides, one or more cell penetration agents, one or more nuclear targeting peptide, one or more cationic lipid, one or more plant virus movement proteins or peptides derived from plant virus movement proteins, one or more surface ligands or one or more neutral lipids. In certain embodiments, the transfection complexes described herein comprise one or more of the cationic lipids described in Formula I.

In another aspect, disclosed herein are compositions and methods that provide improved efficiency for introducing molecules and macromolecules, such as nucleic acids, proteins, peptides, nutrients and pharmaceuticals into cells. Accordingly, provided herein are compositions comprising a nucleic acid molecule, a transfection agent and a transfection enhancer.

In some embodiments, the transfection enhancer is a surface ligand that comprises amino acid sequences derived from a cell binding adhesion proteins. Collagen, fibronectin, lamin, veronectin, cadherin, nidogen, fibrinogen, elastin, bone asialoprotein, osteopontin and tenascin-C are non-limiting examples of cell binding adhesion proteins. In some embodiments, the surface ligand is the above-listed full-length protein. In other embodiments, the surface ligand is a fragment of the above-listed protein having greater than 5 amino acids in length. In other embodiments, the length of the fragment of the above-listed protein is greater than 5 amino acids is greater than 10 amino acids, greater than 15 amino acids, greater than 20 amino acids, greater than 25 amino acids, greater than 30 amino acids, greater than 35 amino acids, or greater than 40 amino acids.

In some embodiments, cell surface ligand, plant virus movement protein, or peptide derived therefrom proteins and describe herein comprise a nucleic acid binding moiety functionally linked to the amino acid sequence of the cell surface ligand, plant virus movement protein, or peptide derived therefrom proteins. Suitable nucleic acid binding moieties include, but are not limited to, a polycationic peptide sequence, a polyamine, a peptide nucleic acid, spermine, spermidine, carboxyspermidine, carboxy spermine, spermine and spermidine analogs, nucleic acid intercalaters, and the like. In certain embodiments, the nucleic acid binding moiety is covalently linked to the transfection promoting cell surface ligand comprising adhesion protein amino acid sequences. In further embodiments, the transfection agent is a cationic lipid, such as those described below, a polyamine, a polycationic peptide sequence, a cationic dendrimer, or the like.

In some embodiments, the cell surface ligand adhesion sequence or peptide derived from a plant movement protein is a multimer of itself or other adhesion sequences. In certain embodiments, the cell binding adhesion or peptide derived from a plant movement protein amino sequence is cyclized. In other embodiments, the surface ligands or peptide derived from a plant movement protein also contain other peptide sequence that enhance transfection efficiency, such as linkers, spacers, or nuclear targeting sequences.

In some embodiments, cell surface ligand, plant virus movement protein, or peptide derived therefrom described herein are attached directly to the binding molecule by covalent bonding, or are connected to the binding molecule via a spacer. The term “spacer,” or “linker,” which are used interchangeably herein, as used herein refers to a chemical structure that links two molecules to each other. In some embodiments, the spacer binds each molecule on a different part of the spacer molecule. In other embodiments, the spacer is a hydrophilic moiety and comprises about 6 to 30 carbon atoms. In other embodiments, the spacer comprises a ployether, for example —CH₂—O—(CH₂—CH₂—O—)ᵢCH₂—. In other embodiments, the spacer comprises a hydrophilic polymer, for example [(gly)ᵢ(ser)ⱼ]ₖ (SEQ ID NO: 585). In these formulae i ranges from 1 to 6, j ranges from 1 to 6, and k ranges from 3 to 20. In some embodiments, the spacer is a peptide of sequence APYKAWK (SEQ ID NO:505). In other embodiments, the spacer is a sequence that is degraded in vivo by a peptidase.

In some embodiments, the cell surface ligand, plant virus movement protein, or peptide derived therefrom described herein are functionally linked to a lipid, such as a cationic or neutral lipid. In some of these embodiments, the linked moiety is used for delivery of macromolecules into cells. For example, a cell surface ligand, plant virus movement protein, or peptide derived therefrom, or a cell binding adhesion peptide sequences amino acid sequence is covalently linked to a lipid, such as a cationic lipid, a lysolipid, using methods known in the art.

In certain embodiments, the cell surface ligand, plant virus movement protein, or peptide derived therefrom sequences described herein also are functionally linked to an amino acid sequence that inserts itself into lipid membranes, such as membrane anchor peptides or proteins. In other embodiments, the cell surface ligand peptide sequences are linked to chemical compositions that associate with lipids.

In other embodiments, the transfection complexes or liposomal compositions with the cell surface ligand, plant virus movement protein, or peptide derived therefrom described herein also comprise other transfection enhancing agents, such as a nuclear localization protein or peptide, a fusogenic peptide or protein, a transport peptide or protein, a viral peptide or protein, or a lysomoltropic agent. In certain embodiments, the viral peptide is derived from a virus enveloped or non-enveloped virus, for example an influenza virus, a vesicular stomatitis virus, an adenovirus, an alphavirus, a Semliki Forest Virus, a hepatitis virus, a herpes virus, an HIV virus, or a simian virus. In some embodiments, the transfection enhancing agent is, for example, insulin, a transferrin, a epidermal growth factor, a fibroblast growth factor, a cell targeting antibody or fragment from an antibody, a lactoferrin, a fibronectin, an adenovirus penton base, Knob, a hexon protein, a vesicular stomatitis virus glycoprotein, a Semliki Forest Virus core protein, an influenza hemagglutinin, a hepatitis B core protein, an HIV Tat protein, a herpes simplex virus VP22 protein, a histone protein, an arginine rich cell permeability protein, a high mobility group protein, and invasin protein, an internalin protein, an endotoxin, a diptheria toxin, a shigella toxin, a melittin, a magainin, a gramicidin, a cecrophin, a defensin, a protegrin, a tachyplesin, a thionin, a indolicidin, a bactenecin, a drosomycin, an apidaecin, a cathelicidin, a bacteriacidal-permability-increasing protein, a nisin, a buforin, or fragments thereof. In other embodiments, the transfection enhancing agent is chloroquine, a lysosomotrophic compound or combinations thereof. In other embodiments exosomes or exosomal derived lipids or proteins are the transfection enhanceagent. In other embodiments, the transfection enhancer agent comprises multimers of the same or different peptides or proteins.

Suitable nuclear localization peptides or proteins included in transfection complexes or liposomal compositions include, but are not limited to, a sequence selected from the group consisting of SEQ ID NOs:1-41, as set forth in Table 1, below, or in the sequence listings.

Proteins such as histones, protamines, HMG proteins, and viral core proteins or coat proteins comprise nuclear localization proteins. In some embodiments, these proteins or fragments thereof are used to enhance transfection. In some embodiments, the nuclear localization peptide is optionally linked to a nucleic acid binding moiety, for example via a covalent linkage. Spacer sequences are optionally used between the DNA binding sequence and the nuclear localization sequence. In some embodiments, the nuclear localization sequences are linked to helper lipids or other peptides proteins or compounds that associate with lipid bilayers.

In some embodiments, the compositions described herein also comprise a fusion agent or combinations of fusion agents, which in some embodiments also function as an amphipathic peptide. Suitable fusion peptides include, but are not limited to, a sequence selected from the group consisting of SEQ ID NOs:42-92, as set forth in Table 1, below, or in the sequence listings.

In some embodiments, the fusion agent is optionally linked to a nucleic acid binding moiety, for example via a covalent linkage. The peptides KK, KKK, KKKK (SEQ ID NO:97), RR, RRR, RRRR (SEQ ID NO:105) can be linked to fusion agents of SEQ ID NOs:42-92. In certain embodiments, fusion peptides are linked to helper lipids, cationic lipids, or other peptides or proteins that associate with lipid bilayers. Spacer sequences are optionally used between the DNA binding sequence and the fusion agent sequence

In certain embodiments, the compositions disclosed herein comprise a cell penetration agent or combinations of cell penetration agents. Suitable cell penetration agents include, but are not limited to, a sequence selected from the group consisting of SEQ ID NOs:93-96 as set forth in Table 1, below, or in the sequence listings.

In some embodiments, the cell penetration agents are optionally linked to a nucleic acid binding moiety, for example via a covalent linkage. In other embodiments, the cell penetration agents are linked to helper lipids or other peptides or proteins that associate with lipid bilayers.

In some embodiments, the nuclear localization sequences, the fusion agents, cell surface ligand or the cell penetration agents are linked to the GPI anchor peptides, the sequence FTLTGLLGTLVTMGLLT (SEQ ID NO:504) being a non limiting example.

In some embodiments, the nucleic acid binding moieties that are linked to different transfection enhancer and are part of transfection complexes have different binding affinity for nucleic acids depending on the needed functionality for attachment, condensation of nucleic acid, and the rate of release of nucleic acid from the nucleic acid binding moiety. Suitable nucleic acid binding moieties include, but are not limited to a polycationic peptide sequence, a polyamine, a peptide nucleic acid, spermine, spermidine, carboxyspermidine, carboxy spermine, spermine and spermidine analogs, nucleic acid intercalaters, and the like.

In some embodiments, the compositions described herein comprise combinations of different transfection enhancers with different nucleic acid binding moieties. Suitable nucleic acid binding peptides include, but are not limited to a sequence of SEQ ID NOs:97-149, as set forth in Table 1, below, or in the sequence listings.

In some embodiments, the nucleic acid binding moieties also serve as transfection enhancers when bound to nucleic acids, or alternatively serve as condensing agents. Suitable nucleic acid condensing peptides include, but are not limited to, a sequence selected from the group consisting of the peptides of SEQ ID NOs:97-149 as set forth in Table 1, below, or in the sequence listings. In some embodiments, multimers of these peptides are also synthesized and used as condensing agents. In some embodiments, nuclear localization sequences are also used as condensing agents if they have enough cationic charge.

Suitable nucleic acid binding moieties include, but are not limited to a polycationic peptide sequence, a polyamine, a peptide nucleic acid, spermine, spermidine, carboxyspermidine, carboxy spermine, spermine and spermidine analogs, nucleic acid intercalaters, and the like

One skilled in the art will readily recognize that the surface ligand chosen depends on which receptor is being bound. Since different types of cells have different receptors, this provides a method of targeting nucleic acid, peptides, protein, and compounds to specific cell types, depending on which cell surface ligand is used. Thus, the preferred cell surface ligand or ligands may depend on the targeted cell type.

In some embodiments, the transfection enhancers that are used in combination with the cell surface ligand, plant virus movement protein, or peptide derived therefrom disclose herein include, but are not limited to, the peptides or proteins selected from the group consisting of a collagen, a fibronectin, a lamin, a veronectin, a cadherin, a nidogen, a fibrinogen, a elastin, a bone asialoprotein, a osteopontin, a tenascin-C, Avadin, insulin, a transferrin, a epidermal growth factor, a fibroblast growth factor, a cell targeting antibody, a lactoferrin, an enveloped virus, a non-enveloped virus, an adenovirus penton base, a knob protein, a hexon protein, a vesicular stomatitis virus glycoprotein, a Semliki Forest Virus core protein, an influenza hemagglutinin, a hepatitis B core protein, an HIV Tat protein, a herpes simplex virus VP22 protein, a histone protein, an arginine rich cell permeability protein, a high mobility group protein, invasin protein, internalin protein, an endotoxin, a non-toxic diptheria toxin, a non-toxic shigella toxin, a melittin, a magainin, a gramicidin, a cecrophin, a defensin, a protegrin, a tachyplesin, a thionin, a indolicidin, a bactenecin, a drosomycin, an apidaecin, a cathelicidin, a bacteriacidal-permability-increasing protein, a nisin, a buforin, a fragment thereof, and a sequence selected from the group consisting of SEQ ID NOs:150-503, as set forth in Table 1, below, or in the sequence listings.

In some embodiments, the transfection enhancing agent is chloroquine, a lysosomotrophic compound or combinations thereof. In certain embodiments, the transfection enhancer agent comprises multimers of the same or different peptide enhancers, protein or protein fragments of transfection enhancers.

In some embodiments, the aforementioned peptides are optionally linked to a moiety selected from the group consisting of a nucleic acid binding moiety, a helper lipid, a cationic lipid, a cationic polymer, and a GPI anchor peptide.

In certain embodiments, the aforementioned peptides are optionally linked to a chemical moiety of Formula I,

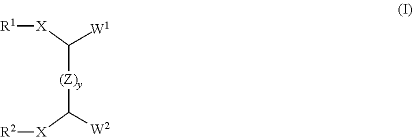

or a pharmaceutically acceptable salt thereof, where
W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, y=0; or
W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, y=0; or
W¹=W²=CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, y=0; or
W¹=W²=CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=0; or
W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, y=0; or
W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, y=0; or
W¹=W²=CH₂—N(R³R⁴), R¹=R²=C₈-C₂₂ alkyl, X=O; R³=CH₃, R⁴=—C(═O)-spermine, y=0; or
W¹=W²=CH₂—N(R³R⁴), R¹=R²=C₈-C₂₂ alkyl, X=O; R³=CH₃; R⁴=H, y=0; or
W¹=W²=CH₂—N(R³R⁴), R¹=R²=C₈-C₂₂ alkyl, X=O; R³=CH₃; R⁴=CH₂(CH₂)ₘ—NH₂; m=1-6, y=0; or
W¹=W²=CH₂—N(R³R⁴), R¹=R²=C₈-C₂₂ alkyl, X=O; R³=CH₃; R⁴=CH₂(CH₂)ₘ—NH—C(═O)-spermine; m=1-6, y=0; or
W¹=W²=CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, y=0; or
W¹=W²=CH₂—N(R³R⁴), R¹=R²=C₈-C₂₂ alkyl, X=O; R³=CH₃; R⁴=(CH₂)ₘ(CH—OH)(CH₂)ₘ—NH₂, y=0; or
W¹=W²=CH₂—N(R³R⁴), R¹=R²=C₈-C₂₂ alkyl, X=O; R³=CH₃; R⁴=CH₂(CH₂)ₙ—NH₂, y=0; or
W¹=H, W²=CH₂—N(R³R⁴)CH₂—R⁵; R³=R⁴=CH₃; R⁵=H, y=0; or
W¹=H; W²=CH₂—O—P(═O)(OMe)-O—CH₂CH₂—NH—C(═O)-spermine, y=0; or
W¹=H; W²=CH₂—O—P(═O)(OMe)-O—CH₂CH₂—NH—C(═O)-amino acid side chain, y=0; or
W¹=H; W²=CH₂—O—P(═O)(OMe)-O—CH₂CH₂—N⁺(CH₃)₃Cl, y=0; or
W¹=H; W²=CH₂—O—P(═O)(O⁻)—O—CH₂CH₂—NH—C(═O)-spermine, y=0; or
Z=(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, q=1-3, y=1; or
Z=(CH₂)_{q}, Wⁱ=W²=CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, q=1-3, y=1; or
Z=(CH₂)_{q}, W¹=W²=CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, q=1-3, y=1; or
Z=(CH₂)_{q}, W¹=W²=CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=1; or
Z=(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, q=1-3, y=1; or
Z=(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, q=1-3, y=1; or
Z=(CH₂)_{q} W¹=W²=CH₂—N(R³R⁴), R¹=R²=C₈-C₂₂ alkyl, X=O; R³=CH₃, R⁴=—C(═O)-spermine, q=1-3, y=1; or
Z=(CH₂)_{q}, W¹=W²=CH₂—N(R³R⁴), R¹=R²=C₈-C₂₂ alkyl, X=O; R³=CH₃; R⁴=H, q=1-3, y=1; or
Z=(CH₂)_{q} W¹=W²=CH₂—N(R³R⁴), R¹=R²=C₈-C₂₂ alkyl, X=O; R³=CH₃; R⁴=CH₂(CH₂)ₘ—NH₂; m=1-6, q=1-3, y=1; or
Z=(CH₂)_{q}, W¹=W²=CH₂—N(R³R⁴), R¹=R²=C₈-C₂₂ alkyl, X=O; R³=CH₃; R⁴=CH₂(CH₂)ₘ—NH—C(═O)-spermine; m=1-6, q=1-3, y=1; or
Z=(CH₂)_{q}, W¹=W²=CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, q=1-3, y=1; or
Z=(CH₂)_{q}, W¹=W²=CH₂—N(R³R⁴), R¹=R²=C₈-C₂₂ alkyl, X=O; R³=CH₃; R⁴=(CH₂)ₘ(CH—OH)(CH₂)ₘ—NH₂, q=1-3, y=1; or
Z=(CH₂)_{q}, W¹=W²=CH₂—N(R³R⁴), R¹=R²=C₈-C₂₂ alkyl, X=O; R³=CH₃; R⁴=CH₂(CH₂)ₙ—NH₂, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, q=1-3, y=1; or
Z=(CH₂)ₚ—N(R³)—(CH₂)ₚ, Wⁱ=W²=CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃, R⁴=—C(═O)-spermine, q=1-3, y=1; or
Z=(CH₂)_{q}—N(R³)—(CH₂)_{q}, Wⁱ=W²=CH₂—N(R³R⁴), R¹=R²=C₈-C₂₂ alkyl, X=O; R³=CH₃; R⁴=H, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃; R⁴=CH₂(CH₂)ₘ—NH₂; m=1-6, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃; R⁴=CH₂(CH₂)ₘ—NH—C(═O)-spermine; m=1-6, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃; R⁴=(CH₂)ₘ(CH—OH)(CH₂)ₘ—NH₂, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃; R⁴=CH₂(CH₂)ₙ—NH₂, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, q=1-3, y=1; or
Z=(CH₂)_{q}—S—S—(CH₂)_{q}, W¹=W²=CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃, R⁴=—C(═O)-spermine, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃; R⁴=H, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃; R⁴=CH₂(CH₂)ₘ—NH₂; m=1-6, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃; R⁴=CH₂(CH₂)ₘ—NH—C(═O)-spermine; m=1-6, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃; R⁴=(CH₂)ₘ(CH—OH)(CH₂)ₘ—NH₂, q=1-3, y=1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃; R⁴=CH₂(CH₂)ₙ—NH₂, q=1-3, y=1; or
Z=(CH₂)_{q} W¹=W²=CH₂—N(R³R⁴), R¹=R²=C₈-C₂₂ alkyl, X=O; R³=CH₃; R⁴=COCH₂(OCH₂CH₂)ₘ—NH₂; m=1-6, q=1-3, y=0 or 1; or
Z=(CH₂)_{q}—S—S—(CH₂)_{q}, Wⁱ=W²=CH₂—N(R³R⁴), R¹=R²=C₈-C₂₂ alkyl, X=O; R³=CH₃; R⁴=COCH₂(OCH₂CH₂)ₘ—NH₂; m=1-6, q=1-3, y=1; or
Z=(CH₂)_{q}—N(R³)—(CH₂)_{q}, W¹=W²=CH₂—N(R³R⁴), R¹=R²=C₈-C₂₂ alkyl, X=O; R³=CH₃; R⁴=COCH₂(OCH₂CH₂)ₘ—NH₂; m=1-6, q=1-3, y=1; or
W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃, R⁴=—C(═O)-spermine, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃; R⁴=H, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=CH₂—N(R³R⁴), R¹=R²=C₈-C₂₂ alkyl, X=O; R³=CH₃; R⁴=CH₂(CH₂)ₘ—NH₂; m=1-6, y=0; or
W¹=W²=—CH₂—B—CH₂—N(R³R⁴), R¹=R²=C₈-C₂₂ alkyl, X=O; R³=CH₃; R⁴=CH₂(CH₂)ₘ—NH—C(═O)-spermine; m=1-6, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃; R⁴=(CH₂)ₘ(CH—OH)(CH₂)ₘ—NH₂, y=0 where B is selected from the group —O(CH₂)ᵢ, —S(CH₂)ᵢ, —S—S(CH₂)ᵢ, —SO₂—(CH₂)ᵢ, i=1-5; or
W¹=W²=—CH₂—B—CH₂—N(R³R⁴), R¹=R²=C₈-C₂₂ alkyl, X=O; R³=CH₃; R⁴=CH₂(CH₂)ₙ—NH₂, y=0 where B is selected from the group —O(CH₂)ᵢ, —S(CH₂)ᵢ, —S—S(CH₂)ᵢ, —SO₂—(CH₂)ᵢ, i=1-5; or
W¹=H W²=—CH₂—B—CH₂—N(R³R⁴)CH₂—R⁵; R³=R⁴=CH₃; R⁵=H, y=0 where B is selected from the group —O(CH₂)ᵢ, —S(CH₂)ᵢ, —S—S(CH₂)ᵢ, —SO₂—(CH₂)ᵢ, i=1-5; or
W¹=H; W²=CH₂—O—P(═O)(OMe)-O—CH₂CH₂—NH—C(═O)-spermine, y=0; or
W¹=H; W²=CH₂—O—P(═O)(OMe)-O—CH₂CH₂—NH—C(═O)-amino acid side chain, y=0; or
W¹=H; W²=CH₂—O—P(═O)(OMe)-O—CH₂CH₂—N⁺(CH₃)₃Cl, y=0; or
W¹=H; W²=CH₂—O—P(═O)(O⁻)—O—CH₂CH₂—NH—C(═O)-spermine, y=0; or
Z=(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q} W¹=W²=—CH₂—B—CH₂—N(R³R⁴), R¹=R²=C₈-C₂₂ alkyl, X=O; R³=CH₃, R⁴=—C(═O)-spermine, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃; R⁴=H, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q} W¹=W²=—CH₂—B—CH₂—N(R³R⁴), R¹=R²=C₈-C₂₂ alkyl, X=O; R³=CH₃; R⁴=CH₂(CH₂)ₘ—NH₂; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH—C(═O)-spermine; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃; R⁴=(CH₂)ₘ(CH—OH)(CH₂)ₘ—NH₂, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₙ—NH₂, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)ₚ—N(R³)—(CH₂)ₚ, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃, R⁴=—C(═O)-spermine, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃; R⁴=H, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH₂; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH—C(═O)-spermine; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃; R⁴=(CH₂)ₘ(CH—OH)(CH₂)ₘ—NH₂, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₙ—NH₂, q=1-3, y=1 where B is selected from the group —O(CH₂)ᵢ, —S(CH₂)ᵢ, —S—S(CH₂)ᵢ, —SO₂—(CH₂)ᵢ, i=1-5;
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃, R⁴=—C(═O)-spermine, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃; R⁴=H, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH₂; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH—C(═O)-spermine; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃; R⁴=(CH₂)ₘ(CH—OH)(CH₂)ₘ—NH₂, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₙ—NH₂, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q} W¹=W²=—CH₂—B—CH₂—N(R³R⁴), R¹=R²=C₈-C₂₂ alkyl, X=O; R³=CH₃; R⁴=COCH₂(OCH₂CH₂)ₘ—NH₂; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—S—S—(CH₂)_{q}, W¹=W²=—CH₂—B—CH₂—N(R³R⁴), R¹=R²=C₈-C₂₂ alkyl, X=O; R³=CH₃; R⁴=COCH₂(OCH₂CH₂)ₘ—NH₂; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—N(R³)—(CH₂)_{q}, W¹=W²=—CH₂—B—CH₂—N(R³R⁴), R¹=R²=C₈-C₂₂ alkyl, X=O; R³=CH₃; R⁴=COCH₂(OCH₂CH₂)ₘ—NH₂; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=1; or
Z=(CH₂)_{q}—O—(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃, R⁴=—C(═O)-spermine, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃; R⁴=H, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃; R⁴=CH₂(CH₂)ₘ—NH₂; m=1-6, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃; R⁴=CH₂(CH₂)ₘ—NH—C(═O)-spermine; m=1-6, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃; R⁴=(CH₂)ₘ(CH—OH)(CH₂)ₘ—NH₂, q=1-3, y=1; or
Z=(CH₂)_{q}—O—(CH₂)_{q}, W¹=W²=CH₂—N(R³R⁴), R¹=R²=C₈-C₂₂ alkyl, X=O; R³=CH₃; R⁴=CH₂(CH₂)ₙ—NH₂, q=1-3, y=1; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—OH, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃, R⁴=—C(═O)—C(OH)—CH₂—(CH₂)ₙ—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH-iso-butyl)-CH₂—(CH₂)ₙ—O-iso-butyl, n=0-6, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃, R⁴=—C(═O)—C(NH₂)—CH₂—(CH₂)ₙ-His, n=0-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃, R⁴=—C(═O)-spermine, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃; R⁴=H, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH₂; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH—C(═O)-spermine; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5; or
Z=(CH₂)_{q}—O—(CH₂)_{q}, W¹=W²=—CH₂—B—CH₂—N(R³R⁴), R¹=R²=C₈-C₂₂ alkyl, X=O; R³=CH₃; R⁴=CH₂(CH₂)ₘ—NH—(C═O)-amino acid side chain; m=1-6, q=1-3, y=1 where B is selected from the group —O(CH₂)ᵢ, —S(CH₂)ᵢ, —S—S(CH₂)ᵢ, —SO₂—(CH₂)ᵢ, i=1-5.

In some embodiments, the compound of Formula I is a compound where:
R¹=R²=C₈-C₂₂ alkyl, X=O, W¹=H; W²=CH₂—O—P(═O)(OMe)-O—CH₂CH₂—NH—C(═O)-spermine, y=0;
W¹==CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, y=0;
W¹=W²=CH₂—N(R³R⁴), R²=C₈-C₂₂ alkyl, X=O; R³=CH₃, R⁴=—C(═O)-spermine, y=0;
W¹=W²=CH₂—N(R³R⁴), R²=C₈-C₂₂ alkyl, X=O; R³=CH₃, R⁴=—C(═O)—C(NH₂)—CH₂—(CH₂)ₙ—NH₂, n=0-6, y=0; or
W¹=W²=CH₂—N(R³R⁴), R²=C₈-C₂₂ alkyl, X=O; R³=CH₃; R⁴=(CH₂)ₘ(CH—OH)(CH₂)ₘ—NH₂, y=0.

In other embodiments, the compound of Formula I is a compound where:
R¹=R²=C₈-C₂₂ alkyl, X=O, W¹=H; W²=CH₂—O—P(═O)(OMe)-O—CH₂CH₂—NH—C(═O)-spermine, y=0; or
W¹==CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, y=0.

In other embodiments, the compound of Formula I is a compound where:
W¹=W²=CH₂—N(R³R⁴), R²=C₈-C₂₂ alkyl, X=O; R³=CH₃, R⁴=—C(═O)—C(NH₂)—CH₂—(CH₂)ₙ—NH₂, n=0-6, y=0; or
W¹=W²=CH₂—N(R³R⁴), R²=C₈-C₂₂ alkyl, X=O; R³=CH₃; R⁴=(CH₂)ₘ(CH—OH)(CH₂)ₘ—NH₂, y=0.

In other embodiments, the compound of Formula I is a compound where:
R¹=R²=C₈-C₂₂ alkyl, X=O, W¹=H; W²=CH₂—O—P(═O)(OMe)-O—CH₂CH₂—NH—C(═O)-spermine, y=0;
W¹==CH₂—N(R³R⁴), R¹=R²=C₈-C₂₂ alkyl, X=O; R³=CH₃, R⁴=—C(═O)—C(NH₂)—CH₂—(CH₂)ₙ—C(═NH)—NH₂, n=0-6, y=0; or
W¹=W²=CH₂—N(R³R⁴), R²=C₈-C₂₂ alkyl, X=O; R³=CH₃, R⁴=—C(═O)-spermine, y=0.

In some embodiments, the transfection agent comprises at least one or more cationic lipid from Formula I and may optionally also contain one or more neutral lipids; DOPE, DPhPE, saturated and unsaturated DPPE, saturated and unsaturated DMPE, cholesterol, DOPC, Lyso-PE (1-acyl-2-hydroxy-sn-glycero-3-phosphoethanolamine), Lyso-PC (1-acyl-3-hydroxy-sn-glycero-3-phosphocholine), 3-alkyloxy-2-hydroxy-1-acetamidopropane, 4-alkyloxy-3-hydroxy-1-acetamidopropane, 5-alkyloxy-4-hydroxy-1-acetamidopropane or 6-alkyloxy-5-hydroxy-1-acetamidopropane. In some embodiments, the alkyloxy in the above list is selected from the group consisting of myristyloxy, myristeleyloxy lauryloxy, palmityloxy, palmitoleyloxy, oleyloxy and streayloxy. In some embodiments transfection agents contains Lyso-phosphatidylcholine, Sphingomyelin, Disaturated phosphatidylcholine, saturated and unsaturated Phosphatidylcholine, Disaturated phosphatidylethanolamine, Phosphatidylethanolamine saturated and unsaturated, Phosphatidylserine, phosphatidylinositol, Lyso-bis phosphatidic acid, Cholesterol, and Diglyceride. Helper Lipids may include, complete exosomes solutions, total extract of lipids isolated from exosomes.

In other embodiment, the transfection agent comprises at least one or more cationic lipid of Formula I. In certain embodiments, the transfection agent optionally comprises one or more of cationic lipid, while in other embodiments, the agent optionally comprises one or more neutral lipids or one or more exosomes, or lipid extract for exosomes. In certain embodiments, the transfection agent comprises both one or more cationic lipid and one or more neutral lipid.

In some embodiments, the cationic lipid is selected from the group consisting of GeneIn™ (MTI-GlobalStem), TransfeX™ (ATCC), LipofectAmine™ 2000, LipofectAmine 3000, LipofectAmine™, Lipofectin®, DMRIE-C, CellFectin® (Invitrogen), Oligofectamine® (Invitrogen), LipofectAce® (Invitrogen), Fugene® (Promega), Fugene® HD (Promega), Transfectam® (Promega), Tfx-10® (Promega), Tfx-20® (Promega), Tfx-50® (Promega), DNA-In (MTI-GlobalStem), Transfectin™ (BioRad, Hercules, CA), SilentFect™ (Bio-Rad), Effectene® (Qiagen, Valencia, CA), DC-chol (Avanti Polar Lipids), GenePorter® (Gene Therapy Systems, San Diego, CA), DharmaFect 1® (Dharmacon, Lafayette, CO), DharmaFect 2® (Dharmacon), DharmaFect 3® (Dharmacon), DharmaFect 4® (Dharmacon), Escort™ III (Sigma, St. Louis, MO), Escort™ IV (Sigma), ViaFect™ (Promega), DOTMA, DOTAP, DMRIE, DC-Chol, DDAB, DOSPA, DOSPER, DOGS, TMTPS, TMTOS, TMTLS, TMTMS, TMDOS, N-1-dimethyl-N-1-(2,3-diaoleoyloxypropyl)-2-hydroxypropane-1,3-diamine, N-1-dimethyl-N-1-(2,3-diamyristyloxypropyl)-2-hydroxypropane-1,3-diamine, N-1-dimethyl-N-1-(2,3-diapalmityloxypropyl)-2-hydroxypropane-1,3-diamine, N-1-dimethyl-N-1-(2,3-diaoleoyl-oxypropyl)-2-(3-amino-2-hydroxypropyloxy)propane-1,3-diamine, N-1-dimethyl-N-1-(2,3-diamyristyloxypropyl)-2-(3-amino-2-hydroxypropyloxy)propane-1,3-diamine, N-1-dimethyl-N-1-(2,3-diapalmityloxypropyl)-2-(3-amino-2-hydroxypropyloxy)propane-1,3-diamine, L-spermine-5-carboxyl-3-(DL-1,2-dipalmitoyl-dimethylaminopropyl-β-hydr-oxyethylamine, 3,5-(N,N-di-lysyl)-diaminobenzoyl-glycyl-3-(DL-1,2-dipalmitoyl-di-methylaminopropyl-β-hydroxyethylamine), L-Lysine-bis(O,O′-oleoyl-β-hydroxyethyl)-amide dihydrochloride, L-Lysine-bis-(O,O′-palmitoyl-β-hydroxyethyl)amide dihydrochloride, 1,4-bis[(3-(3-aminopropyl)-alkylamino)-2-hydroxypropyl)piperazine, L-Lysine-bis-(O,O′-myristoyl-β-hydroxyethyl)amide dihydrochloride, L-Ornithine-bis-(O,O′-myristoyl-β-hydroxyethyl)amide dihydrochloride, L-Ornithine-bis-(O,O′-oleoyl-β-hydroxyethyl)amide dihydrochloride, 1,4-bis[(3-(3-aminopropyl)-oleylamino)-2-hydroxypropyl]piperazine, L-Ornithine-bis-(O,O′-palmitoyl-β-hydroxyethyl)amide dihydrochloride, 1,4,-bis[(3-amino-2-hydroxypropyl)-oleylamino]-butane-2,3-diol, 1,4,-bis[(3-amino-2-hydroxypropyl)-palmitylamino]-butane-2,3-diol, 1,4,-bis[(3-amino-2-hydroxypropyl)-myristylamino]-butane-2,3-diol, 1,4-bis[(3-oleylamino)propyl]piperaz-ine, L-Arginine-bis-(O,O′-oleoyl-β-hydroxyethyl)amide dihydrochloride, bis[(3-(3-aminopropyl)-myristylamino)2-hydroxypropyl]piperazine, L-Arginine-bis-(O,O′-palmitoyl-β-hydroxyethyl)amide dihydrochloride, L-Serine-bis-(O,O′-oleoyl-β-hydroxyethyl)amide dihydrochloride, 1,4-bis[(3-(3-aminopropyl)-palmitylamino)-2-hydroxypropyl]piperazine, Glycine-bis-(O,O′-palmitoyl-β-hydroxyethyl)amide dihydro-chloride, Sarcosine-bis-(O,O′-palmitoyl-β-hydroxyethyl)amide dihydrochloride, L-Histidine-bis-(O,O′-palmitoyl-β-hydroxyethyl)amide dihydrochloride, cholesteryl-3β-carboxyl-amidoethylenetrimethylammonium iodide, 1,4-bis[(3-myristylamino)propyl]-piperazine, 1-dimethylamino-3-trimethylammonio-DL-2-propyl-cholesteryl carboxylate iodide, cholesteryl-3β-carboxyamidoethyleneamine, cholesteryl-3β-oxysuccinamidoethyl-enetrimethylammonium iodide, 1-dimethylamino-3-trimethylammonio-DL-2-propyl-cholesteryl-3β-oxysuccinate iodide, 2-[(2-trimethylammonio)-ethylmethylamino] ethyl-cholesteryl-3β-oxysuccinate iodide, 3β[N—(N′,N′-dimethylaminoethane)carbamoyl]-cholesterol, and 3β-[N-(polyethyleneimine)-carbamoyl] cholesterol,1,4-bis[(3-palmitylamino)propyl]piperazine, L-Ornithylglycyl-N-(1-heptadecyloctadecyl)glycin-amide, N²,N⁵-Bis(3-aminopropyl)-L-ornithylglycyl-N-(1-heptadecyloctadecyl)glycin-amide, 1,4-bis[(3-(3-amino-2-hydroxypropyl)-alkylamino)-2-hydroxypropyl]piperazine N²—[N²,N⁵-Bis(3-aminopropyl)-L-ornithyl]-N,N-dioctadecyl-L-glutamine,N²—[N²,N⁵-Bis(aminopropyl)-L-ornithyl]-N—N-dioctadecyl-L-α-glutamine, 1,4-bis[(3-(3-amino-2-hydroxypropyl)-oleylamino)2-hydroxypropyl]piperazine, N²—[N²,N⁵-Bis(aminopropyl)-L-ornithyl]-N—N-dioctadecyl-L-α-asparagine, N—[N²—[N²,N⁵-Bis[(1,1-dimethylethoxy)-carbonyl]-N²,N⁵-bis[3-[(1,1-dimethylethoxy)carbonyl]aminopropyl]-L-ornithyl-N—N-dioctadecyl-L-glutaminyl]-L-glutamic acid, N²—[N²,N⁵-Bis(3-aminopropyl)-L-ornithyl]-N,N-diolyl-L-glutamine, N²—[N²,N⁵-Bis(aminopropyl)-L-ornithyl]-N—N-dioleyl-L-α-glutamine,4-bis[(3-(3-amino-2-hydoxypropyl)-myristylamino)-2-hydroxypropyl]piperaz-ine, N²—[N²,N⁵-Bis(aminopropyl)-L-ornithyl]-N—N-dioleyl-L-α-asparagine, N—[N²—[N²,N⁵-Bis[(1,1-dimethylethoxy)carbonyl]-N²,N⁵-bis[3-[(1,1-dimethylethoxy)carbonyl]-aminopropyl]-L-ornithyl-N—N-dioleyl-L-glutaminyl]-L-glutamic acid, 1,4-bis[(3-(3-aminopropyl)-oleylamino)propyl]piperazine, N²—[N²,N⁵-Bis(3-aminopropyl)-L-ornithyl]-N,N-dipalmityl-L-glutamine,N²—[N²,N⁵-Bis(aminopropyl)-L-ornithyl]-N—N-dipalmityl-L-α-glutamine, N²—[N²,N⁵-Bis(aminopropyl)-L-ornithyl]-N—N-dipalmityl-L-α-asparagine, N—[N²—[N²,N⁵-Bis[(1,1-dimethylethoxy)carbonyl]-N²,N⁵-bis[3-[(1,1-dimethylethoxy)-carbonyl]aminopropyl]-L-ornithyl-N—N-dipalmityl-L-glutaminyl]-L-glutamic acid, N²—[N²,N⁵-Bis(3-aminopropyl)-L-ornithyl]-N,N-dimyristyl-L-glutamine, N²—[N²,N⁵-Bis-(aminopropyl)-L-ornithyl]-N—N-dimyristyl-L-α-glutamine, N²—[N²,N⁵-Bis(aminopropyl)-L-ornithyl]-N—N-dimyristyl-L-α-asparagine, 1,4-bis[(3-(3-amino-2-hydroxypropyl)-palmitylamino)-2-hydroxypropyl]piperazine, N—[N²—[N²,N⁵-Bis[(1,1-dimethylethoxy)-carbonyl]-N²,N⁵-bis[3-[(1,1-dimethylethoxy)carbonyl]aminopropyl]-L-ornithyl-N—N-dimyristyl-L-glutaminyl]-L-glutamic acid, 1,4-bis[(3-(3-aminopropyl)-myristylamino)-propyl]piperazine, N²—[N²,N⁵-Bis(3-aminopropyl)-L-ornithyl]-N,N-dilaureyl-L-glutamine, N²—[N²,N⁵-Bis(aminopropyl)-L-ornithyl]-N—N-dilaureyl-L-α-glutamine, N²—[N²,N⁵-Bis(aminopropyl)-L-ornithyl]-N—N-dilaureyl-L-α-asparagine, N—[N²—[N²,N⁵-Bis[(1,1-dimethylethoxy)carbonyl]-N²,N⁵-bis[3-[(1,1-dimethylethoxy)carbonyl]amino-propyl]-L-ornithyl-N—N-dilaureyl-L-glutaminyl]-L-glutamic acid, 3-[N′,N″-bis(2-tert-butyloxycarbonylaminoethyl)guanidino]-N,N-dioctadec-9-enylpropionamide, 3-[N′,N″-bis(2-tertbutyloxycarbonylaminoethyl)guanidino]-N,N-dipalmitylpropionamide, 3-[N′,N″-bis(2-tertbutyloxycarbonylaminoethyl)guanidino]-N,N-dimyristylpropionamide, 1,4-bis[(3-(3-aminopropyl)-palmitylamino)propyl]piperazine, 1,4-bis[(3-(3-amino-2-hydroxypropyl)-oleylamino)propyl]piperazine, N,N-(2-hydroxy-3-aminopropyl)-N-2-hydroxypropyl-3-N,N-diolylaminopropane, N,N-(2-hydroxy-3-aminopropyl)-N-2-hydroxypropyl-3-N,N-dipalmitylaminopropane, N,N-(2-hydroxy-3-aminopropyl)-N-2-hydroxypropyl-3-N,N-dimyristylaminopropane, 1,4-bis[(3-(3-amino-2-hydoxypropyl)-myristylamino)propyl]piperazine, [(3-aminopropyl)-bis-(2-tetradecyloxyethyl)]methyl ammonium bromide, [(3-aminopropyl)-bis-(2-oleyloxyethyl)]methyl ammonium bromide, [(3-aminopropyl)-bis-(2-palmityloxyethyl)]methyl ammonium bromide, Oleoyl-2-hydroxy-3-N,N-dimethyamino propane, 2-didecanoyl-1-N,N-dimethylamino-propane, palmitoyl-2-hydroxy-3-N,N-dimethyamino propane, 1,2-dipalmitoyl-1-N,N-dimethylaminopropane, myristoyl-2-hydroxy-3-N,N-dimethyamino propane, 1,2-dimyristoyl-1-N,N-dimethylaminopropane, (3-Amino-propyl)->4-(3-amino-propyl-amino)-4-tetradecylcarbamoyl-butylcarbamic acid cholesteryl ester, (3-Amino-propyl)->4-(3-amino-propylamino-4-carbamoylbutylcarbamic acid cholesteryl ester, (3-Amino-propyl)->4-(3-amino-propylamino)-4-(2-dimethylamino-ethylcarbamoyl)-butylcarbamic acid cholesteryl ester, Spermine-5-carboxyglycine (N′-stearyl-N′-oleyl) amide tetratrifluoroacetic acid salt, Spermine-5-carboxyglycine (N′-stearyl-N′-elaidyl) amide tetratrifluoroacetic acid salt, Agmatinyl carboxycholesterol acetic acid salt, Spermine-5-carboxy-β-alanine cholesteryl ester tetratrifluoroacetic acid salt, 2,6-Diaminohexanoeyl β-alanine cholesteryl ester bistrifluoroacetic acid salt, 2,4-Diaminobutyroyl β-alanine cholesteryl ester bistrifluoroacetic acid salt, N,N-Bis (3-aminopropyl)-3-aminopropionyl β-alanine cholesteryl ester tristrifluoroacetic acid salt, [N,N-Bis(2-hydroxyethyl)-2-aminoethyl]aminocarboxy cholesteryl ester, Stearyl carnitine ester, Palmityl carnitine ester, Myristyl carnitine ester, Stearyl stearoyl carnitine ester chloride salt, L-Stearyl Stearoyl Carnitine Ester, Stearyl oleoyl carnitine ester chloride, Palmityl palmitoyl carnitine ester chloride, Myristyl myristoyl carnitine ester chloride, L-Myristyl myristoyl carnitine ester chloride, 1,4-bis[(3-(3-amino-2-hydroxypropyl)-palmitylamino)-propyl]piperazine, N-(3-aminopropyl)-N,N′-bis-(dodecyloxyethyl)-piperazinium bromide, N-(3-aminopropyl)-N,N′-bis-(oleyloxyethyl)-piperazinium bromide, N-(3-aminopropyl)-N,N′-bis-(palmityloxyethyl)-piperazinium bromide, N-(3-aminopropyl)-N,N′-bis-(myristyloxyethyl)-piperazinium bromide, N-(3-aminopropyl)-N′-methyl-N,N′-(bis-2-dodecyloxyethyl)-piperazinium bromide, N-(3-aminopropyl)-N′-methyl-N,N′-(bis-2-oleyloxyethyl)-piperazinium bromide, N-(3-aminopropyl)-N′-methyl-N,N′-(bis-2-palmityloxyethyl)-piperazinium bromide, N-(3-aminopropyl)-N′-methyl-N,N′-(bis-2-myristyloxyethyl)-piperazinium bromide, 1,4-bis[(3-(3-aminopropyl)-oleylamino)-2-hydroxy-propyl]piperazine, 1,4-bis[(3-(3-aminopropyl)-myristylamino)-2-hydroxy-propyl]piperazine, or 1,4-bis[(3-(3-aminopropyl)-palmitylamino)-2-hydroxy-propyl]piperazine, 3-alkyloxy-2-hydroxy-1-histidylamidopropane, 3-alkyloxy-2-hydroxy-1-aminopropane, 4-alkyloxy-3-hydroxy-1-histidylamidopropane, 4-alkyloxy-3-hydroxy-1-aminopropane, 5-alkyloxy-4-hydroxy-1-histidylamidopropane, 5-alkyloxy-4-hydroxy-1-aminopropane, 6-alkyloxy-5-hydroxy-1-histidylamidopropane, 6-alkyloxy-4-hydroxy-1-aminopropane; 2,3-dialkoxy-1,4-bis(N-methyl-N-carboxyspermineamido)-aminobutane, 2,3-dialkoxy-1,4-bis(N-methyl-N-histidinylamido)aminobutane, 2,3-dialkoxy-1,4-bis(N-methyl-N-arginylamido)aminobutane, 2,3-dialkoxy-1,4-bis(N-methyl-N-lysinylamido)aminobutane, 2,3-dialkoxy-1,4-bis(N-methyl-N′-ornithinyl-amido)aminobutane, 2,3-dialkoxy-1,4-bis(N-methyl-N-serinylamido)aminobutane, 2,3-dialkoxy-1,4-bis(N-methyl-N-homoerinylamido)aminobutane, 2,3-dialkoxy-1,4-bis(N-methyl-N-(diaminobutanyl)amido)aminobutane, 2,3-dialkoxy-1,4-bis(N-methyl-N-(di-aminopropyl)amido)aminobutane, 2,3-dialkoxy-1,4-bis(N-methyl-N-(2-hydroxylpropyl-amine))aminobutane, 2,3-dialkoxy-1,4-bis(N-methyl-N-(2-diaminopropyl))aminobutane, 2,3-dialkoxy-1,4- and bis(N-methyl-N-propylamine)aminobutane. The alkoxy in the above list may be myristyloxy, myristeleyloxy lauryloxy, palmityloxy, palmitoleyloxy, oleyloxy and streaylox.

In some embodiments transfection agents contains lyso-phosphatidylcholine, sphingomyelin, disaturated phosphatidylcholine, saturated and unsaturated phosphatidylcholine, disaturated phosphatidylethanolamine, phosphatidylethanolamine saturated and unsaturated, phosphatidylserine, phosphatidylinositol, lyso-bis phosphatidic acid, cholesterol, and diglyceride. Helper Lipids may include, complete exosomes solutions, total extract of lipids isolated from exosomes. The Helper lipids can be formulated into exosome like lipid particles so that lyso-phosphatidylcholine is from 3.75 to 6.21% of total lipid content, a sphingomyelin, a sphingosin, or ceramide is from 8.26 to 12, 41% of total, phosphatidylcholine-disaturated is from 3.00 to 4.81% of total, Phosphatidylethanolamine-mix is from 12.00 to 19.22%, phosphatidylserine is from 5.17 to 6.89%, phosphatidylinositol is from 5.17 to 6.89%, Phosphatidylethanolamine disaturated is from 2.61 to 2.85%, Phosphatidylethanolamine mix is from 13.42 to 14.65%, cholesterol is from 13.01 to 16.61%, diglycerides are from 4.76 to 7.05% and cationic lipids can be the remaining % in such formulations. Cationic lipids can be substituted or used in combination with a sphingomyelin, a sphingosin, or ceramides. Depending on cell type the % composition of helper lipids and can be adjusted to optimize formulations of synthetic exosomes with various transfection enhancers.

When the composition contains a neutral lipid, that lipid a saturated or unsaturated, or mixed acyl phospatidyl ethanol mine (PE) or phospahtidyl choline (PC), for example, DOPE, DPhPE, saturated and unsaturated DPPE, saturated and unsaturated DMPE, cholesterol, DOPC, Lyso-PE (1-acyl-2-hydroxy-sn-glycero-3-phosphoethanolamine), Lyso-PC (1-acyl-3-hydroxy-sn-glycero-3-phosphocholine), 3-alkyloxy-2-hydroxy-1-acetamidopropane, 4-alkyloxy-3-hydroxy-1-acetamidopropane, 5-alkyloxy-4-hydroxy-1-acetamidopropane or 6-alkyloxy-5-hydroxy-1-acetamidopropane. The alkyloxy in the above list may be myristyloxy, myristeleyloxy lauryloxy, palmityloxy, palmitoleyloxy, oleyloxy or streayloxy. In some embodiments, the tranfection agent. may contain more than one of these neutral lipids or exosomes, lipids from exosomes or total lipid extracts from exosomes compositions.

In other embodiments the transfection agent comprises at least one polyamine moiety. Suitable polyamines include dense star dendrimers, PAMAM dendrimers, NH₃ core dendrimers, ethylenediamine core dendrimers, dendrimers of generation 5 or higher, dendrimers with substituted groups, dendrimers having one or more amino acids, grafted dendrimers, activated dendrimers, polyethylenimine, and polyethylenimine conjugates, polycationic peptides such as polylysine, polyorinthine, polyhistidine, polyarginine

In other embodiments, cell surface ligand containing adhesion peptide sequences is conjugated to a nucleic acid binding group. In some of these embodiments, the nucleic acid binding group is linked to a polyamine or peptide nucleic acid. The polyamine optionally comprises at least one spermine moiety.

Suitable cell surface ligands containing adhesion peptide sequences that are derived from cell adhesion proteins include, but are not limited to, a sequence selected from the group consisting of SEQ ID NOs:202-503, as set forth in Table 1, below, or in the sequence listings.

In some embodiments, the peptides of SEQ ID NOs:202-503 are optionally linked to a nucleic acid binding moiety, a helper lipid, a cationic lipid, a cationic polymer, a GPI anchor peptide or other chemical moieties that associate with transfection complexes.

In some embodiments, the peptides of SEQ ID NOs:202-500 are used with other surface ligands, such as antibodies, antibody fragments, single chain antibodies, aptemers, or peptides from phage display. In certain embodiments, these surface ligands are optionally attached to nucleic acid binding moieties, to lipids or lipid associating moieties.

In particular embodiments, the transfection agent comprises at least one cationic lipid, and optionally also contains in various combinations with one or more neutral and/or helper lipids, targeting moieties, cell penetration agent, fusion agents, and lysomotropic agents.

In some embodiments, the presently disclosed complexes comprise one more agents selected from the group consisting of fusogenic agents, nuclear localization sequences, cell penetration agent, transport peptides, receptor-ligand or cell adhesion peptides.

It is to be understood that while some peptides are disclosed herein in the context of one particular use, all of the peptides presently disclosed can be used in other uses as well. Thus, by way of example only, the peptides of SEQ ID NOs:1-41 are disclosed to be nuclear localization peptides. However, these peptides can be used as a nucleic acid binding peptide, a nucleic acid condensing peptide, a transfection enhancer.

In specific embodiments, the cell surface ligand containing adhesion peptide sequences is covalently linked to the transfection agents, the cationic lipid, the neutral lipid, helper lipid, a chemical group that associates with lipids or liposomes, and/or the polyamine.

In specific embodiments, the plant virus movement protein or peptide derived from plant virus movement proteins is covalently linked to the transfection agents, the cationic lipid, the neutral lipid, helper lipid, a chemical group that associates with lipids or liposomes, and/or the polyamine.

In other embodiments, the plant virus movement protein or peptide derived from plant virus movement proteins is conjugated to a nucleic acid binding group. In some of these embodiments, the nucleic acid binding group is linked to a polyamine or peptide nucleic acid. The polyamine optionally comprises at least one spermine moiety.

Suitable virus movement proteins and peptides derived from plant virus movement proteins sequences include, but are not limited to, a sequence selected from the group consisting of SEQ ID NOs:506-580, as set forth in Table 1, below, or in the sequence listings.

In some embodiments, the peptides of SEQ ID NOs:506-580 are optionally linked to a nucleic acid binding moiety, a helper lipid, a cationic lipid, a cationic polymer, a GPI anchor peptide or other chemical moieties that associate with transfection complexes.

In some embodiments, the peptides of SEQ ID NOs:506-580 are used with other surface ligands, such as antibodies, antibody fragments, single chain antibodies, aptamers, or peptides from phage display. In certain embodiments, these surface ligands are optionally attached to nucleic acid binding moieties, to lipids or lipid associating moieties.

In particular embodiments, the transfection agent comprises at least one cationic lipid, and optionally also contains in various combinations with one or more neutral and/or helper lipids, targeting moieties, surface ligand, fusion agents, and lysomotropic agents.

In some embodiments, the presently disclosed complexes comprise one more agents selected from the group consisting of fusogenic agents, cell penetration agents, nuclear localization sequences, transport peptides, plant movement protein or peptide derived from, receptor-ligand, surface ligand or cell adhesion peptides.

It is to be understood that while some peptides are disclosed herein in the context of one particular use, all of the peptides presently disclosed can be used in other uses as well. Thus, by way of example only, the peptides of SEQ ID NOs:1-41 are disclosed to be nuclear localization peptides. However, these peptides can be used as a nucleic acid binding peptide, a nucleic acid condensing peptide, a transfection enhancer.

In another aspect, disclosed herein are pharmaceutical compositions containing a complex as described herein, and a pharmaceutically acceptable carrier.

In another aspect, disclosed herein are methods of transfecting a cell, by contacting a cell with a complex as described herein. In some embodiments, the cell is selected from the group consisting of a primary cell culture, a passaged cell culture, suspension cell line and an attached cell line. Suitable cells include all human cell lines and all animal cell lines. In some embodiments, the cell is a blood derived cell or the cell is a stem cell, while in other embodiments, the cell is a neuron.

In one method, a nucleic acid, protein or, peptide, or pharmaceutical is contacted with a cell surface ligand containing adhesion peptide, the plant virus movement protein, or peptide derived therefrom, capable associating with nucleic acid, protein, peptide or pharmaceutical and the resulting mixture is added to a transfection agent then contacted to cells.

In one embodiment of the transfection methods, cell surface ligand containing adhesion peptide, the plant virus movement protein, or peptide derived therefrom, are contacted with a transfection agent capable of associating with a nucleic acid, a protein, a peptide or a pharmaceutical composition, followed by addition of a nucleic acid, a protein, peptide or pharmaceutical then contacted to cells.

In another method, cell surface ligand containing adhesion peptide, the plant virus movement protein, or peptide derived therefrom, linked to a nucleic acid binding moiety is contacted with transfection agent capable of associating with nucleic acid, protein, peptide or pharmaceutical followed by addition of a nucleic acid, protein, peptide or pharmaceutical then contacted to cells

In another method, cell surface ligand containing adhesion peptide, the plant virus movement protein, or peptide derived therefrom, contacted with transfection agent followed by addition of a fusion agent and then contacted with a nucleic acid, protein, peptide or pharmaceutical then contacted to cells

In another method, cell surface ligand containing adhesion peptide, the plant virus movement protein, or peptide derived therefrom, linked to a nucleic acid binding moiety is contacted with transfection agent followed by addition of a fusion agent and then contacted with a nucleic acid, protein, peptide or pharmaceutical then contacted to cells

In another method cell surface ligand containing adhesion peptide, the plant virus movement protein, or peptide derived therefrom, is contacted with a fusion agent followed by addition of a transfection agent and then contacted with a nucleic acid, protein, peptide or pharmaceutical then contacted to cells.

In another method cell surface ligand containing adhesion peptide, the plant virus movement protein, or peptide derived therefrom, linked to a nucleic acid binding moiety is contacted with a fusion agent followed by addition of a transfection agent and then contacted with a nucleic acid, protein, peptide or pharmaceutical then contacted to cells.

In another method cell surface ligand containing adhesion peptide, the plant virus movement protein, or peptide derived therefrom, linked to a nucleic acid binding moiety is contacted with a transfection agent, then contacted with a nucleic acid, protein, peptide or pharmaceutical, then contacted with a fusion agent, and then contacted to cells.

In another method cell surface ligand containing adhesion peptide, the plant virus movement protein, or peptide derived therefrom, linked to a nucleic acid binding moiety is contacted with a transfection agent containing exosomes, or lipid extracts from exosomes, then contacted with a nucleic acid, protein, peptide or pharmaceutical, then contacted with a fusion agent, and then contacted to cells

In another method an aqueous solution of transfection enhancers, that may optionally contain one or more a plant virus movement proteins or peptides derived from plant virus movement proteins linked to a nucleic acid binding moiety, that may optionally contain buffers, that may optionally contain one or more cell surface ligands, that may optionally contain one or more nuclear localization agents, that may optionally contain one or more cell penetration peptides, that may optionally containing one or more fusion peptides or proteins, all or some of these transfection enhancers may optionally be linked to nucleic acid binding domain is mixed with an ethanol or aqueous solution of lipids that may contain cationic lipids, helper lipids, exosomes or total lipid extracts of exosomes or various mixtures of such lipids or lipid particles The mixture is then contacted with nucleic acids, protein, peptides, exosomes, growth enhancers, and then cells.

In another method an ethanol solution of transfection enhancers, that may optionally contain one or more a plant virus movement proteins or peptides derived from plant virus movement proteins linked to a nucleic acid binding moiety, that may optionally contain buffers, that may optionally contain one or more cell surface ligands, that may optionally contain one or more nuclear localization agents, that may optionally contain one or more cell penetration peptides, that may optionally containing one or more fusion peptides or proteins, all or some of these transfection enhancers may optionally be linked to nucleic acid binding domain is mixed with an ethanol or aqueous solution of lipids that may contain cationic lipids, helper lipids, exosomes or total lipid extracts of exosomes or various mixtures of such lipids or lipid particles The mixture is then contacted with nucleic acids, protein, peptides, exosomes, growth enhancers, and then cells.

In another method an aqueous solution of transfection enhancers, that may optionally contain one or more a plant virus movement proteins or peptides derived from plant virus movement proteins linked to a nucleic acid binding moiety, that may optionally contain buffers, that may optionally containing one or more cell surface ligands, that may optionally one or more nuclear localization agents, that may optionally contain one or more cell penetration peptides, all or some of these transfection enhancers may optionally be linked to nucleic acid binding domain is mixed with an ethanol or aqueous solution of lipids that may contain cationic lipids, helper lipids, exosomes or total lipid extracts of exosomes or various mixtures of such lipids or lipid particles. This mixture is then contacted with one or more fusion peptides or proteins which may optionally be in a buffer. The mixture is then contacted with nucleic acids, protein, peptides, exosomes, growth enhancers, and then cells.

In another method, a transfection complex is contacted with a nucleic acid, protein or, peptide then contacted with cell surface ligand containing adhesion peptide, the plant virus movement protein, or peptide derived therefrom, and then contacted to cells.

Those skilled in the art understand that concentrations of the various components, such as cationic lipid, helper lipid, the plant virus protein or peptide derived therefrom, cell surface ligand, fusogenic reagent, nuclear ligand, cationic polymer, condensing agent, cell penetration, lysomotrophic agent, exosomes or exosome lipids or proteins from exosomes, peptides derived for exosomal proteins and transfection enhancers are optimized according to the cell to be transfected or the in vivo application. In some embodiments, the concentration of each component ranges from 0.001 to 20 mg/mL, depending on solubility and formulation solvent.

In some embodiments, the plant virus movement protein derived peptides disclosed herein are suitable for use in the disclosed transfection enhancers to enhance transfection into HeLa cells, HuVec cells, iPS cells, NL-1 cells, C2C12 cells, human fibroblast cells, stem cells, Jurkat cells, rat cortical neurons, THP-1 cells, and human skeletal muscle cells, among others.

In some embodiments, the peptide useful to enhance the transfection of HeLa cells is selected from the group consisting of SEQ ID NOs:107, 205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, and 503 can be used in combination with the plant virus movement protein or peptide derived therefrom. In some of these embodiments, the peptide useful to enhance the transfection of HeLa cells is selected from the group consisting of SEQ ID NOs:205, 237, 268, 326, 328, 335, 336, 342, 347, 348, 352, 353, 355, 357, 358, 365, 367, 377, 379, 381, 450, 454, and 503 can be used in combination with the plant virus movement protein or peptide derived therefrom. In some embodiments, the peptide useful to enhance the transfection of HuVec cells is selected from the group consisting of SEQ ID NOs:205, 216, 218, 219, 220, 224, 226, 229, 230, 234, 236, 236, 237, 238, 239, 256, 268, 323, 326, 327, 328, 332, 335 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, and 503 can be used in combination with the cell surface ligand containing adhesion peptide, the plant virus movement protein, or peptide derived therefrom. In some of these embodiments, the peptide useful to enhance the transfection of HuVec cells is selected from the group consisting of SEQ ID NOs:236, 358, 373 can be used in combination with the cell surface ligand containing adhesion peptide, the plant virus movement protein, or peptide derived therefrom.

In some embodiments, the peptide useful to enhance the transfection of NL-1 iPS cells is selected from the group consisting of SEQ ID NOs:107, 205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, 503 can be used in combination with the cell surface ligand containing adhesion peptide, the plant virus movement protein, or peptide derived therefrom. In some of these embodiments, the peptide useful to enhance the transfection of NL-1 iPS cells is selected from the group consisting of SEQ ID NOs:216, 224, 226, 236, 236, 323, 327, 341, 343, 347, 348, 349, 350, 351, 354, 358, 360, 373, 383, 450, 454, 503 can be used in combination with the cell surface ligand containing adhesion peptide, the plant virus movement protein, or peptide derived therefrom.

In some embodiments, the peptide useful to enhance the transfection of C2C12 cells is selected from the group consisting of SEQ ID NOs:205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 350, 351, 352, 353, 354, 355, 357, 358, 359, 360, 361, 363, 365, 367, 369, 371, 373, 375, 377, 379, 381, 383, 450, 452, 454, 501, 502, 503 can be used in combination with the cell surface ligand containing adhesion peptide, the plant virus movement protein, or peptide derived therefrom. In some of these embodiments, the peptide useful to enhance the transfection of C2C12 cells is selected from the group consisting of SEQ ID NOs:218, 230, 237, 239, 256, 323, 326, 328, 335, 336, 342, 343, 345, 347, 348, 352, 357, 359, 367, 375, 379, 381, 450, 452, 454, 501, 502, 503 can be used in combination with the cell surface ligand containing adhesion peptide, the plant virus movement protein, or peptide derived therefrom,

In some embodiments, the peptide useful to enhance the transfection of human fibroblast cells is selected from the group consisting of SEQ ID NOs:205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, 501, 503 can be used in combination with cell surface ligand containing adhesion peptide, the plant virus movement protein, or peptide derived therefrom. In some of these embodiments, the peptide useful to enhance the transfection of human fibroblast cells is selected from the group consisting of SEQ ID NOs:205, 218, 219, 229, 230, 335, 336, 342, 344, 348, 349, 350, 351, 353, 355, 357, 361, 367, 369, 375, 379, 381, 450, 454, 501, 503 can be used in combination with the cell surface ligand containing adhesion peptide, the plant virus movement protein, or peptide derived therefrom.

In some embodiments, the peptide useful to enhance the transfection of Jurkat cells is selected from the group consisting of SEQ ID NOs:205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, 501, 503 can be used in combination with the cell surface ligand containing adhesion peptide, the plant virus movement protein, or peptide derived therefrom. In some of these embodiments, the peptide useful to enhance the transfection of Jurkat cells is selected from the group consisting of SEQ ID NOs:218, 349, 358 can be used in combination with the cell surface ligand containing adhesion peptide, the plant virus movement protein, or peptide derived therefrom.

In some embodiments, the peptide useful to enhance the transfection of rat cortical neuron cells is selected from the group consisting of SEQ ID NOs: 220, 236, 238, 323, 327, 336, 338, 341, 343, 347, 348, 350, 351, 352, 354, 367, 369, 373, 375, 377, 454 can be used in combination with the cell surface ligand containing adhesion peptide, the plant virus movement protein, or peptide derived therefrom.

In some embodiments, the peptide useful to enhance the transfection of THP-1 cells is selected from the group consisting of SEQ ID NOs:219, 229, 230, 239, 323, 328, 332, 341, 343, 350, 351, 357, 358, 375, 450, 454 can be used in combination with the cell surface ligand containing adhesion peptide, the plant virus movement protein, or peptide derived therefrom.

In some embodiments, the peptide useful to enhance the transfection of human skeletal muscle cells is selected from the group consisting of SEQ ID NOs: 218, 219, 230, 328, 336, 344, 350, 351, 353, 355, 365, 375 can be used in combination with the cell surface ligand containing adhesion peptide, the plant virus movement protein, or peptide derived therefrom.

In some embodiments combination of different surface ligands that have preference for a given cell types are mixed together to target multiple cell types in a single formulation.

In another aspect, disclosed herein are kits containing a transfection agent and a peptide or protein or a modified peptide or modified protein with cell surface ligand containing adhesion peptide, the plant virus movement protein, or peptide derived therefrom, as described herein. In some embodiments, the kit further comprises instructions for the preparation and the use of the transfection complexes. In certain embodiments, the kit further comprises separate compartments for the various components. In certain embodiments transfection agents may comprise various peptides selected from SEQ ID NOs 1-505 in combination with SEQ ID NOs 506-580 that are suitable for transfecting cells in vivo or in vitro.

### EXAMPLES

### Example 1: Surface Ligands

Cells were plated to so that on the day of transfection the cells were 70% confluent in 96 well tissue culture plates. A DOMTA/DOPE Lipid solution (1:1 molar ratio) at 2 mg/mL in water were mixed with an equal volume cell surface ligand containing adhesion peptide, the plant virus movement protein, or peptide derived therefrom, that had the nucleic acid binding moiety RRRRRRRRRRRR (SEQ ID NO:109) or KKKKKKKKKKKKKKKK (SEQ ID NO: 102) covalently linked to the N-terminus, or C-terminus of each peptide during peptide synthesis. Peptides were at 2 mg/mL in water. The DOTMA/DOPE and the solution containing the cell surface ligand containing adhesion peptide, the plant virus movement protein, or peptide derived therefrom, surface ligand peptide the was diluted 1 to 1 (v/v) in water. DOMTA/DOPE/PEPTIDE solutions were added to 0.1 mL of a plasmid DNA solution (EF1Alpha eGFP plasmid) at 10 μg/mL in OptiMEM.

All solutions were at room temperature. A volume of 0.2 mL of a 10 μg/mL solution of DNA was aliquoted into each well of a non-tissue cultured treated plate. 1-12 μL of transfection reagents were added to DNA solutions respectively. The transfection reagent and DNA solution was mixed by pipetting up and down twice. Transfection complexes were formed for 10 minutes. After 10 minutes, 0.01 or 0.02 mL of the transfection complex was added to cells. HuVEC, HeLa, human adult keratinocytes, human primary adult fibroblast, and A549 cells all respectively received 0.01 mL of the transfection complex. Human skeletal muscle cells, mouse C2C12 cells, and Jurkat, cells received 0.02 mL of the transfection complex. Cells were incubated for 42 hours at 37° C. at 5% CO₂. Plates were read on a fluorescent plate reader. Cells were also examined visually under a microscope to assess the extent of transfection (in terms of the percent of cells transfected) with a fluorescent microscope. Other modes of analysis, for example quantification with B-galactocidase or luciferase reports plasmids can also be used. If the plate reader did not show a sufficient signal to noise ratio, then plates were scored for cells transfected and those peptides that show increase over DOTMA alone were noted.

Not all surface ligands increased transfection efficiency even though they were suggested to be used to attach cell to tissue culture plates. Surface ligand that caused increase expression of GFP over the lipid DOTMA with no surface ligand were considered to enhance transfection or had higher % cells transfected were considered to enhance transfection. Peptides that gave greater than 2-fold enhancement are noted in bold and could be further optimized by adding different transfection enhancers or could be used with different cationic lipids or polymers.

Tables 2-7, below, provide the results of the transfection enhancements with the various cell types, while Table 8 lists the peptides that were determined by visual inspection to enhance the transfection of the denoted cell lines over the lipid DOTMA.

The following sequences increased transfection expression and the % cells transfected over the no peptide control on some examined cell types: SEQ ID NOs: 205, 216, 218, 219, 220, 226, 229,230, 236, 237, 238, 239, 256, 268, 323, 326, 327, 328, 332, 336, 338, 341, 342, 343, 344, 345, 347, 348, 349, 350, 351, 352, 353, 354, 355, 357, 358, 359, 360, 361, 365, 367, 369, 373, 375, 377, 379, 381, 393, 450, 452, 454, 501, and 503.

Various DOTMA/peptide transfection reagents that showed enhanced transfection efficiency over DOTMA alone were further formulated with the fusogenic peptide (SEQ ID NO:72). The fusogenic peptide was added to the DOMTA/peptide formulation to achieve a final concentration of 0.1 mg/mL to see if transfection reagents were further enhanced by the addition of a fusogenic peptide and could provide higher transfection efficiency into HeLa cells and expression than the commercially available Lipofectamine 2000. The results are shown in Table 9.

The following Table 1 lists the peptide sequences that are referenced herein.

TABLE 1

| SEQ | |
|---|---|
| ID | |
| NO | Sequence |
| 1 | GYSTPPKKKRKVEDP |
| 2 | GYSTPPKTRRRP |
| 3 | GYSTPGRKKR |
| 4 | GYSTPRRNRRRRW |
| 5 | PDEVKRKKKPPTSYG |
| 6 | PRRRTKPPTSYG |
| 7 | RKKRGPTSYG |
| 8 | WRRRRNRRPTSYG |
| 9 | GYGPPKKKRKVEAPYKA |
| 10 | PAAKRVKLD |
| 11 | RQRRNELKRSP |
| 12 | KRPAATKKAGQAKKKK |
| 13 | VRKKRKTEEESPLKDKDAKKSKQE |
| 14 | RLRRDAGGRGGVYEHLGGAPRRRK |
| 15 | KRKGDEVDGVDECAKKSKK |
| 16 | NQSSNFGPMKGGNFGGRSSGPYGGGGQYFAKPRNQGGY |
| 17 | GGKRTADGSEFESPKKARKVEAYPKAW |
| 18 | GGKRTADGSEFESPKKKRAVEAYPKAW |
| 19 | GGKRTADGSEFESPKKKAKVEAYPKAW |
| 20 | GGKRTADGSEFESPKKKRKVEAPYKAWK |
| 21 | GGKRTADGSEFESPKKKRKVEYKAWK |
| 22 | GYGPAAKRVKLDEAYPKAWK |
| 23 | GGKRTADGSEFEPAAKRVKLDEAYPKAWK |
| 24 | GTGPKKKRKVGGGGYGPKKKRLVG |
| 25 | KRPAATKKAGQAKKKKLEAYPKAWK |
| 26 | ATKGTKRSYEQMETGE |
| 27 | GKWERKPIRCAS |
| 28 | GYGKRTADSQHSTPPKKKRKVEAPYKAWK |
| 29 | KRTADSQHSTPPKKKRKVEAPYKAWK |
| 30 | GYGPPKKKRKVEAPYKAWKWAKYPAMRRAHHRRRRAS |
| | HRRRTTTGT |
| 31 | GYGPPKKKRKVEAPYKAWKRGARRYSKMKRRRRRVARRHRRRP |
| 32 | FWGYGYGPPKKKRKVEAPYKAWK |
| 33 | GKPSSDDEATADSQHSTPPKKKERKVED |
| 34 | GKPTADDQHSTPPKKKRKVED |
| 35 | GGKRTADGSEFESPKKARKVEAYPKAK |
| 36 | EKIRLRPGRKKRYRLKHL |
| 37 | PEGTRQARRNRRRRWRKR |
| 38 | PEGTRQPRRNRRRRWRKR |
| 39 | GVKRSYGAARGDDRRRPNVVAPYKAW |
| 40 | KSVPNRTRTYIKLKRLRFKGAPYKAW |
| 41 | EMRRRREEEGLQLRKQKREEQLFKRRN |
| 42 | FEAALAEALAEALA |
| 43 | Ac-LARLLPRLLARL-NHCH₃ |
| 44 | GLLEELLELLEELWEELLEG |
| 45 | GWEGLIEGIEGGWEGLIEG |
| 46 | GLFEALAEFIEGGWEGLIEG |
| 47 | GLFEALLELLESLWELLLEA |
| 48 | GGYCLEKWMIVASELKCFGNTA |
| 49 | GGYCLTRWMLIEAELKCFGNTAV |
| 50 | WEAALAEALAEALAEHLAEALAEALEALAA |
| 51 | GLFGAIAGFIENGWEGMIDGWYG |
| 52 | GIGAVLKVLTTGLPALISWIKRKRQQ |
| 53 | GRKKRRQRRRPPQ |
| 54 | RQIKIWFQNRRMKWKK |
| 55 | GWTLNSAGYLLGKINLKALAALAKKIL |
| 56 | WEAKLAKALAKALAKHLAKALAKALKACEA |
| 57 | GLFKALLKLLKSLWKLLLKA |
| 58 | GLFRALLRLLRSLWRLLLRA |
| 59 | GLFEALLELLESLYELLLEA |
| 60 | GLFEALEELWEA |
| 61 | GLFLLEEWLE |
| 62 | GLFLLEEWLEK |
| 63 | GLFEALLELLESLWELLLEAK |
| 64 | Suc-GLFKLLEEWLE |
| 65 | Suc-GLFKLLEEWLEK |
| 66 | GLFEAIAEFIEGGWEGLIEG |
| 67 | GLFKAIAKFIKGGWKGLIKG |
| 68 | IRFKKTKLIASIAMALC |
| 69 | ALAGTIIAGASLTFQVLDKVlEELGKVSRK |
| 70 | GLFEAIEGFIENGWEGMIDGWYG |
| 71 | GYICRRARGDNPDDRCT |
| 72 | GLFEAIAEFIEGGWEGLIEGCA |
| 73 | GLFHAIAHFIHGGWHGLIHGWWYG |
| 74 | RRRQRRKKRGGDIMGEWGNEIFGAIAGFLG |
| 75 | GLFEAIADFIENGWEGMIDGGG |
| 76 | ALAGTIIAGASLTFQVLDKVlEELGKVSRKK |
| 77 | IRFKKTKLIASTAMA |
| 78 | GLWHLLLHLWRRLLRLLR |
| 79 | KKIMLLLMTLLLVSLPLAQEQ |
| 80 | GLFEALLELLESLWELLLEAWYG |
| 81 | RLLRLLLRLWRRLLRLLR |
| 82 | LLELELLELELLLELELLELELLLEL |
| 83 | GLFEALLELLESLWELLLEARRRRRRRR |
| 84 | GLFEALLELLESLWELLLEARRRRRR |
| 85 | GLFEALLELLESLWELLLEAKKKKKKKK |
| 86 | GLFEALLELLESLWELLLEAKKKKKK |
| 87 | GLFEALLELLESLWELLLEAKK |
| 88 | GLFEALLELLESLWELLLEAKKKK |
| 89 | GLFEALLELLESLWELLLEAEE |
| 90 | GLFEALLELLESLWELLLEAEEEE |
| 91 | GLFEALLELLESLWELLLEAEEEEEE |
| 92 | GLFEALLELLESLWELLL |
| 93 | PLSSIFSRIGDPRGARRYAKMKRRRRRVARRHRRRP |
| 94 | GPFHYFQFLFPPV |
| 95 | GSSSWWQRWWPPW |
| 96 | RRRQRRKKR |
| 97 | KKKK |
| 98 | KKKKKK |
| 99 | KKKKKKKK |
| 100 | KKKKKKKKKK |
| 101 | KKKKKKKKKKKK |
| 102 | KKKKKKKKKKKKKKKK |
| 103 | KKKKKKKKKKKKKKKKKKKK |
| 104 | KKKKKKKKKKKKKKKKKKKKKKKK |
| 105 | RRRR |
| 106 | RRRRRR |
| 107 | RRRRRRRR |
| 108 | RRRRRRRRRR |
| 109 | RRRRRRRRRRRR |
| 110 | RRRRRRRRRRRRRRRR |
| 111 | RRRRRRRRRRRRRRRRRRRR |
| 112 | RRRRRRRRRRRRRRRRRRRRRRRR |
| 113 | YKA |
| 114 | KKKKKKKKWKGGGGACYGLPHLFCG |
| 115 | YKAKKKKKKKKWK |
| 116 | KTPKKAKKPKTPKKAKKP |
| 117 | KKAKKPAATRKSSKNPKKPKTVKPKKVAK |
| 118 | RGARRYSKMKRRRRRVARRHRRRP |
| 119 | TRQARRNRRRRWRERQRGSGSG |
| 120 | KRPRGRPKGSKKNWRRRKRRASRRSPRRR |
| 121 | KRGRGRPRKQPPKEPSEVPTPKRPRGRPKGSKNK |
| 122 | KEKYEKDIAAYRAKGKPAAKKGVVKAEKSKKKK |
| 123 | YKAKKKKKKKKKKWK |
| 124 | KKKKKKKGGC |
| 125 | YRARRRRRRRRWR |
| 126 | YRARRRRRRRRRRWR |
| 127 | KGDPKKPRGKMSSYAFFVQTCREEHKKKHPDASVNFSEFSKK |
| 128 | KKQLKKQLKKQLKQWK |
| 129 | KKSPKKSPKKSPKKSK |
| 130 | KLSKLEKKSKLEK |
| 131 | KLSKLEKKLSKLEKKSKLEK |
| 132 | KSLKKSLKKSLKKSK |
| 133 | KIRRRGKNKVAARTCRQRRTDR |
| 134 | KIRRRGKNKVAAQNCRKRKLET |
| 135 | KRRIRREKNKMAAAKCRNRRRELT |
| 136 | KDRSNLLERHTR |
| 137 | KRPAATKKAGQAKKKL |
| 138 | RRRRRREEEE |
| 139 | RRRRRREEEEEE |
| 140 | RRRRRREEEEEEEE |
| 141 | RRRRRRRREEEE |
| 142 | RRRRRRRREEEEEE |
| 143 | RRRRRRRREEEEEEEE |
| 144 | RRRRRRRRRRRREEEE |
| 145 | RRRRRRRRRRRREEEEEE |
| 146 | RRRRRRRRRRRREEEEEE |
| 147 | KLSKLEKK |
| 148 | SKLEK |
| 149 | KLSKLEKKLSKLEKK |
| 150 | PKKKRKVGGGRGDSP |
| 151 | LPHKSMPCG |
| 152 | GACLQHKSMPCG |
| 153 | YGLPHLFCG |
| 154 | SERSMNFCG |
| 155 | DHYSLYEDLERGTDK |
| 156 | ISLPRTSGAQRASTTR |
| 157 | EKLQTKYGLPHKVEFCG |
| 158 | TRISESQAKPGD |
| 159 | LVFFDY |
| 160 | WGGNGPTTFDCSGYTKYVFAK |
| 161 | INIGTTGWGDHYSLY |
| 162 | YDNIHG |
| 163 | AGWGKFLVGFGRV |
| 164 | SIGYPLP |
| 165 | TTHWGFTL |
| 166 | HLQIQPYPQISG |
| 167 | KLNIVSVNG |
| 168 | RGH |
| 169 | DNRIRLQAKAA |
| 170 | KIKMVISWKG |
| 171 | LPWYSYLYAVSA |
| 172 | WNLPWYYSVSPT |
| 173 | WNL |
| 174 | PWYYSVSPT |
| 175 | SSWESYKSGGGTRL |
| 176 | RDWSSQHPGRCNGETHLK |
| 177 | SLPTLTL |
| 178 | VICTGGDYSFALPVGQWPVMT |
| 179 | DKPSYQFGGHNSVDFEEDTLPKV |
| 180 | RARRRKRASATQLYQTCKASGTCPPD |
| 181 | SGDYSFALPVGQWPWMTG |
| 182 | CTGGDYSFALPVGQWPW |
| 183 | FYYDYDFFFDYWGQG |
| 184 | HLRRLRRRLLREAEG |
| 185 | DYYCAAWDDSLNGYSVF |
| 186 | YYCLQSMEDPYTFGG |
| 187 | YYCARSDGNYGYYYALDYDY |
| 188 | AARSPSYYRYDY |
| 189 | GPYYAMDYD |
| 190 | YYCQQRSSYPYTEGGAYPKAWK |
| 191 | YYCQRYDSDWSFGQGTKL |
| 192 | YYCARSGYYAMDYWGQGT |
| 193 | RVRRGACRGDCLG |
| 194 | RVRRGACRYDCLG |
| 195 | YYCAKGTHWGFWSGYFDYWGQGT |
| 196 | GRENYHGCTTHWGFTLC |
| 197 | VQATQSNQHTPRGGGSK |
| 198 | DPRAPGS |
| 199 | YYCQQRSSYPYTFGG |
| 200 | AARSPSYYRYDYGPYYAMDYD |
| 201 | GPKLTGILISILSLFVES |
| 202 | KYILRWRPKNS |
| 203 | IKVAV |
| 204 | WTPPRAQITGYRLTVGLTRR |
| 205 | AASIKVAVSADR |
| 206 | KLDAPT |
| 207 | NRWHSIYITRFG |
| 208 | PHSRN |
| 209 | SSFHFDGSGYAM |
| 210 | RGDS |
| 211 | IAFQRN |
| 212 | GRGDSP |
| 213 | TWYKIAFQRRK |
| 214 | EDGIHEL |
| 215 | SLVRNRRVITIQ |
| 216 | YRVRVTPKEKTGPMKE |
| 217 | LQVQLSR |
| 218 | SPPRRARVT |
| 219 | RKRLQVQLSIRT |
| 220 | ATETTITIS |
| 221 | NAPFPKLSWTIQ |
| 222 | VSPPRRARVTDATETTITISWRTKTETITGG |
| 223 | WTIQTTVDRGLL |
| 224 | KPDVRSYTITG |
| 225 | DTINNGRDHMILI |
| 226 | ANGQTPIQRYIK |
| 227 | MILISIGKSQKRM |
| 228 | PRARITGYIIKYEKPGSPPREVVPRPRPGV |
| 229 | PPFLMLLKGSTR |
| 230 | WQPPRARI |
| 231 | NQRLASFSNAQQS |
| 232 | WQPPRARITGYIIKYEKPG |
| 233 | ISNVFVQRMSQSPEVLD |
| 234 | YEKPGSPPREVVPRPRPGV |
| 235 | KARSFNVNQLLQD |
| 236 | KNNQKSEPLIGRKKT |
| 237 | KNSFMALYLSKG |
| 238 | EILDVPST |
| 239 | KNSFMALYLSKGRLVFALG |
| 240 | IDAPS |
| 241 | RDSFVALYLSEGHVIFAGLG |
| 242 | VVIDASTAIDAPSNL |
| 243 | KPRLQFSLDIQT |
| 244 | LDVPS |
| 245 | DGQWHSVTVSIK |
| 246 | REDV |
| 247 | FVLYLGSKNAKK |
| 248 | PHSRNRGDSP |
| 249 | LAIKNDNLVYVY |
| 250 | LWVTVRSQQRGLF |
| 251 | AYFSIVKIERVG |
| 252 | GINNWWQSPSIQN |
| 253 | DVISLYNFKHIY |
| 254 | WVTVTLDLRQVFQ |
| 255 | FFDGSSYAVVRD |
| 256 | RQVFQVAYIIIKA |
| 257 | LHVFYDFGFGFSNG |
| 258 | LTRYKITPRRGPPT |
| 259 | LKKAQINDAKYREISIIYHN |
| 260 | LLEFTSARYIRL |
| 261 | RAYFNGQSFIAS |
| 262 | YIRLRLQRIRTL |
| 263 | SRLRGKNPTKGK |
| 264 | RRYYYSIKDISV |
| 265 | LHKKGKNSSKPK |
| 266 | SINNTAVNQRLT |
| 267 | RLKTRSSHGMIF |
| 268 | GGFLKYTVSYDI |
| 269 | GEKSQFSIRLKT |
| 270 | RDQLMTVLANVT |
| 271 | TLFLAHGRLVFM |
| 272 | ANVTHLLIRANY |
| 273 | LVFMFNVGHKKL |
| 274 | AGTFALRGDNPQG |
| 275 | TLFLAHGRLVFMFNVGHKKL |
| 276 | VLIKGGRARKHV |
| 277 | DFMTLFLAHGRLVFMGNVG |
| 278 | LSNIDYLIKAS |
| 279 | HKKLKIRSQEKY |
| 280 | LQQSRIANISME |
| 281 | GAAWKIKGPIYL |
| 282 | NLLLLLVKANLK |
| 283 | VIRDSNVVQLDV |
| 284 | HRDELLLWARKI |
| 285 | GLIYYVAHQNQM |
| 286 | KRRARDLVHRAE |
| 287 | DYATLQLQEGRLHFMFDLG |
| 288 | SQFQESVDNITK |
| 289 | KKGSYNNIVVHV |
| 290 | PGGMREKGRKAR |
| 291 | ADNLLFYLGSAK |
| 292 | MEMQANLLLDRL |
| 293 | GSAKFIDFLAIE |
| 294 | LSEIKLLISAR |
| 295 | KVSFLWWVGSGV |
| 296 | RDFTKATNIRLRFLR |
| 297 | SYWYRIEASRTG |
| 298 | ISTVMFKFRTFS |
| 299 | YFDGTGFAKAVG |
| 300 | KQANISIVDIDSN |
| 301 | NGQWHKVTAKKI |
| 302 | FSTRNESGIILL |
| 303 | AKKIKNRLELVV |
| 304 | RRQTTQAYYAIF |
| 305 | GFPGGLNQFGLTTN |
| 306 | YAIFLNKGRLEV |
| 307 | NQFGLTTNIRFRG |
| 308 | KNRLTIELEVRT |
| 309 | IRSLKLTKGTGKP |
| 310 | GLLFYMARINHA |
| 311 | AKALELRGVQPVS |
| 312 | VQLRNGFPYFSY |
| 313 | GQLFHVAYILIKF |
| 314 | HKIKIVRVKQEG |
| 315 | NVLSLYNFKTTF |
| 316 | DFGTVQLRNGFPFFSYDLG |
| 317 | SQRIYQFAKLNYT |
| 318 | NIRLRFLRTNTL |
| 319 | EVNVTLDLGQVFH |
| 320 | GKNTGDHFVLYM |
| 321 | GQVFHVAYVLIKF |
| 322 | VVSLYNFEQTFML |
| 323 | HQQDLGTAGSCLRKFSTMFLF |
| 324 | RFDQELRLVSYN |
| 325 | HQQDLGTAGSCLRKFSTMFLFCNI |
| 326 | RLVSYSGVLFFLK |
| 327 | VAEIDGIEL |
| 328 | NWRHISYITRFG |
| 329 | GIIFFL |
| 330 | KRLQVQLRSIRT |
| 331 | ASKAIQVFLLGG |
| 332 | TWYKIAFQRNRK |
| 333 | VLVRVERATVFS |
| 334 | QVFQVAYIIIKA |
| 335 | TVFSVDQDNMLE |
| 336 | GEFYFDLRLKGDK |
| 337 | RLRGPQRVFDLH |
| 338 | GTPGPQGIA |
| 339 | FDLHQNMGSVN |
| 340 | GQRDVV |
| 341 | LRAHAVDVNG |
| 342 | TAGSCLRKFSTM |
| 343 | LFSHAVSSNG |
| 344 | KGHRGF |
| 345 | TAGSCLRKFSTMFLF |
| 346 | TAGSCLRKFSTMFLFCNI |
| 347 | DLGTAGSCLRKFSTM |
| 348 | HQQDLGTAGSCLRKFSTM |
| 349 | RNIAEIIKDI |
| 350 | SIGFRGDGQTC |
| 351 | LNRQELFPFG |
| 352 | RIQNLLKITNLRIKFVK |
| 353 | KKQRFRHRNRKGYRSQ |
| 354 | SINNTAVMQRLT |
| 355 | FRHRNRKGY |
| 356 | RYRVRVTPKEKTGPMKE |
| 357 | SETTVKYIFRLHE |
| 358 | GHRGPTGRPGKRGKQGQKGDS |
| 359 | KAFDITYVRLKF |
| 360 | GDLGRPGRKGRPGPP |
| 361 | YIGSR |
| 362 | RGEFYFDLRLKGDK |
| 363 | LAGSCLARFSTM |
| 364 | LALFLSNGHFVA |
| 365 | ISRCQVCMKKRH |
| 366 | PGRWHKVSVRWE |
| 367 | TDIPPCPHGWISLWK |
| 368 | VRWGMQQIQLVV |
| 369 | TAIPSCPEGTVPLYS |
| 370 | KMPYVSLELEMR |
| 371 | GPAGKDGEAGAQG |
| 372 | VLLQANDGAGEF |
| 373 | GLPGER |
| 374 | DGRWHRVAVIMG |
| 375 | LAGSCLPVFSTL |
| 376 | APVNVTASVQIQ |
| 377 | TAGSCLRRFSTM |
| 378 | KQGKALTQRHAK |
| 379 | TAGSCLRKF |
| 380 | RYVVLPR |
| 381 | TAGSCL |
| 382 | SPYTFIDSLVLMPY |
| 383 | TAG |
| 384 | PDSGR |
| 385 | QQNLGSVNVSTG |
| 386 | SRATAQKVSRRS |
| 387 | DPGYIGSR |
| 388 | GSLSSHLEFVGI |
| 389 | VILQQSAADIAR |
| 390 | RNRLHLSMLVRP |
| 391 | KDISEKVAVYST |
| 392 | APMSGRSPSLVLK |
| 393 | LGTIPG |
| 394 | AFGVLALWGTRV |
| 395 | TDIRVTLNRLNTF |
| 396 | IENVVTTFAPNR |
| 397 | AFSTLEGRPSAY |
| 398 | LEAEFHFTHLIM |
| 399 | TSAEAYNLLLRT |
| 400 | HLIMTFKTFRPA |
| 401 | LNRRYEQARNIS |
| 402 | KTWGVYRYFAYD |
| 403 | SLLSQLNNLLDQ |
| 404 | TNLRIKFVKLHT |
| 405 | RDIAEIIKDI |
| 406 | KRLVTGQR |
| 407 | SHAVSS |
| 408 | GPGVVVVERQYI |
| 409 | ADTPPV |
| 410 | NEPKVLKSYYYAI |
| 411 | LRAHAVDING |
| 412 | YYAISDFAVGGR |
| 413 | DSITKYFQMSLE |
| 414 | LPFFNDRPWRRAT |
| 415 | YTALIIATDN |
| 416 | FDPELYRSTGHGGH |
| 417 | VITVKDINDN |
| 418 | TNAVGYSVYDIS |
| 419 | GLDRESYPYY |
| 420 | APVKFLGNQVLSY |
| 421 | MKVSATDADD |
| 422 | SFSFRVDRRDTR |
| 423 | PQVTRGDVFTMP |
| 424 | TWSKVGGHLRPGIVQSG |
| 425 | KEAEREVTDLLR |
| 426 | RGDV |
| 427 | AAEPLKNIGILF |
| 428 | FALWDAIIGEL |
| 429 | VGVAPG |
| 430 | LWPLLAVLAAVA |
| 431 | PGVGV |
| 432 | VFDNFVLK |
| 433 | TSIKIRGTYSER |
| 434 | TTSWSQCSKS |
| 435 | DPETGV |
| 436 | KRSR |
| 437 | QGADTPPVGV |
| 438 | SVVYGLR |
| 439 | PLDREAIAKY |
| 440 | DGRGDSVAYG |
| 441 | HAVDI |
| 442 | LALERKDHSG |
| 443 | DQNDN |
| 444 | YSMKKTTMKIIPFNRLTIG |
| 445 | QDPELPDKNM |
| 446 | RGDF |
| 447 | LVVQAADLQG |
| 448 | GVYYQGGTYSKAS |
| 449 | NDDGGQFVVT |
| 450 | TAGSCLRKFSCL |
| 451 | YILHVAVTN |
| 452 | CNYYSNSYSFWLASLNPER |
| 453 | TYRIWRDTAN |
| 454 | TGLSCLQRFTTM |
| 455 | GFTCECSIGFRGDGQTCYGIVFWSEV |
| 456 | HHLGGAKQAGDV |
| 457 | SCLPGFSGDGRACRDVDECGH |
| 458 | MAPRPSLAKKQRFRHRNRKGYRSQRGHSRG |
| 459 | KKQKFRHRNRKGYRSQ |
| 460 | KKQKFKHRNRKGYRS |
| 461 | KKQKFRRRNRKGYRSH |
| 462 | TAIPPCPHGWISLWK |
| 463 | KKQKSRHRSRKRYRS |
| 464 | KKQKSRRRSRKGYRS |
| 465 | ISRCTVC |
| 466 | ISRCQVCMKRRH |
| 467 | VSRCTVC |
| 468 | TDIPPCPQGWISLWK |
| 469 | TVKAGELEKIISRCQVMKKRH |
| 470 | TDIPSCPHGWISLWK |
| 471 | TDIPPCPAGWISLWK |
| 472 | TEIPPCPQGWISLWK |
| 473 | TDVPPCPQGWISLWK |
| 474 | RLVSYNGILFFLK |
| 475 | RLVSYSGVIFFLK |
| 476 | RLVSYNGILFFL |
| 477 | RLVSYSGIIFFLK |
| 478 | RFEQELRLVSYSGVLFFLKQ |
| 479 | RLVSYNGIIFFLK |
| 480 | DPAFKIEDPYSPRIQNLLKITNLRIKFVKL |
| 481 | TKRFEQELRLVSYSGVLFFL |
| 482 | GGRLKYSVAF |
| 483 | GGFLRYTVSYDI |
| 484 | GGFLKYTVSYDV |
| 485 | LGNKLTAFGGFLKYTVSYDIPV |
| 486 | GGYLKYTVSYDI |
| 487 | GEIFFDMRLKGDK |
| 488 | GEIYFDLRLKGDK |
| 489 | GEIYLDMRLKGDK |
| 490 | IGQPGAKGEPGEFYFDLRLKGDKGDPGFPG |
| 491 | GEVFFDMRLKGDK |
| 492 | LAGSCLPIFSTL |
| 493 | AHNQDLGLAGSCLARFSTMPFLYCNPGDIC |
| 494 | QEKAHNQDLGLAGSCLPVFSTLPFAYCNIH |
| 495 | LAGSCLPVFSTM |
| 496 | GNKRAHGQDLGTAGSCLRRFSTMPFNIFCNI |
| 497 | RAHGQDLGTAGSCLRRFSTMP |
| 498 | RKRLQVQLNIRT |
| 499 | HLVLPLQQSDVRKRLQVQLSIRTFASSGLI |
| 500 | RKRLSVQLRIRT |
| 501 | DLGTAGSCLRRFSTM |
| 502 | RNIAEIIKDI |
| 503 | TAGSCLRKFSTMRRRRRRRRRRRR |
| 504 | FTLTGLLGTLVTMGLLT |
| 505 | APYKAWK |
| 506 | STSKTNRGDDSNWSKRVTNNKPS |
| 507 | STSKRKRGDDSNWSKRVTKKKPS |
| 508 | STSKRKRGDDSNWSKRVSKKKPS |
| 509 | STSKRKRGDDANWSKRVTKKKPS |
| 510 | PLAGSKRKRADEVAWSKRGTKKKPER |
| 511 | PLAGSKRKRADEVAWSKRGTKKKPERTSAARAGPSRRIR |
| 512 | STSKRKRGDDANWSKRTTKKKPSS |
| 513 | STSKRKRGDDANWSKRTTKKKPSSAGLKRAGSKADRPSL |
| 514 | PTTAGKRKRSDDAAWSKRARPKAGRT |
| 515 | PTTAGKRKRSDDAAWSKRARPKAGRTSAARPGTSVRRIR |
| 516 | SSSLGKRKRSDEGAWSKGKSKKKAMR |
| 517 | SSSLGKRKRSDEGAWSKGKSKKKAMRGSSSRRPGPVRGP |
| 518 | PTTAGKRKRTDDAAWSKRARPKAGR |
| 519 | PTTAGKRKRTDDAAWSKRARPKAGRTSAARPGTAVRRVR |
| 520 | PATAGKRKRSDDAAWSKRARPKAGRTSAAR |
| 521 | PATAGKRKRSDDAAWSKRARPKAGRTSAARPGTSVRRIR |
| 522 | SSSLGKRKRSNGGDWSKRSAKKKPA |
| 523 | SSSLGKRKRSNGGDWSKRSAKKKPAGTPSRRAGPGRGPR |
| 524 | SSSLGKRKRSDEGAWSKGKSKKKAMR |
| 525 | SSSLGKRKRSDEGAWSKGKSKKKAMRGSSSRRPGPVRGP |
| 526 | STSKRKRGDDANWNKRPTKKKPSS |
| 527 | STSKRKRGDDANWNKRPTKKKPSSAGLKKAGSKAERPSL |
| 528 | SGALKRKRSDEVAWSRRRPVKKPV |
| 529 | SGALKRKRSDEVAWSRRRPVKKPVRRAPPPRAGPSVRRG |
| 530 | SGALKRKRSDEVAWSRRKPAKKPAR |
| 531 | SGALKRKRSDEVAWSRRKPAKKPARQPPPPRAGPSVRRG |
| 532 | AGALKRKRSDEVAWSRRKPAKKPAR |
| 533 | AGALKRKRSDEVAWSRRKPAKKPARAPPPRAGPSVRRGL |
| 534 | STSKRKRGDDSNWSKRVTKKKPSSAGLKRAGSKADRPSL |
| | QIQTLQHAGTTMITVPSGGVCDLINTYARGSDEGNRHTS |
| | ETLTYKIAIDYHFVADAAACRYSNTGTGVMWLVYDTTPG |
| | GQAPTPQTIFSYPDTLKAWPATWKVSRELCHRFVVKRRW |
| | LFNMETDGRIGSDIPPSNASWKPCKRNIYFHKFTSGLGV |
| | RTQWKNVTDGGVGAIQRGALYMVIAPGNGLTFTAHGQTR |
| | LYFKSVGNQ |
| 535 | DPQNALYYQPRVPTAAPTSGGVPWSRVGEVAILSFVALI |
| | CFYLLYLWVLRDLILVLKARQGRSTEELIFGGQAVDRSN |
| | PIPNIPAPPSQGNPGPFVPGTG |
| 536 | GSQLVPPPSAFNYIESQRDEFQLSHDLTEIVLQFPSTAS |
| | QITARLSRSCMKIDHCVIEYRQQVPINASGTVIVEIHDK |
| | RMTDNESLQASWTFPIRCNIDLHYFSSSFFSLKDPIPWK |
| | LYYRVSDSNVHQMTHFAKFKGKLKLSSAKHSVDIPFRAP |
| | TVKILAKQFSEKDIDFWHVGYGKWERRLVKSASSSRFGL |
| | RGPIEINPGESWATKSAIVTPNRNADLDIEEELLPYREL |
| | NRLGTNILDPGESASIVGIQRSQSNITMSMSQLNELVRS |
| | TVHECIKTSCIPSTPKSLS |
| 537 | RTGVKRSYGAARGDDRRRPNVV |
| 538 | SYVKTVPNRTRTYIKLRVR |
| 539 | MYSTSNRRGRSQTQRGSHVRRTGVKRSYGAARGDDRRRP |
| | NVVSKTQVEPRMTIQRVQENQFGPEFVLSQNSALSTFVT |
| | YPSYVKTVPNRTRTYIKLKRVRFKGTLKIERGQGDTIMD |
| | GPSSNIEGVFSMVIVVDRKPHVSQSGRLHTFDELFGARI |
| | HCHGNLSVVPALKDRYYIRHVTKRVVSLEKDTLLIDLHG |
| | TTQLSNKRYNCWASFSDLERDCNGVYGNITKNALLVYYC |
| | WLSDAQSKASTYVSFELDYLG |
| 540 | R₁₂-VDYGKWERKPIRCASMSR |
| 541 | R₁₂-GKWERKPIRCAS |
| 542 | K₁₆-GKWERKPIRCAS |
| 543 | R₁₂-VDFSHVDYGKWERKPIRCASMSRLGLRG |
| 544 | GVKRSYGAARGDDRRRPNVVAPYKAW-R₁₂ |
| 545 | KSVPNRTRTYIKLKRLRFKGAPYKAW-R₁₂ |
| 546 | RTGVKRSYGAARGDDRRRPNVV-R₁₂ |
| 547 | SYVKTVPNRTRTYIKGGGGG-R₁₂ |
| 548 | VDIPFRAPTIKILSKQFTEDDIDFWHVGYGKWERKLVRPA |
| | SLSGRRGLRR |
| 549 | IDFWHVGYGKWERKLVRPASLSGRRGLRR |
| 550 | IDFWSVEKGETRRRLLNPTPHAHSPRPIAHR |
| 551 | IDFSHVGYGKWERKMIRSASISRLGLHN |
| 552 | VDFSHVGYGKWERKLIRSASTVKYGLPS |
| 553 | IDFSHVDYGKVERKLVKCESSSRLGLHS |
| 554 | IDFWSVGRKAQQRKLVQGPSLIGSRSMRY |
| 555 | IDFWSVGSKPQTRRLVDGSRLIGHSSRSLRV |
| 556 | IDFWSVERGETRRRLLNPTPSAGSNRALSKR |
| 557 | VDFWSVGKPKPIRRLIQNDPGTDYDTGPKYR |
| 558 | VDFWSVEKPKPIRRLLNPGPNQGPYPNTGHR |
| 559 | VDFSHVDYGKWERKLIRSASTSRYGLRS |
| 560 | VDFSHVDYGKWERKTLRSRSLSRIGLTG |
| 561 | IDFWHVGYGKWERRLVKSASSSRFGIRG |
| 562 | VDFFHVDYGRWERKHIRCASMSRVGLRG |
| 563 | GTFQHVDYGKWERKPIRCQSMSRVGYRR |
| 564 | VGYGKWERKLVRPASLS |
| 565 | VEKGETRRRLLNPTPHA |
| 566 | VGYGKWERKLIRSASTV |
| 567 | VEKPKPIRRLLNPGPNQ |
| 568 | VDYGKWERKLIRSASTS |
| 569 | VDYGKWERKTLRSRSLS |
| 570 | VGYGKWERRLVKSASSS |
| 571 | VDYGRWERKHIRCASMS |
| 572 | VERPKPIRRLLTPTPGC |
| 573 | PFRAPTIKILSKQFTEDDIDFWHVGYGKWERKLVRPAS |
| | LSGRRGLRR |
| 574 | PFRAPTVKILSKQFTDKDIDFSHVGYGKWERKMIRSA |
| | SISRLGL |
| 575 | DIAFRAPTVKILSKQFTDRDVDFSHVGYGKWERKLIR |
| | SASTVKYGL |
| 576 | DIRFKPPTINILSKDYTADCVDFWSVEKPKPIRRLLNPG |
| | PNQGPYPNTG |
| 577 | DIPFRAPTVKIHSKQFSHRDVDFSHVDYGKWERKTLRSR |
| | SLSRIGL |
| 578 | DIPFRAPTVKILAKQFSEKDIDFWHVGYGKWERRLVKSA |
| | SSSRFGI |
| 579 | DIPFRAPTVKILSKQFTDKDVDFFHVDYGRWERKHIRCA |
| | SMSRVGL |
| 580 | DIKYKPPTIKILSKDYTADCVDFWSVERPKPIRRLLTPTPGCG |
| 581 | ARTKQTARKSTGGKAPRKQLATKAARKSAPATGGVK |
| | KPHRYRPGTVA |
| 582 | SGRGKGGKGLGKGGAKRHRKVLRDNIQGITKPAI |

TABLE 2
Relative Fluorescence of Transfection of HeLa Cells

| SEQ ID NO | RF* | SEQ ID NO | RF |
|---|---|---|---|
| DOTMA | 7551 | 327 | 5345 |
| 107 | 75 | 328 | 32528 |
| 205 | 26368 | 332 | 9198 |
| 216 | 6181 | 335 | 26954 |
| 218 | 11179 | 336 | 25262 |
| 219 | 13721 | 338 | 6241 |
| 220 | 9398 | 341 | 4108 |
| 224 | 5559 | 342 | 33666 |
| 226 | 7340 | 343 | 5910 |
| 229 | 8816 | 344 | 14415 |
| 230 | 8944 | 345 | 8493 |
| 234 | 3867 | 347 | 22527 |
| 236 | 7202 | 348 | 22289 |
| 236 | 5042 | 349 | 4433 |
| 237 | 21288 | 350 | 13313 |
| 238 | 6067 | 351 | 12279 |
| 239 | 2717 | 352 | 32126 |
| 256 | 2243 | 353 | 29119 |
| 268 | 25885 | 354 | 4403 |
| 323 | 14067 | 355 | 32628 |
| 326 | 18946 | 357 | 22679 |
| | | 358 | 15132 |
| | | 359 | 3136 |
| | | 360 | 8516 |
| | | 361 | 3698 |
| | | 363 | 2590 |
| | | 365 | 36093 |
| | | 367 | 39008 |
| | | 369 | 14444 |
| | | 371 | 5180 |
| | | 373 | 8992 |
| | | 375 | 41076 |
| | | 377 | 15091 |
| | | 379 | 27424 |
| | | 381 | 22652 |
| | | 383 | 8012 |
| | | 450 | 21045 |
| | | 452 | 13888 |
| | | 454 | 34550 |
| | | 503 | 25368 |

*RF = Relative Fluorescence

TABLE 3
Relative Fluorescence of Transfection of HuVec Cells

| SEQ ID NO | RF* | SEQ ID NO | RF |
|---|---|---|---|
| DOTMA | 12230 | 328 | 3614 |
| 205 | 8309 | 332 | 4221 |
| 216 | 14693 | 335 | 6350 |
| 218 | 14827 | 336 | 5366 |
| 219 | 6237 | 338 | 12000 |
| 220 | 8737 | 341 | 15906 |
| 224 | 15945 | 342 | 12804 |
| 226 | 20864 | 343 | 16239 |
| 229 | 5452 | 344 | 11867 |
| 230 | 9067 | 345 | 2939 |
| 234 | 13956 | 347 | 20728 |
| 235 | 26940 | 348 | 21214 |
| 236 | 14418 | 349 | 22907 |
| 237 | 2302 | 350 | 13163 |
| 238 | 11083 | 351 | 15379 |
| 239 | 1404 | 352 | 3130 |
| 256 | 3030 | 353 | 8772 |
| 268 | 5403 | 354 | 19284 |
| 323 | 12815 | 355 | 14810 |
| 326 | 4932 | 357 | 6009 |
| 327 | 21018 | 358 | 25398 |
| | | 359 | 3024 |
| | | 360 | 24042 |
| | | 361 | 6017 |
| | | 363 | 1383 |
| | | 365 | 14764 |
| | | 367 | 13354 |
| | | 369 | 6536 |
| | | 371 | 10669 |
| | | 373 | 28811 |
| | | 375 | 5542 |
| | | 377 | 9326 |
| | | 379 | 14573 |
| | | 381 | 17542 |
| | | 383 | 20267 |
| | | 450 | 20911 |
| | | 452 | 1841 |
| | | 454 | 13336 |
| | | 503 | 12123 |

*RF = Relative Fluorescence

TABLE 4
Relative Fluorescence of Transfection of NL-1 iPS Cells

| SEQ ID NO | RF* | SEQ ID NO | RF |
|---|---|---|---|
| DOTMA | 8965 | 327 | 31383 |
| 107 | 1441 | 328 | 8611 |
| 205 | 13140 | 332 | 8725 |
| 216 | 19626 | 335 | 14424 |
| 218 | 17049 | 336 | 14875 |
| 219 | 5770 | 338 | 17288 |
| 220 | 15892 | 341 | 20446 |
| 224 | 25209 | 342 | 11493 |
| 226 | 24920 | 343 | 21726 |
| 229 | 13491 | 344 | 10895 |
| 230 | 14524 | 345 | 7836 |
| 234 | 14410 | 347 | 21865 |
| 236 | 43542 | 348 | 21645 |
| 236 | 22256 | 349 | 33103 |
| 237 | 8632 | 350 | 19132 |
| 238 | 15856 | 351 | 24646 |
| 239 | 4129 | 352 | 11628 |
| 256 | 1825 | 353 | 9248 |
| 268 | 15697 | 354 | 24043 |
| 323 | 21694 | 355 | 7910 |
| 326 | 9239 | 357 | 15624 |
| | | 358 | 21956 |
| | | 359 | 5548 |
| | | 360 | 27572 |
| | | 361 | 9363 |
| | | 363 | 4189 |
| | | 365 | 6364 |
| | | 367 | 17545 |
| | | 369 | 13124 |
| | | 371 | 17587 |
| | | 373 | 36290 |
| | | 375 | 5196 |
| | | 377 | 5474 |
| | | 379 | 6894 |
| | | 381 | 9644 |
| | | 383 | 21983 |
| | | 450 | 29856 |
| | | 452 | 10349 |
| | | 454 | 18100 |
| | | 503 | 19014 |

*RF = Relative Fluorescence

TABLE 5
Relative Fluorescence of Transfection of C2C12 Cells

| SEQ ID NO | RF* | SEQ ID NO | RF |
|---|---|---|---|
| DOTMA | 1289 | 328 | 5507 |
| 205 | 2102 | 332 | 537 |
| 216 | 1001 | 335 | 4689 |
| 218 | 3706 | 336 | 9647 |
| 219 | 1346 | 338 | 2290 |
| 220 | 1470 | 341 | 2141 |
| 224 | 1469 | 342 | 9496 |
| 226 | 1664 | 343 | 3218 |
| 229 | 1164 | 344 | 1678 |
| 230 | 2606 | 345 | 6569 |
| 234 | 681 | 347 | 4457 |
| 236 | 958 | 348 | 8625 |
| 236 | 2142 | 350 | 2631 |
| 237 | 6491 | 351 | 3530 |
| 238 | 1550 | 352 | 3324 |
| 239 | 10620 | 353 | 4291 |
| 256 | 3207 | 354 | 1912 |
| 268 | 2568 | 355 | 1900 |
| 323 | 7698 | 357 | 4506 |
| 326 | 8553 | 358 | 2271 |
| 327 | 2043 | 359 | 5693 |
| | | 360 | 2090 |
| | | 361 | 585 |
| | | 363 | 1080 |
| | | 365 | 5390 |
| | | 367 | 16207 |
| | | 369 | 3322 |
| | | 371 | 983 |
| | | 373 | 3037 |
| | | 375 | 5633 |
| | | 377 | 1710 |
| | | 379 | 3189 |
| | | 381 | 8834 |
| | | 383 | 1876 |
| | | 450 | 3795 |
| | | 452 | 21798 |
| | | 454 | 9280 |
| | | 501 | 5913 |
| | | 502 | 2589 |
| | | 503 | 3973 |

*RF = Relative Fluorescence

TABLE 6
Relative Fluorescence of Transfection of Human Fibroblast Cells

| SEQ ID NO | RF* | SEQ ID NO | RF |
|---|---|---|---|
| DOTMA | 3397 | 328 | 5641 |
| 205 | 8550 | 332 | 5178 |
| 216 | 3117 | 335 | 8534 |
| 218 | 11230 | 336 | 10449 |
| 219 | 7389 | 338 | 5625 |
| 220 | 4516 | 341 | 2284 |
| 224 | 5995 | 342 | 9525 |
| 226 | 3431 | 343 | 4590 |
| 229 | 6811 | 344 | 12704 |
| 230 | 11771 | 345 | 2816 |
| 234 | 4145 | 347 | 9255 |
| 236 | 3630 | 348 | 11802 |
| 236 | 3000 | 349 | 8810 |
| 237 | 2184 | 350 | 7105 |
| 238 | 3348 | 351 | 8061 |
| 239 | 4084 | 352 | 3059 |
| 256 | 2656 | 353 | 8653 |
| 268 | 4568 | 354 | 4998 |
| 323 | 4826 | 355 | 6821 |
| 326 | 3070 | 357 | 7167 |
| 327 | 4662 | 358 | 5665 |
| | | 359 | 3206 |
| | | 360 | 5347 |
| | | 361 | 7014 |
| | | 363 | 3008 |
| | | 365 | 5219 |
| | | 367 | 10036 |
| | | 369 | 7590 |
| | | 371 | 4584 |
| | | 373 | 4872 |
| | | 375 | 7305 |
| | | 377 | 6638 |
| | | 379 | 8520 |
| | | 381 | 9953 |
| | | 383 | 5896 |
| | | 450 | 6996 |
| | | 452 | 2779 |
| | | 454 | 7568 |
| | | 501 | 9473 |
| | | 503 | 13085 |

*RF = Relative Fluorescence

TABLE 7
Relative Fluorescence of Transfection of Jurkat Cells

| SEQ ID NO | RF* | SEQ ID NO | RF |
|---|---|---|---|
| DOTMA | 9084 | 328 | 4770 |
| 205 | 2298 | 332 | 3052 |
| 216 | 3570 | 335 | 4567 |
| 218 | 21451 | 336 | 4813 |
| 219 | 10762 | 338 | 4475 |
| 220 | 6428 | 341 | 18042 |
| 224 | 13170 | 342 | 12825 |
| 226 | 12976 | 343 | 23499 |
| 229 | 8079 | 344 | 10160 |
| 230 | 10229 | 345 | 2462 |
| 234 | 5441 | 347 | 17720 |
| 236 | 11006 | 348 | 15585 |
| 236 | 4593 | 349 | 23167 |
| 237 | 1501 | 350 | 9184 |
| 238 | 6278 | 351 | 9730 |
| 239 | 2343 | 352 | 3150 |
| 256 | 3322 | 353 | 11107 |
| 268 | 3236 | 354 | 16705 |
| 323 | 2900 | 355 | 8132 |
| 326 | 3018 | 357 | 2256 |
| 327 | 14546 | 358 | 18820 |
| | | 359 | 1880 |
| | | 360 | 16607 |
| | | 361 | 7006 |
| | | 363 | 2432 |
| | | 365 | 5298 |
| | | 367 | 4365 |
| | | 369 | 11085 |
| | | 371 | 6093 |
| | | 373 | 12628 |
| | | 375 | 4272 |
| | | 377 | 6785 |
| | | 379 | 7711 |
| | | 381 | 10806 |
| | | 383 | 14910 |
| | | 450 | 8779 |
| | | 452 | 2554 |
| | | 454 | 8224 |
| | | 501 | 12350 |
| | | 503 | 9411 |

*RF = Relative Fluorescence

TABLE 8
Visual Confirmation of Transfection of Cells

| Cell Line | SEQ ID NOs |
|---|---|
| Rat Cortical Neurons | 220, 236, 238, 323, 327, 336, 338, 341, |
| | 343, 347, 348, 350, 351, 352, 354, 367, |
| | 369, 373, 375, 377, 454 |
| THP-1 | 219, 229, 230, 239, 323, 328, 332, 341, |
| | 343, 350, 351, 357, 358, 375, 450, 454 |
| Human Skeletal Muscle | 218, 219, 230, 328, 336, 344, 350, 351, |
| | 353, 355, 365, 375 |

TABLE 9
Relative Fluorescence DOTMA/SEQ ID NO: 72/Peptide as Compared
with Lipofectamine 2000 for the Transfection of HeLa Cells

RF*

| | 0.1^{†} | 0.2^{†} | 0.3^{†} | 0.4^{†} |
|---|---|---|---|---|
| Transfection | | | | |
| Agent | | | | |
| Lipo^{#} | 12694 | 22154 | 21542 | 19286 |
| DOTMA/72^{§} | 10178 | 11133 | 8021 | 7032 |
| SEQ ID NO^{‡} | | | | |
| 218 | 12686 | 13103 | 10151 | 10081 |
| 219 | 12135 | 13841 | 13531 | 11712 |
| 236 | 5993 | 8846 | 8492 | 7316 |
| 237 | 8986 | 9753 | 8015 | 8288 |
| 268 | 10162 | 9934 | 10792 | 9519 |
| 323 | 15537 | 16620 | 10839 | 9244 |
| 326 | 6887 | 9487 | 10330 | 9227 |
| 328 | 11037 | 12229 | 10801 | 10951 |
| 335 | 12866 | 12458 | 11547 | 9753 |
| 336 | 14442 | 16702 | 13434 | 11334 |
| 341 | 6406 | 7469 | 8020 | 7546 |
| 342 | 41299 | 36040 | 28841 | 18351 |
| 343 | 6961 | 8626 | 8577 | 8160 |
| 344 | 13315 | 16387 | 12564 | 12511 |
| 347 | 23188 | 21493 | 16814 | 14140 |
| 348 | 17413 | 18615 | 16164 | 15054 |
| 350 | 24092 | 23238 | 19802 | 22255 |
| 351 | 7678 | 11499 | 10575 | 8791 |
| 353 | 36315 | 44772 | 36882 | 25802 |
| 355 | 18362 | 27990 | 22367 | 18289 |
| 357 | 7718 | 11798 | 9229 | 9107 |
| 358 | 12331 | 12683 | 13686 | 11097 |
| 360 | 9946 | 11168 | 10381 | 8995 |
| 365 | 26620 | 43775 | 36054 | 21540 |
| 367 | 10427 | 21411 | 23001 | 17529 |
| 369 | 8737 | 16706 | 14880 | 12645 |
| 375 | 22457 | 30891 | 34887 | 29024 |
| 377 | 39115 | 40072 | 32914 | 22457 |
| 379 | 50018 | 47049 | 39036 | 26448 |
| 381 | 34187 | 37463 | 27007 | 20088 |
| 450 | 11962 | 17346 | 12553 | 10241 |
| 452 | 6276 | 12671 | 12047 | 10918 |
| 454 | 34033 | 37099 | 27412 | 16023 |
| 501 | 23571 | 23993 | 20410 | 14749 |
| 503 | 12827 | 20006 | 17101 | 16206 |

*RF = Relative Fluorescence
^{†}Volume of DOTMA/SEQ ID NO: 72/Peptide in μL in well of a 96 well plate
^{#}Lipofectamine 2000
^{§}DOTMA and SEQ ID NO: 72 with no additional peptide
^{‡}Peptide mixed with DOTMA/SEQ ID NO: 72

### Example 2: Plant Movement Proteins

Cells were plated to so that on the day of transfection the cells were 70% confluent in 96 well tissue culture plates. A DOMTA/DOPE Lipid solution (1:1 molar ratio) at 2 mg/mL in water were mixed with an equal volume of plant virus movement protein derived peptides that had the nucleic acid binding moiety RRRRRRRRRRRR (SEQ ID NO:109) or KKKKKKKKKKKKKKKK (SEQ ID NO: 102) covalently linked to the N-terminus, or C-terminus of each peptide during peptide synthesis. Peptides were at 2 mg/mL in water. The DOTMA/DOPE and the plant virus movement peptide solution the was diluted 1 to 1 (v/v) in water. DOMTA/DOPE/PEPTIDE solutions were added to 0.1 mL of a plasmid DNA solution (EF1Alpha eGFP plasmid) at 10 μg/mL in OptiMEM.

All solutions were at room temperature. A volume of 0.2 mL of a 10 μg/mL solution of DNA was aliquoted into each well of a non-tissue cultured treated plate. 1-6 μL of transfection reagents were added to DNA solutions respectively. The transfection reagent and DNA solution was mixed by pipetting up and down twice. Transfection complexes were formed for 10 minutes. After 10 minutes, 0.01 or 0.02 mL of the transfection complex was added to cells. HuVEC, HeLa, human adult keratinocytes, human primary adult fibroblast, and A549 cells all respectively received 0.01 mL of the transfection complex. Human skeletal muscle cells, mouse C2C12 cells, and Jurkat, cells received 0.02 mL of the transfection complex. Cells were incubated for 42 hours at 37° C. at 5% CO₂. Plates were read on a fluorescent plate reader. Cells were also examined visually under a microscope to assess the extent of transfection (in terms of the percent of cells transfected) with a fluorescent microscope. Other modes of analysis, for example quantification with B-galactosidase or luciferase reports plasmids can also be used. If the plate reader did not show a sufficient signal to noise ratio, then plates were scored for cells transfected and those peptides that show increase over DOTMA alone were noted.

TABLE 10
Relative Fluorescence of Transfection of Hela Cells

| Transfection | | | | | | |
|---|---|---|---|---|---|---|
| Agent | | | | | | |
| SEQ ID NO | | | | | | |
| 543 | 12174 | 42502 | 38779 | 28451 | 13793 | 7705 |
| 545 | 11056 | 28529 | 28943 | 14693 | 13009 | 10902 |
| 544 | 10683 | 19151 | 19877 | 16908 | 12759 | 8938 |
| 540 | 6422 | 35653 | 27970 | 18620 | 13917 | 10336 |
| 541 | 17145 | 36476 | 37105 | 23644 | 20473 | 15523 |
| 542 | 9010 | 32901 | 47293 | 42152 | 35631 | 18856 |
| DOTMA | 4424 | 4782 | 5377 | 6992 | 9694 | 11720 |
| 1 μg/ml | | | | | | |
| 109 | 4157 | 5578 | 6606 | 8520 | 8007 | 7803 |
| 547 | 5899 | 9491 | 10275 | 11241 | 8829 | 7989 |
| 546 | 15190 | 15022 | 12772 | 11535 | 8117 | 7181 |
| Blank | 3912 | 3941 | 3860 | 3805 | 3990 | 3871 |

TABLE 11
Relative Fluorescence of Transfection
of Human Primary Fibroblast Cells

| | Transfection Agent | | | | | |
|---|---|---|---|---|---|---|
| | Rf | | | | | |
| SEQ ID NO | 1 μl | 1.5 μl | 2 μl | 3 μl | 4 μl | 6 μl |
| Blank | 26467 | 26415 | 26510 | 26317 | 26738 | 26620 |
| 543 | 30416 | 45401 | 39786 | 33091 | 28648 | 28698 |
| 545 | 34309 | 48184 | 37497 | 29067 | 28928 | 29256 |
| 544 | 31368 | 39311 | 37634 | 32892 | 31428 | 28367 |
| 540 | 29368 | 40610 | 35505 | 33853 | 33063 | 30219 |
| 541 | 35030 | 44894 | 42097 | 31837 | 29537 | 28921 |
| 542 | 30818 | 43609 | 46966 | 36343 | 33706 | 31787 |
| DOTMA | 28527 | 28021 | 28035 | 28646 | 29613 | 34911 |
| 1 μg/ml | | | | | | |
| 109 | 28368 | 30373 | 29858 | 29895 | 30941 | 29386 |
| 547 | 29141 | 33704 | 35175 | 35563 | 33951 | 30539 |
| 546 | 31980 | 39259 | 41747 | 35020 | 32220 | 29476 |
| Blank | 26822 | 26658 | 26624 | 25988 | 27112 | 26703 |

TABLE 12
Relative Fluorescence of Transfection of HuVEC Cells

| | Transfection Agent | | | | | |
|---|---|---|---|---|---|---|
| | Rf | | | | | |
| SEQ ID NO | 1 μl | 1.5 μl | 2 μl | 3 μl | 4 μl | 6 μl |
| Blank | 23420 | 23592 | 23275 | 23437 | 23969 | 23973 |
| 543 | 26622 | 36742 | 42802 | 40584 | 32754 | 25492 |
| 545 | 26545 | 36649 | 46890 | 34047 | 30893 | 27133 |
| 544 | 25788 | 28622 | 31245 | 34718 | 31159 | 28639 |
| 540 | 24771 | 34062 | 44744 | 44087 | 45137 | 30023 |
| 541 | 27405 | 46446 | OVER | 40401 | 31568 | 28125 |
| 542 | 24827 | 33896 | 42546 | 43231 | 35294 | 29326 |
| DOTMA | 24590 | 24571 | 24811 | 24463 | 26972 | 29850 |
| 1 μg/ml | | | | | | |
| 109 | 24603 | 25735 | 25421 | 26641 | 26529 | 25907 |
| 547 | 24554 | 26172 | 28255 | 32494 | 31180 | 26775 |
| 546 | 25990 | 32762 | 37899 | 35528 | 31614 | 27690 |
| Blank | 24151 | 23427 | 23055 | 23320 | 23689 | 24147 |

### Example 3: Formulation 1

Formulation 1 comprised a combination of cationic lipids, neutral lipids, and peptides, as described above. Specifically, the cationic lipids used in Formulation 1 included DPePE-Sp-OMe and DPB-Arg. These cationic lipids are compounds of Formula 1, where:

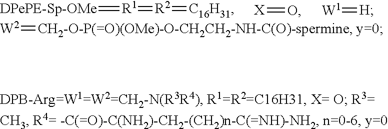

The neutral lipids included dioleoylphosphatidylethanolamine (DOPE), diphytanoylphosphatidylethanolamine (DPhPE), and 3-oleyloxy-2-hydroxy-1-acetamidopropane (HOAP).

The cationic lipid and the neutral lipid are combined in ethanol in the ratio of 1:8 (cationic lipid:neutral lipid). Formulation 1 was obtained by mixing the lipid with water and the peptide in the ratio of 0.2:1:1 (lipid mix:water:peptide mix). Specifically, the following peptides were used to prepare Formulation 1:
Peptide A: SEQ ID NO:507 covalently linked to SEQ ID NO:109 (4.1 mg/mL in HEPES);
Peptide B: SEQ ID NO:540 (4.1 mg/mL in HEPES);
Peptide C: SEQ ID NO:350 covalently linked to SEQ ID NO:109 (4.1 mg/mL in HEPES);
Peptide D: SEQ ID NO:47 (1 mg/mL in TRIS).

For Formulation 1, the lipid component comprised lipids in the molar ratio of 0.50:0.50:2.666:2.666:2.666 (DPePe-Sp-OMe:DPB-Arg:DOPE:DPhPE:HOAP). This translates to a cationic lipid:neutral lipid ratio of 1:8. The mixture of peptides in the formulation were present in the ratio of 1:2:2:0.5 by volume (Peptide A:Peptide B:Peptide C:Peptide D).

### Example 4: Transfection

Human Adult Dermal Fibroblast was obtained from ScienCell Research Laboratories (Carlsbad, CA) and grown according to manufacturer's instructions. Human Adult Keratinocytes were obtained from ThermoFisher Scientific and grown according to manufactures instructions in Keratinocyte-SFM. Cells were plated to obtain approximately 70% confluency the day of transfection in 96-well tissue culture treated plates. Green Fluorescent Protein expression plasmid was used in these experiments. Transfection complexes for Lipofectamine 3000 were prepared according to manufacturer's instructions. All solutions used to form transfection complexes were at room temperature for transfection. Into each well of a non-tissue culture treated plate containing 0.05 mL of OptIMEM were aliquoted 1.0, 1.5, 2.0, 3.0, 4.0 and 6.0 μL of the Formulation 1 transfection reagent. A volume of 0.05 mL of a 20 μg/mL solution of DNA in OptiMEM was aliquoted into each well of a non-tissue culture treated plate that contained transfection reagent aliquots. The transfection reagent and DNA solution were mixed by pipetting up and down twice. Transfection complexes were formed for 10 minutes at room temperature. After 10 minutes, 0.01 mL of the transfection complex was added to cells. Cells were incubated for 42 hours at 37° C. at 5% CO₂. Plates were read on a fluorescent plate reader. The results are shown in Tables 13 and 14. Cells were also examined visually under a microscope to assess the extent of transfection (regarding the percent of cells transfected) with a fluorescent microscope. Other modes of analysis, for example, quantification with B-galactosidase or luciferase reports plasmids, can also be used.

TABLE 13
Relative Fluorescence of Transfection
of Human Primary Adult Fibroblasts

| Volume of | | | |
|---|---|---|---|
| Transfection | | | |
| Reagent/well | Formulation 1 | Lipofectamine 3000 | Blank |
| 0.1 μL | 21851 | 9648 | 9373 |
| 0.15 μL | 21359 | 10041 | 9017 |
| 0.2 μL | 26793 | 12509 | 9135 |
| 0.3 μL | 35137 | 15193 | 9088 |
| 0.4 μL | 42224 | 16971 | 9200 |
| 0.6 μL | 42154 | 18095 | 9345 |

TABLE 14
Relative Fluorescence of Transfection
of Human Primary Adult Keratinocytes

| Volume of | | | |
|---|---|---|---|
| Transfection | | | |
| Reagent/well | Formulation 1 | Lipofectamine 3000 | Blank |
| 0.1 μL | 41003 | 15028 | 13843 |
| 0.15 μL | 36375 | 15981 | 13820 |
| 0.2 μL | 33300 | 15538 | 13674 |
| 0.3 μL | 23132 | 14780 | 13412 |
| 0.4 μL | 19578 | 15176 | 13987 |
| 0.6 μL | 17126 | 14934 | 13704 |

1. A transfection complex comprising a transfection enhancer and a cell surface ligand containing adhesion peptide, a plant virus movement protein, or peptide derived therefrom.

2. The transfection complex of claim 1, wherein the transfection enhancer is a peptide.

3. The transfection complex of claim 2, wherein the peptide is an amphipatic peptide.

4. The transfection complex of claim 2, wherein the peptide is a fusion peptide.

5. The transfection complex of claim 2, wherein the peptide is selected from the group consisting of SEQ ID NOs:1-580.

6. The transfection complex of claim 2, wherein the peptide is selected from the group consisting of SEQ ID NOs:107, 205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, and 503.

7. The transfection complex of claim 2, wherein the peptide is selected from the group consisting of SEQ ID NOs:205, 237, 268, 326, 328, 335, 336, 342, 347, 348, 352, 353, 355, 357, 358, 365, 367, 377, 379, 381, 450, 454, and 503.

8. The transfection complex of claim 2, wherein the peptide is selected from the group consisting of SEQ ID NOs:205, 216, 218, 219, 220, 224, 226, 229, 230, 234, 236, 236, 237, 238, 239, 256, 268, 323, 326, 327, 328, 332, 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, and 503.

9. The transfection complex of claim 2, wherein the peptide is selected from the group consisting of SEQ ID NOs:236, 358, and 373.

10. The transfection complex of claim 2, wherein the peptide is selected from the group consisting of SEQ ID NOs:107, 205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, and 503.

11. The transfection complex of claim 2, wherein the peptide is selected from the group consisting of SEQ ID NOs:216, 224, 226, 236, 236, 323, 327, 341, 343, 347, 348, 349, 350, 351, 354, 358, 360, 373, 383, 450, 454, and 503.

12. The transfection complex of claim 2, wherein the peptide is selected from the group consisting of SEQ ID NOs:205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 350, 351, 352, 353, 354, 355, 357, 358, 359, 360, 361, 363, 365, 367, 369, 371, 373, 375, 377, 379, 381, 383, 450, 452, 454, 501, 502, and 503.

13. The transfection complex of claim 2, wherein the peptide is selected from the group consisting of SEQ ID NOs:218, 230, 237, 239, 256, 323, 326, 328, 335, 336, 342, 343, 345, 347, 348, 352, 357, 359, 367, 375, 379, 381, 450, 452, 454, 501, 502, and 503.

14. The transfection complex of claim 2, wherein the peptide is selected from the group consisting of SEQ ID NOs:205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, 501, and 503.

15. The transfection complex of claim 2, wherein the peptide is selected from the group consisting of SEQ ID NOs:205, 218, 219, 229, 230, 335, 336, 342, 344, 348, 349, 350, 351, 353, 355, 357, 361, 367, 369, 375, 379, 381, 450, 454, 501, and 503.

16. The transfection complex of claim 2, wherein the peptide is selected from the group consisting of SEQ ID NOs:205, 216, 218, 219, 220, 224, 226, 229, 230, 234, 236, 236, 237, 238, 239, 256, 268, 323, 326, 327, 328, 332, 335, 336, 338, 341, 342, 343, 344, 345, 347, 348, 349, 350, 351, 352, 353, 354, 355, 357, 358, 359, 360, 361, 363, 365, 367, 369, 371, 373, 375, 377, 379, 381, 383, 450, 452, 454, 501, and 503.

17. The transfection complex of claim 2, wherein the peptide is selected from the group consisting of SEQ ID NOs:218, 349, and 358.

18. The transfection complex of claim 2, wherein the peptide is selected from the group consisting of SEQ ID NOs: 220, 236, 238, 323, 327, 336, 338, 341, 343, 347, 348, 350, 351, 352, 354, 367, 369, 373, 375, 377, and 454.

19. The transfection complex of claim 1, wherein the peptide is selected from the group consisting of SEQ ID NOs:219, 229, 230, 239, 323, 328, 332, 341, 343, 350, 351, 357, 358, 375, 450, and 454.

20. The transfection complex of claim 2, wherein the peptide is selected from the group consisting of SEQ ID NOs: 218, 219, 230, 328, 336, 344, 350, 351, 353, 355, 365, and 375.

21-77. (canceled)
